(19) United States
(12) Patent Application Publication
(10) Pub. No.: US 2006/0004423 A1
(43) Pub. Date: Jan. 5, 2006

(54) Methods and systems to provide therapy or alleviate symptoms of chronic headache, transformed migraine, and occipital neuralgia by providing rectangular and/or complex electrical pulses to occipital nerves

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/223,077

(22) Filed: Sep. 9, 2005

Related U.S. Application Data
(63) Continuation-in-part of application No. 10/841,995, filed on May 8, 2004.
Application No. 10/841,995 is a continuation-in-part of application No. 10/196,533, filed on Jul. 16, 2002.
Application No. 10/841,995 is a continuation-in-part of application No. 10/142,298, filed on May 9, 2002.

(51) Int. Cl. A61N 1/18

(52) U.S. Cl. 607/46; 607/45

(57) ABSTRACT

A method and system for providing rectangular and/or complex electrical pulses to occipital nerves, to provide therapy for at least one of chronic headache, transformed migraine, and occipital neuralgia comprises implantable and external components. Complex electrical pulses comprises pulses which are configured to be one of non-rectangular, multi-level, biphasic, or pulses with varying amplitude during the pulse. The electrical pulses to occipital nerves may be stimulating and/or blocking. The stimulation and/or blocking to occipital nerves may be provided using one of the following pulse generation means: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with a magnet; d) a programmable implantable pulse generator (IPG); e) a combination implantable device comprising both a stimulus-receiver and a programmable implantable pulse generator (IPG); and f) an implantable pulse generator (IPG) comprising a rechargeable battery. The pulse generator means comprises predetermined/pre-packaged programs. In one embodiment, the pulse generation means may also comprise telemetry means, for remote interrogation and/or programming of said pulse generation means, utilizing a wide area network.

This application is a continuation of application Ser. No. 10/841,995 filed May 8, 2004, entitled “METHOD AND SYSTEM FOR MODULATING THE VAGUS NERVE (10^{TH} CRANIAL NERVE) WITH ELECTRICAL PULSES USING IMPLANTED AND EXTERNAL COMPONANTS, TO PROVIDE THERAPY FOR NEUROLOGICAL AND NEUROPSYCHIATRIC DISORDERS”, which is a continuation of application Ser. No 10/196,533 filed Jul. 16, 2002, which is a continuation of application Ser. No. 10/142,298 filed on May 9, 2002. The prior applications being incorporated herein in their entirety by reference, and priority is claimed from the above applications.

### FIELD OF INVENTION

The present invention relates to neuromodulation, more specifically to provide therapy or alleviate symptoms of chronic headache, transformed migraine, or occipital neuralgia by selectively stimulating/modulating occipital nerves by providing rectangular and/or complex electrical pulses to occipital nerves.

### BACKGROUND

Clinical medical research has shown that occipital nerve(s) stimulation provides excellent benefits for chronic headaches, transformed migraine, and occipital neuralgia. Transformed Migraine (TM) and occipital neuralgia (ON) are distinct, clinically diverse, cervicocranial syndromes involving the posterior occiput. Both often manifest with life-altering disabling pain refractory to conventional therapy.

Transformed Migraine (TM) is a nonparoxysmal cervical tension and secondary radiating posterior headache pain syndrome occurring daily or almost daily, the etiology of which is unknown. Patients have a prior history of International Headache Society classification (HIS) episodic migraine with increasing headache frequency, and decreasing severity of migrainous features. Most experience episodic symptoms, including aura (15%), and respond to pharmacologic management. A significant number (up to 6%) of 38,000,000 migraine sufferers or 2,200,000 however, develop in the setting of symptomatic medication overuse and/or are refractory to conservative pharmacologic treatment. Recent theory suggests that this disabling TM “neuropathic subset” may be refractory due to the involvement of the trigeminocervical complex. Clinical investigators have also described a clinical correlation between subcutaneous, cylindrical C1-2-3 (PNS) and the reduction of (TM) central sensitization and disability.

Occipital neuralgia (ON) is characterized by paroxyms of pain occurring within the distribution of the greater and/or lesser occipital nerves. FIG. 1 shows the distribution of occipital nerves including greater occipital nerve 21, lesser occipital nerve 23, and the third occipital nerve 25. The pain of occipital neuralgia may radiate anteriorly to the ipsilateral frontal or retro-orbital regions of the head. Extreme localized tenderness if often encountered upon palpation over the occipital notches with reproduction of focal and radiating pain. Though known causes include closed head injury, direct occipital nerve trauma, neuroma formation, or upper cervical root compression (spondylosis or ligamentous hypertrophy), most patients have no demonstrable lesion.

Treatment options for intractable occipital nerve pain refractory to medication usually involves chemical, thermal, or surgical ablation procedures following diagnostic local anesthetic blockade. Surgical approaches include neurolysis or nerve sectioning of either the peripheral nerve in the occipital scalp or at the upper cervical dorsal root exit zone (extradural). Foraminal decompression of C2 roots as well as C2 ganglionectomy have also been effective in selected cases.

Persistent occipital neuralgia (ON) can produce severe headaches that may not be controllable by conservative or surgical approaches. In such cases implantable electrical stimulation is a viable alternative. The pain relief methodology of this invention is related to, and is supported by the widely known “gate control theory” of pain, which is summarized below.

Most nerves in the human body are composed of thousands of fibers of different sizes. This is shown schematically in FIG. 2. In a cross section of peripheral nerve it is seen that the diameter of individual fibers vary substantially, as is shown schematically in FIG. 3. The largest nerve fibers are approximately 20 μm in diameter and are heavily myelinated (i.e., have a myelin sheath, constituting a substance largely composed of fat), whereas the smallest nerve fibers are less than 1 μm in diameter and are unmyelinated.

The diameters of group A and group B fibers include the thickness of the myelin sheaths. Group A is further subdivided into alpha, beta, gamma, and delta fibers in decreasing order of size: There is some overlapping of the diameters of the A, B, and C groups because physiological properties, especially in the form of the action potential, are taken into consideration when defining the groups. The smallest fibers (group C) are unmyelinated and have the slowest conduction rate, whereas the myelinated fibers of group B and group A exhibit rates of conduction that progressively increase with diameter.

In the body, natural neural mechanisms exist to modulate pain transmission and perception. Shown in conjunction with FIG. 4, the gate control theory of pain suggests that:

1) A pain “gate” exists in the dorsal horn (substantia gelatinosa) where impulses from small unmyelinated pain fibers and large touch (A beta) fibers enter the cord.

2) If impulses along the pain fibers outnumber those transmitted along the touch fibers, the gate opens and pain impulses are transmitted. If the reverse is true, the gate is closed by enkephalin-releasing interneurons in the spinal cord that inhibit transmission of both touch and pain impulses, thus reducing pain perception.

When type A delta and type C pain fibers transmit through to their transmission neurons in the spinothalmic pathway, pain impulses are transmitted to the cerebral cortex. Descending control of pain transmission (analgesia) is mediated by descending central fibers that synapse with small enkephalin-releasing interneurons in the dorsal horn that make inhibitory synapses with the afferent pain fibers. Activation of these interneurons inhibits pain transmission by preventing their release of substance P.

It has been found that (1) threshold stimulation of the large touch fibers results in a burst of firing in the substantia gelatinosa cells, followed by a brief period of inhibited pain transmission (it does close the pain “gate”), and (2) it has been amply proven that direct stimulation, or even transcutaneous electrical nerve stimulation (TENS), of dorsal column (large-diameter touch) fibers does provide extended pain relief.

It has been known that our natural opiates (beta endorphins and enkephalins) are released in the brain when we are in pain and act to reduce its perception. Hypnosis, natural childbirth techniques, morphine, and stimulus-induced analgesia all tap into these natural-opiate pathways, which originate in certain brain regions. These regions, which include the periventricular gray matter of the hypothalamus and the periaqueductal gray matter of the midbrain, oversee descending pain suppressor fibers that synapse in the dorsal horns. When transmitting, these fibers (most importantly some from the medullary raphe magnus) produce analgesia, presumably by synapsing with opiate (enkephalin) releasing interneurons that in turn actively inhibit forward transmission of pain inputs (FIG. 4). The mechanism of this inhibition appears to be that enkephalin blocks Ca²⁺ influx into the sensory terminals, thereby blocking their release of substance P. However, this is only one mechanism of pain modulation. A variety of other neurotransmitter receptor systems in the dorsal horn also regulate pain perception.

In the methods and systems of this invention, electrical pulses are provided to occipital nerve(s), utilizing implantable and external components. Rectangular and/or complex electrical pulses may be provided utilizing predetermined/pre-packaged programs. Complex electrical pulses comprise at least one of multi-level pulses, biphasic pulses, non-rectangular pulses, or pulses with varying amplitude during the pulse. Predetermined/pre-packaged programs of therapy define the variable parameters comprising, pulse amplitude, pulse width, pulse frequency, electrode pair selection, and on-time and off-time sequence.

### PRIOR ART

U.S. Pat. No. 6,505,075 B1 (Weiner, R. L.) and U.S. patent application Ser. No. 0198572 A1 (Weiner, R. L.) are generally directed to method and apparatus for peripheral nerve stimulation including treating intractable occipital neuralgia using percutaneous peripheral nerve electrostimulation. Even though electrical stimulation is utilized, it is not clear from the disclosure what type of electrical pulses are used.

U.S. Pat. No. 6,735,475 B1 (Whitehurst et al.) is generally directed to the use of BIONS for providing stimulation therapy for headache and/or facial pain. Because of its size, the BION(s)® may be implanted via minimal surgical procedure.

U.S. patent application Ser. No. 0154419 A1 (Whitehurst et al.) is generally directed to stimulating nerve originating in an upper cervical spine area of the patient, utilizing one or more microstimulators or BION(s)®.

U.S. patent application Ser. No. 0102006 A1 (Whitehurst et al.) is generally directed to treating headaches and neuralgia using an inductively coupled system.

U.S. patent application Ser. No. 0143789 A1 (Whitehurst et al.) is generally directed to stimulating a peripheral nerve to treat chronic pain using an inductively coupled system such as a BION(s)®.

### SUMMARY OF THE INVENTION

The methods and systems of the current invention provides neuromodulation therapy for at least one of chronic headache, transformed migraine, and occipital neuralgia by providing rectangular or complex electrical pulses to occipital nerves or branches, for selective stimulation and/or blocking. The method and system comprises both implantable and external components. The power source may also be external or implanted in the body.

Accordingly, it is one object of the invention to provide predetermined rectangular and/or complex electrical pulses to occipital nerves or branches, for stimulation and/or blocking, to provide therapy or to alleviate symptoms for at least one of chronic headache, transformed migraine, and occipital neuralgia.

It is another object of the invention to provide predetermined/pre-packaged programs for delivering therapy. Predetermined/pre-packaged programs of therapy define the variable parameters comprising, pulse amplitude, pulse width, pulse frequency, electrode pair selection, and on-time and off-time sequence.

In one aspect of the invention, the electrical pulses are provided using an implanted stimulus-receiver adopted to work in conjunction with an external stimulator.

In another aspect of the invention, the electrical pulses are provided using an implanted stimulus-receiver which comprises a high value capacitor for storing charge, and is adapted to work in conjunction with an external stimulator.

In another aspect of the invention, the electrical pulses are provided using a programmer-less implantable pulse generator (IPG) which can be programmed with a magnet.

In another aspect of the invention, the electrical pulses are provided using a programmable implantable pulse generator (IPG).

In another aspect of the invention, the electrical pulses are provided using a combination device which comprises both a stimulus-receiver and a programmable implantable pulse generator.

In another aspect of the invention, the electrical pulses are provided using an implantable pulse generator which comprises a re-chargeable battery.

In another aspect of the invention, pulsed electrical stimulation and/or blocking pulses may be provided.

In another aspect of the invention, the nerve blocking comprises at least one from a group consisting of: DC or anodal block, Wedenski block, and Collision block.

In another aspect of the invention, the external components such as the external stimulator or programmer comprise telemetry means adapted to be networked, for remote interrogation or remote programming of the device.

In yet another aspect of the invention, the implanted lead comprises at least one electrode selected from the group comprising button electrodes, or cylindrical electrodes.

Various other features, objects and advantages of the invention will be made apparent from the following description taken together with the drawings.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a diagram depicting anatomy of the occipital nerves.

FIG. 2 is a diagram of the structure of a nerve.

FIG. 3 is a diagram showing different types of nerve fibers.

FIG. 4 is a diagram depicting a cross section of spinal cord afferent primary nociceptive fibers.

FIGS. 5A and 5B depict placement of lead pair relative to occipital nerves in a patient.

FIGS. 5C and 5D depict placement of a single lead relative to occipital nerves in a patient.

FIG. 6 is a simplified block diagram depicting supplying amplitude and pulse width modulated electromagnetic pulses to an implanted coil.

FIG. 7 is a diagram depicting placement of an external stimulator relative to an implanted stimulus receiver.

FIG. 8 is a diagram depicting placement of external (primary) coil relative to an implanted (secondary) coil.

FIGS. 9A and 9B depict another embodiment showing placement of external stimulator relative to an implanted secondary coil.

FIG. 10 is a schematic of the passive circuitry in the implanted stimulus-receiver.

FIG. 11A is a schematic of an alternative embodiment of the implanted stimulus-receiver.

FIG. 11B is another alternative embodiment of the implanted stimulus-receiver.

FIGS. 12A and 12B show coupling of primary coil of the external stimulator and secondary coil of the implanted stimulus-receiver.

FIG. 13 is a top-level block diagram of the external stimulator and proximity sensing mechanism.

FIG. 14 is a diagram showing the proximity sensor circuitry.

FIG. 15A shows the pulse train to be transmitted to the occipital nerves.

FIG. 15B shows the ramp-up and ramp-down characteristic of the pulse train.

FIG. 16 is a schematic diagram showing a pair of paddle leads.

FIG. 17A is a schematic diagram showing a pair of cylindrical leads.

FIG. 17B is a schematic diagram showing both distal and terminal end of a lead.

FIG. 18 is a schematic diagram showing a single paddle lead.

FIG. 19 is a schematic diagram showing a single cylindrical lead.

FIG. 20 is a schematic diagram showing the implantable lead and one form of stimulus-receiver.

FIG. 21 is a block diagram showing schematically the functioning of the external transmitter and the implanted lead stimulus-receiver.

FIG. 22 is a schematic block diagram showing a system for neuromodulation of occipital nerves, with an implanted component which is both RF coupled and comprises a high-value capacitor power source.

FIG. 23 is a simplified block diagram showing control of the implantable neurostimulator with a magnet.

FIG. 24 is a schematic diagram showing implementation of a multi-state converter.

FIG. 25 is a schematic diagram depicting digital circuitry for state machine.

FIG. 26 is a simplified block diagram of an implantable pulse generator.

FIG. 27 is a functional block diagram of a microprocessor-based implantable pulse generator.

FIG. 28 shows details of implanted pulse generator.

FIGS. 29A and 29B shows details of digital components of the implantable circuitry.

FIG. 30A shows a schematic diagram of the register file, timers and ROM/RAM.

FIG. 30B shows datapath and control of custom-designed microprocessor based pulse generator.

FIG. 31 is a block diagram for generation of a pre-determined stimulation pulse.

FIG. 32 is a simplified schematic for delivering stimulation pulses.

FIG. 33 is a circuit diagram of a voltage doubler.

FIG. 34A is a diagram depicting ramping-up of a pulse train.

FIG. 34B depicts rectangular pulses.

FIGS. 34C, 34D, and 34E depict multi-step pulses.

FIGS. 34F, 34G, and 34H depict complex pulse trains.

FIGS. 34-I and 34J depict step pulses used in conjunction with tripolar electrodes.

FIGS. 34K and 34L depict biphasic pulses which can be used in conjunction with tripolar electrodes.

FIGS. 34M and 34N depict modified square pulses which can be used in conjunction with tripolar electrodes.

FIGS. 35A and 35B are diagrams showing communication of programmer with the implanted stimulator.

FIGS. 36A and 36B show diagrammatically encoding and decoding of programming pulses.

FIG. 37 is a simplified overall block diagram of implanted pulse generator (IPG) programmer.

FIG. 38 shows a programmer head positioning circuit.

FIG. 39 depicts typical encoding and modulation of programming messages.

FIG. 40 shows decoding one bit of the signal from FIG. 39.

FIG. 41 shows a diagram of receiving and decoding circuitry for programming data.

FIG. 42 shows a diagram of receiving and decoding circuitry for telemetry data.

FIG. 43 is a block diagram of a battery status test circuit.

FIG. 44 is a diagram showing the two modules of the implanted pulse generator (IPG).

FIG. 45A depicts coil around the titanium case with two feedthroughs for a bipolar configuration.

FIG. 45B depicts coil around the titanium case with one feedthrough for a unipolar configuration.

FIG. 45C depicts two feedthroughs for the external coil which are common with the feedthroughs for the lead terminal.

FIG. 45D depicts one feedthrough for the external coil which is common to the feedthrough for the lead terminal.

FIG. 46 shows a block diagram of an implantable stimulator which can be used as a stimulus-receiver or an implanted pulse generator with rechargeable battery.

FIG. 47 is a block diagram highlighting battery charging circuit of the implantable stimulator of FIG. 46.

FIG. 48 is a schematic diagram highlighting stimulus-receiver portion of implanted stimulator of one embodiment.

FIG. 49A depicts bipolar version of stimulus-receiver module.

FIG. 49B depicts unipolar version of stimulus-receiver module.

FIG. 50 depicts power source select circuit.

FIG. 51A shows energy density of different types of batteries.

FIG. 51B shows discharge curves for different types of batteries.

FIG. 52 depicts externalizing recharge and telemetry coil from the titanium case.

FIGS. 53A and 53B depict recharge coil on the titanium case with a magnetic shield in-between.

FIG. 54 shows in block diagram form an implantable rechargeable pulse generator.

FIG. 55 depicts in block diagram form the implanted and external components of an implanted rechargeable system.

FIG. 56 depicts the alignment function of rechargeable implantable pulse generator.

FIG. 57 is a block diagram of the external recharger.

FIG. 58 depicts remote monitoring of stimulation devices.

FIG. 59 is an overall schematic diagram of the external stimulator, showing wireless communication.

FIG. 60 is a schematic diagram showing application of Wireless Application Protocol (WAP).

FIG. 61 is a simplified block diagram of the networking interface board.

FIGS. 62A and 62B are simplified diagrams showing communication of modified PDA/phone with an external stimulator via a cellular tower/base station.

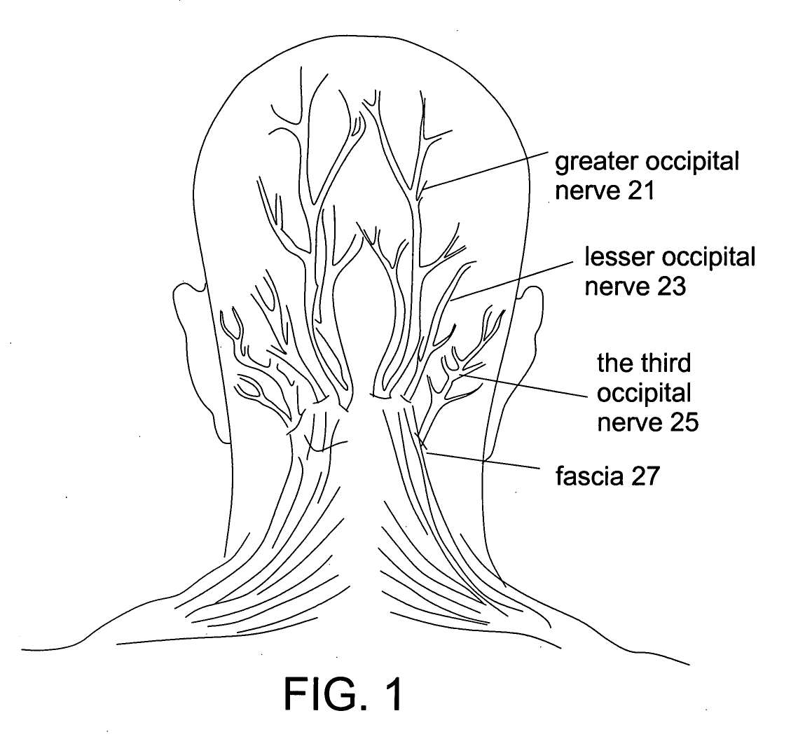

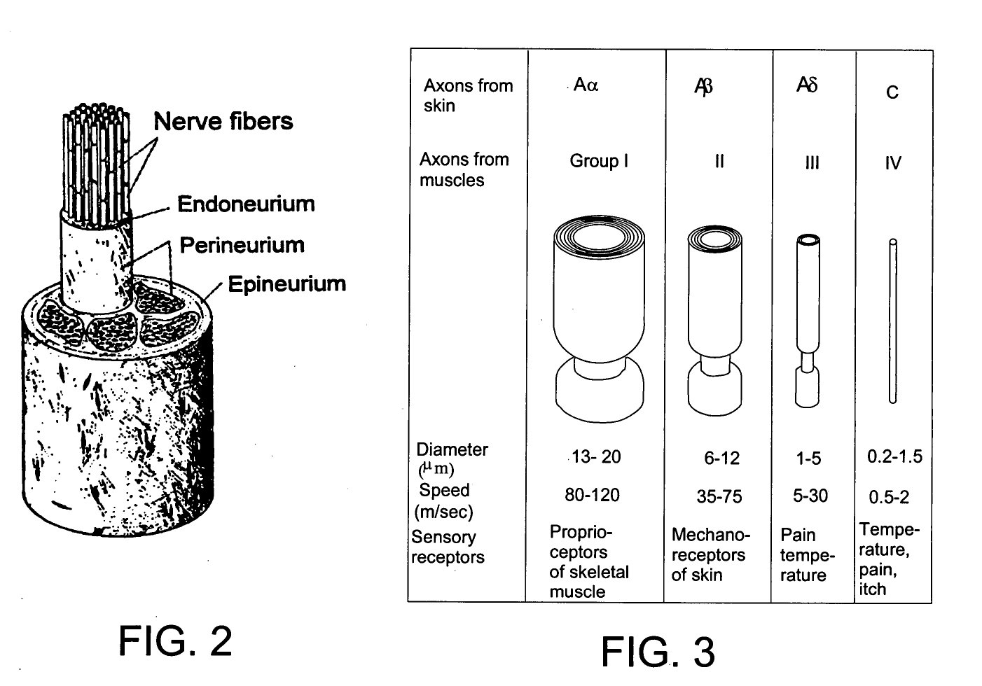

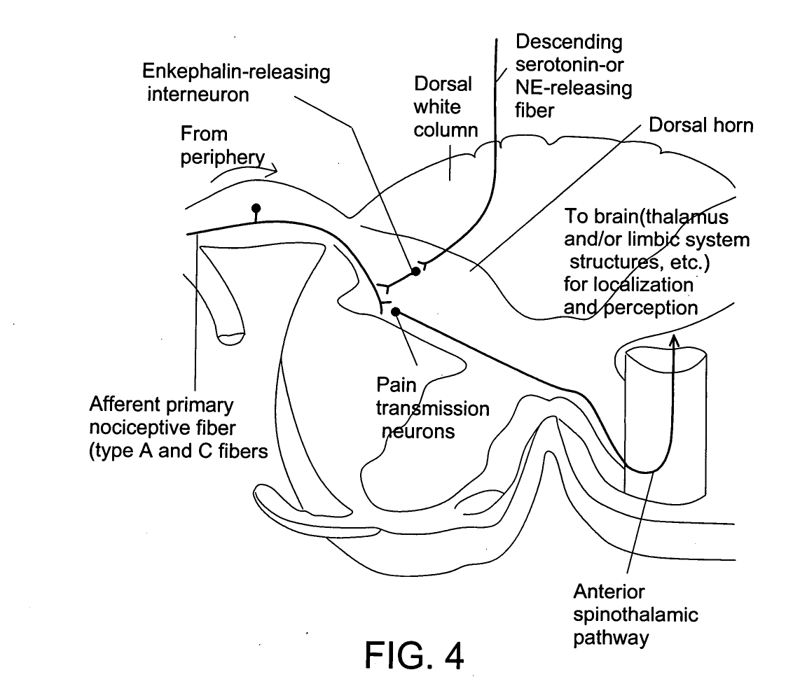

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

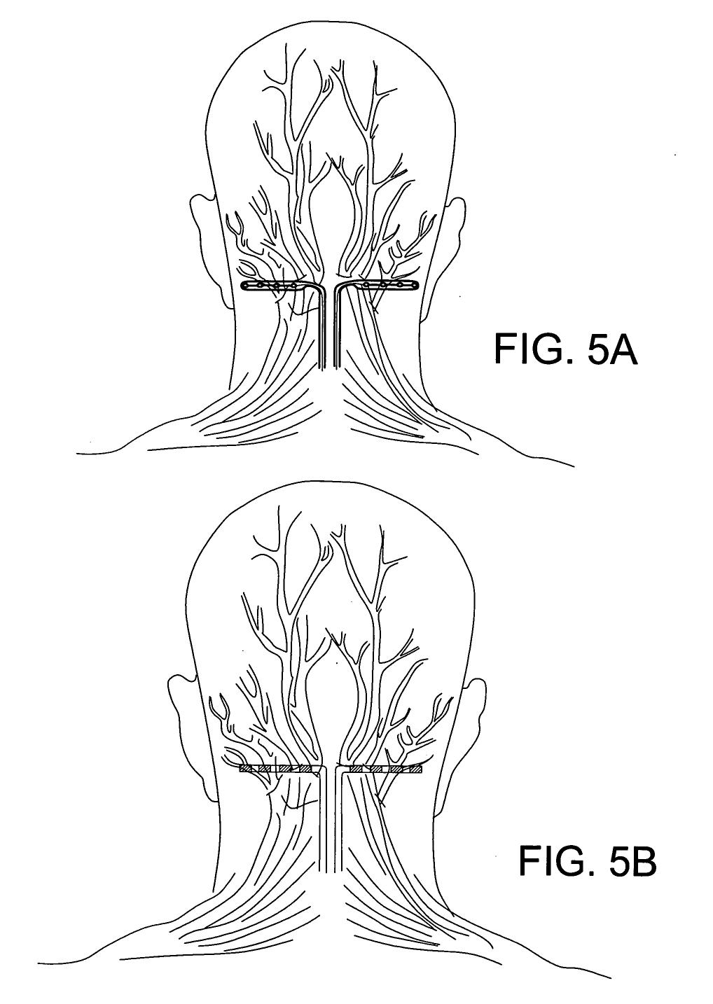

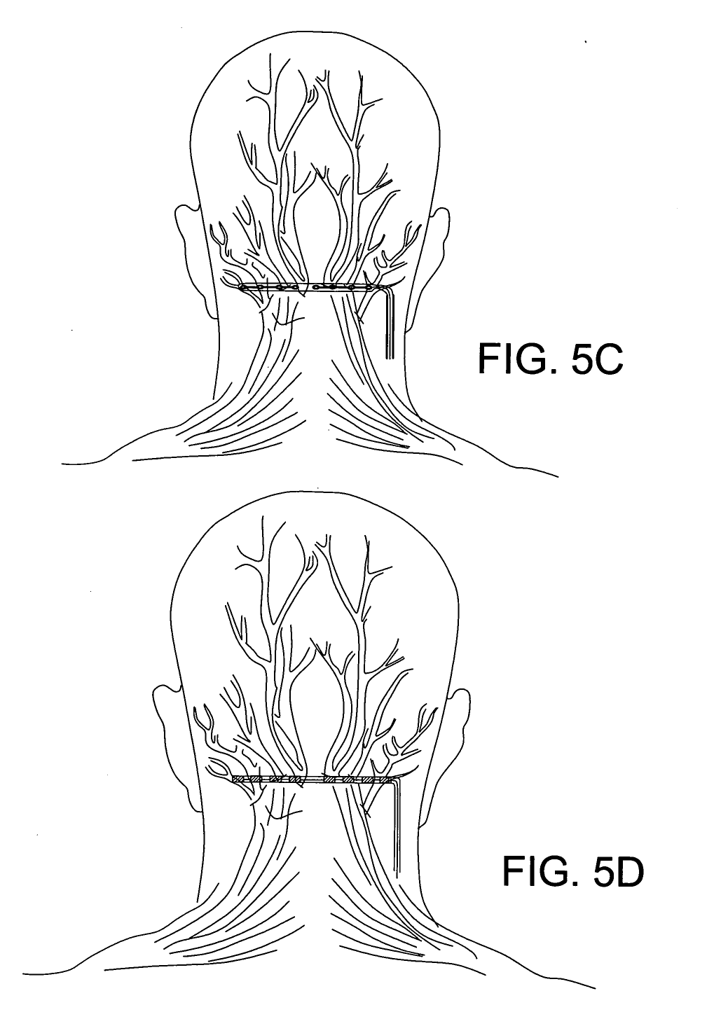

In the methods and systems of this invention, selective pulsed electrical stimulation is applied to occipital nerves to provide therapy or alleviate symptoms for at least one of chronic headache, transformed migraine, and occipital neuralgia. One or two leads are surgically implanted in the fascia in close proximity to the occipital nerves, as is shown in conjunction with FIGS. 5A, 5B, 5C, and 5D. A midline or lateral incision may be used. The lead or leads are placed in the facia with the electrodes at the appropriate level (approximately around the C1-2-3 level). The terminal (proximal) end of the lead is tunneled subcutaneously. A pulse generator means is connected to the terminal (proximal) end of the lead. The power source may be external, implantable, or a combination device.

Many of the patients may end up with more than one type of pulse generator in their lifetime. In the methodology of this invention, an implanted lead(s) has a terminal end which is compatible with different embodiments of pulse generators disclosed in this application. Once the lead is implanted in a patient, any embodiment of the pulse generator disclosed in this application, may be implanted in the patient. Furthermore, at replacement the same embodiment or a different embodiment may be implanted in the patient using the same lead(s). This may be repeated as long as the implanted lead(s) is/are functional and maintain its integrity.

As one example, without limitation, an implanted stimulus-receiver in conjunction with an external stimulator may be used initially. At a later time, the pulse generator may be exchanged for a more elaborate implanted pulse generator (IPG) model, keeping the same lead. Some examples of stimulation and power sources that may be used for the practice of this invention, and disclosed in this application, comprise:

a) an implanted stimulus-receiver used with an external stimulator;

b) an implanted stimulus-receiver comprising a high value capacitor for storing charge, used in conjunction with an external stimulator;

c) a programmer-less implantable pulse generator (IPG) which is operable with a magnet;

d) a programmable implantable pulse generator;

e) a combination implantable device comprising both a stimulus-receiver and a programmable IPG; and

f) an IPG comprising a rechargeable battery.

All of these pulse generator means can generate and emit rectangular and complex electrical pulses. Complex electrical pulses comprise at least one of multi-level pulses, biphasic pulses, non-rectangular pulses, or pulses with varying amplitude during the pulse.

### Implanted Stimulus-Receiver With An External Stimulator

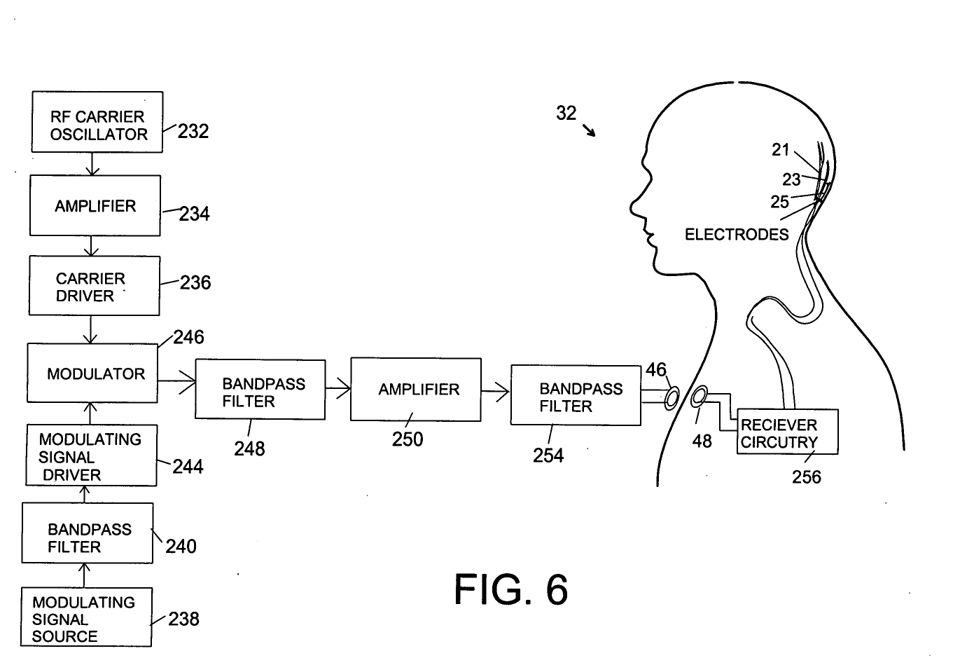

The selective stimulation of various nerve fibers of occipital nerves, as performed by one embodiment of the method and system of this invention is shown schematically in FIG. 6, as a block diagram. A modulator 246 receives analog (sine wave) high frequency “carrier” signal and modulating signal. The modulating signal can be multilevel digital, binary, or even an analog signal. In this embodiment, mostly multilevel digital type modulating signals are used. The modulated signal is amplified 250, conditioned 254, and transmitted via a primary coil 46 which is external to the body. A secondary coil 48 of an implanted stimulus receiver, receives, demodulates, and delivers these pulses to the occipital nerves via an electrode pair such as electrodes 61 and 62 (or a different electrode pair). The receiver circuitry 256 is described later.

The carrier frequency is optimized. One preferred embodiment utilizes electrical signals of around 1 Mega-Hertz, even though other frequencies can be used. Low frequencies are generally not suitable because of energy requirements for longer wavelengths, whereas higher frequencies are absorbed by the tissues and are converted to heat, which again results in power losses.

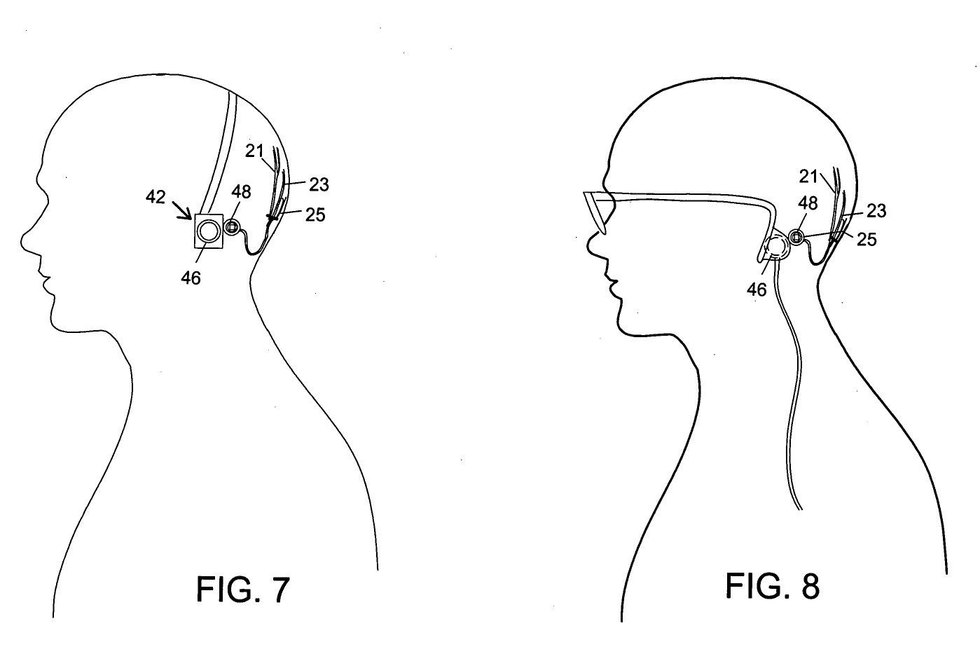

Shown in conjunction with FIGS. 7 and 8, the primary (external) coil 46 is in close position to the secondary (implanted) coil 48. Shown in conjunction with FIG. 7, the external stimulation package may be attached to a head-band for convenience. Alternatively, shown in conjunction with FIG. 8, the primary coil 46 may be positioned with the aid of eye-glasses, and the stimulator electronics package may be placed in a pocket or clipped to a belt for example.

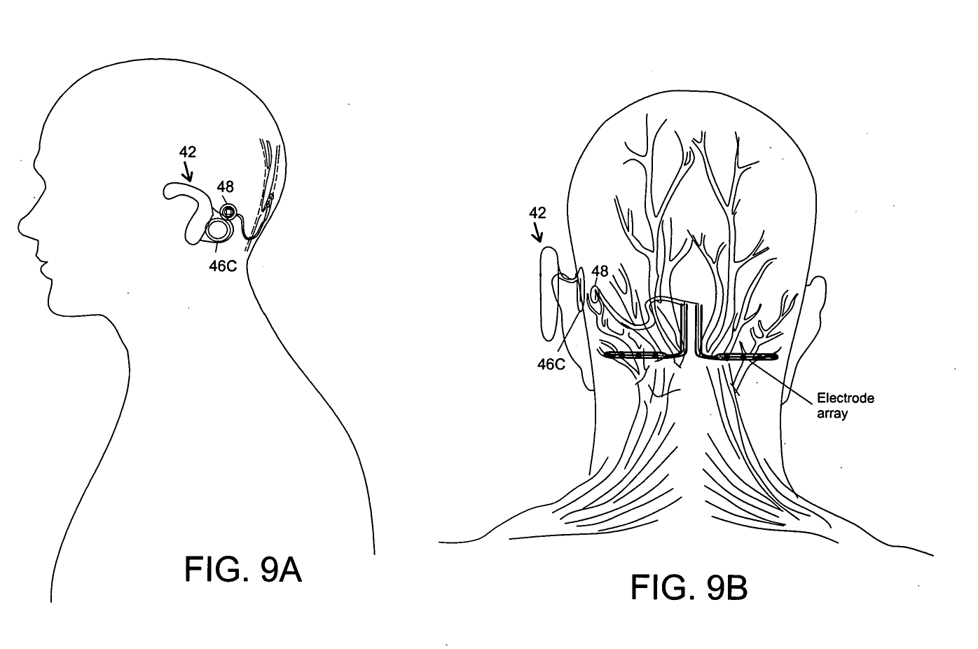

In one embodiment, as shown in conjunction with FIGS. 9A and 9B, the external stimulator 42 is anchored to the ear, and the implanted stimulus-receiver package is implanted subcutaneouly behind the ear. The primary (external) coil 46 of the external stimulator 42 is inductively coupled to the secondary (implanted) coil 48 of the implanted stimulus-receiver 34. The implantable stimulus-receiver 34 has circuitry at the proximal end, and has eight stimulating electrodes at the distal end. The electrode array may also comprise more than eight, or less than eight electrodes.

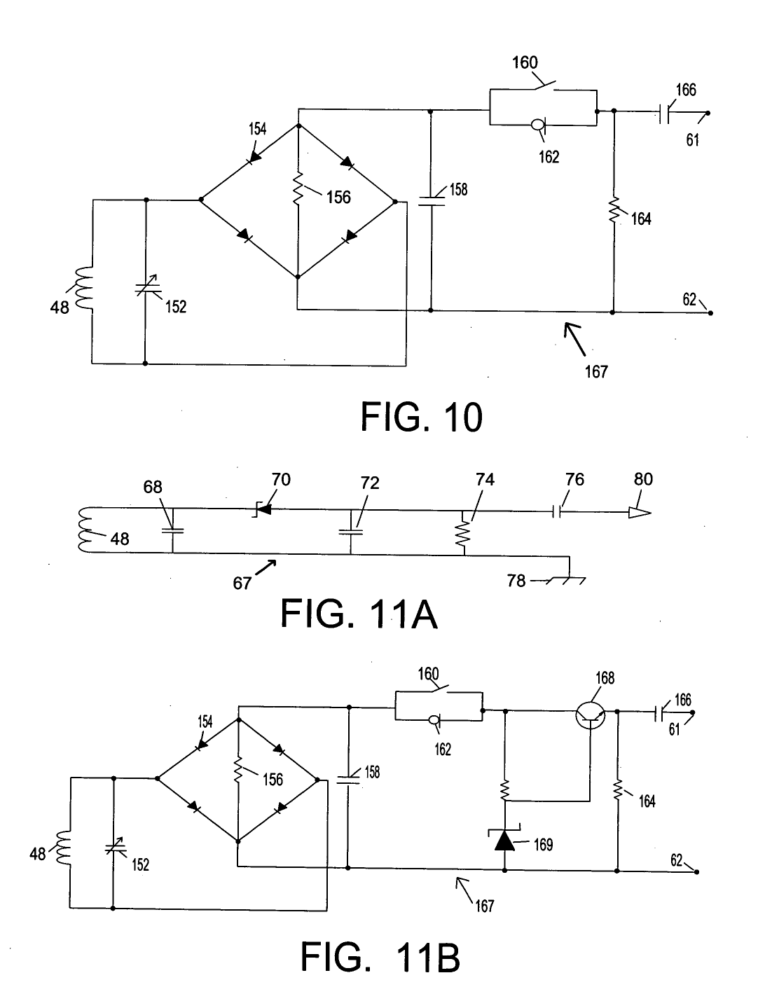

The circuitry contained in the proximal end of the implantable stimulus-receiver 34 is shown schematically in FIG. 10, for one embodiment. In this embodiment, the circuit uses all passive components. Approximately 25 turn copper wire of 30 gauge, or comparable thickness, is used for the primary coil 46 and secondary coil 48. This wire is concentrically wound with the windings all in one plane. The frequency of the pulse-waveform delivered to the implanted coil 48 can vary, and so a variable capacitor 152 provides ability to tune secondary implanted circuit 167 to the signal from the primary coil 46. The pulse signal from secondary (implanted) coil 48 is rectified by the diode bridge 154 and frequency reduction obtained by capacitor 158 and resistor 164. The last component in line is capacitor 166, used for isolating the output signal from the electrode wire. The return path of signal from cathode 61 will be through anode 62 placed in proximity to the cathode 61 for “Bipolar” stimulation. In this embodiment bipolar mode of stimulation is used, however, the return path can be connected to the remote ground connection (case) of implantable circuit 167, providing for much larger intermediate tissue for “Unipolar” stimulation. The “Bipolar” stimulation offers localized stimulation of tissue compared to “Unipolar” stimulation and is therefore, preferred in this embodiment. Unipolar stimulation is more likely to stimulate skeletal muscle in addition to nerve stimulation. The implanted circuit 167 in this embodiment is passive, so a battery does not have to be implanted.

The circuitry shown in FIGS. 11A and 11B can be used as an alternative for the implanted stimulus-receiver circuitry. The circuitry of FIG. 11A is a slightly simpler version, and circuitry of FIG. 11B contains a conventional NPN transistor 168 connected in an emitter-follower configuration.

For therapy to commence, the primary (external) coil 46 is placed on the skin 60 on top of the surgically implanted (secondary) coil 48. An adhesive tape is then placed on the skin 60 and external coil 46, such that the external coil 46 is taped to the skin 60. Other methods of attachment known in the art may also be used. For efficient energy transfer to occur, it is important that the primary (external) and secondary (internal) coils 46,48 be positioned along the same axis and be optimally positioned relative to each other. In this embodiment, the external coil 46 may be connected to proximity sensing circuitry 50. The correct positioning of the external coil 46 with respect to the internal coil 48 is indicated by turning “on” of a light emitting diode (LED) on the external stimulator 42.

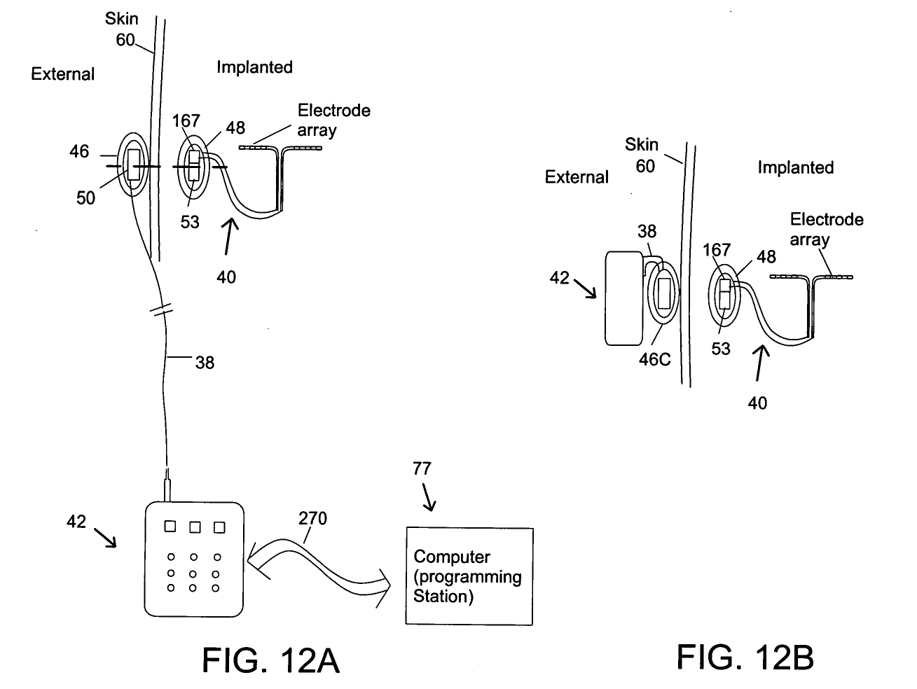

Optimal placement of the external (primary) coil 46 is done with the aid of proximity sensing circuitry incorporated in the system, in this embodiment. Proximity sensing occurs utilizing a combination of external and implantable components. The implanted components contains a relatively small magnet composed of materials that exhibit Giant Magneto-Resistor (GMR) characteristics such as Samarium-cobalt, a coil, and passive circuitry. Shown in conjunction with FIGS. 12A and 12B, the external coil 46 and proximity sensor circuitry 50 are rigidly connected in a convenient enclosure which is attached externally on the skin. The sensors measure the direction of the field applied from the magnet to sensors within a specific range of field strength magnitude. The dual sensors exhibit accurate sensing under relatively large separation between the sensor and the target magnet. As the external coil 46 placement is “fine tuned”, the condition where the external (primary) coil 46 comes in optimal position, i.e. is located adjacent and parallel to the subcutaneous (secondary) coil 48, along its axis, is recorded and indicated by a light emitting diode (LED) on the external stimulator 42.

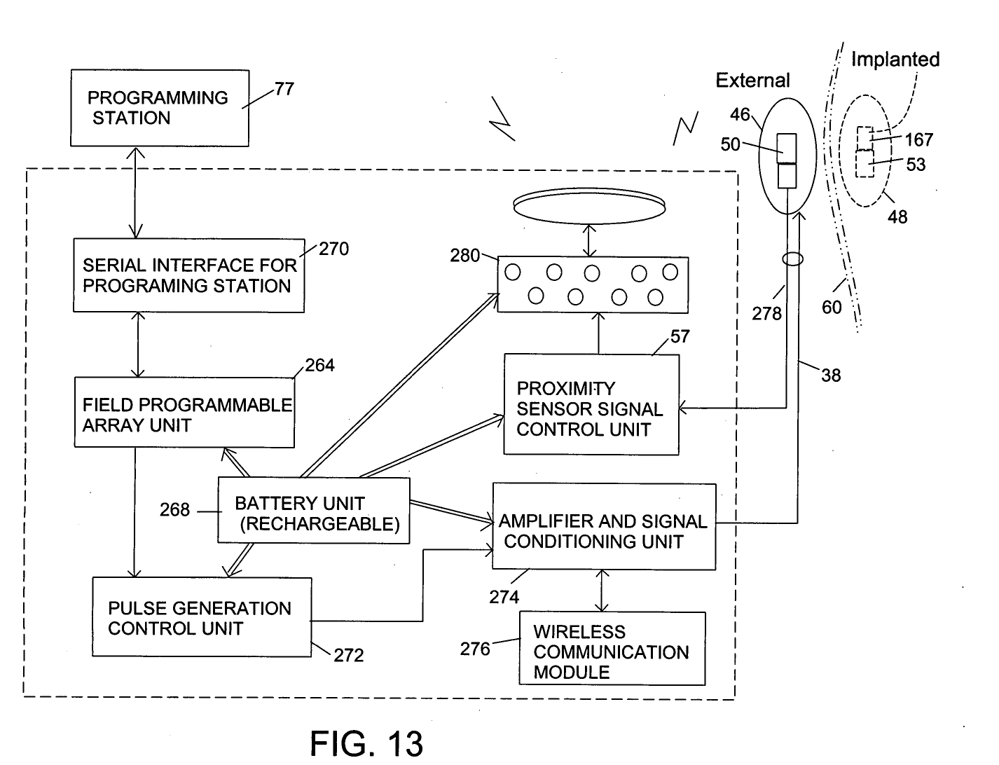

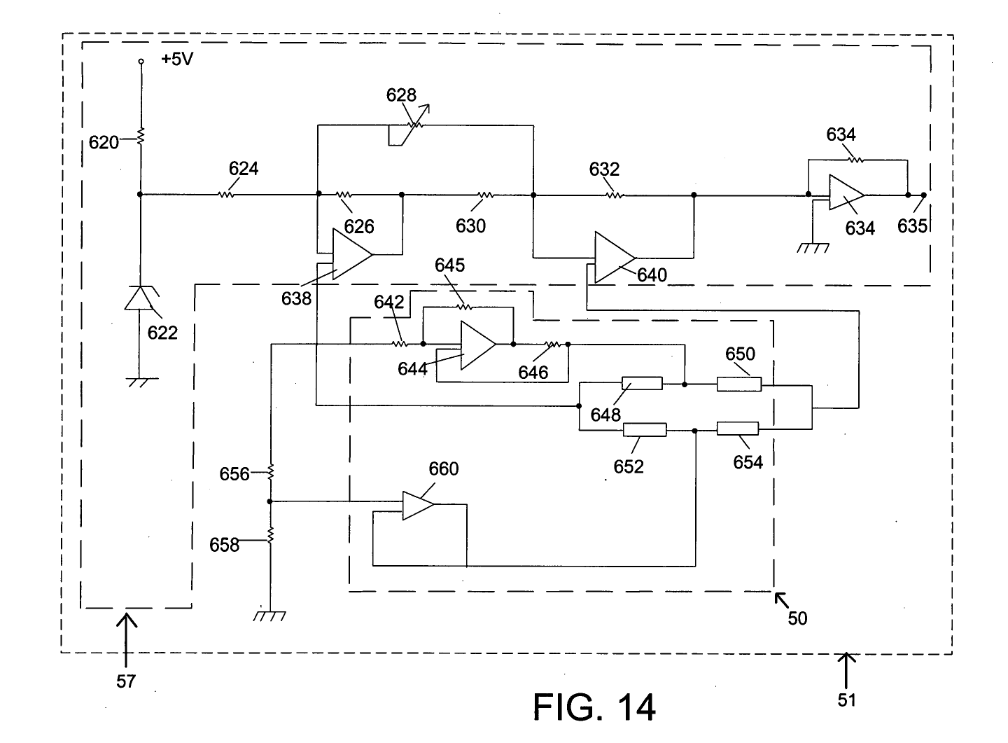

FIG. 13 shows an overall block diagram of the components of the external stimulator and the proximity sensing mechanism. The proximity sensing components are the primary (external) coil 46, supercutaneous (external) proximity sensors 648, 652 (FIG. 14) in the proximity sensor circuit unit 50, and a subcutaneous secondary coil 48 with a Giant Magneto Resister (GMR) magnet 53 associated with the proximity sensor unit. The proximity sensor circuit 50 provides a measure of the position of the secondary implanted coil 48. The signal output from proximity sensor circuit 50 is derived from the relative location of the primary and secondary coils 46, 48. The sub-assemblies consist of the coil and the associated electronic components, that are rigidly connected to the coil.

The proximity sensors (external) contained in the proximity sensor circuit 50 detect the presence of a GMR magnet 53, composed of Samarium Cobalt, that is rigidly attached to the implanted secondary coil 48. The proximity sensors, are mounted externally as a rigid assembly and sense the actual separation between the coils, also known as the proximity distance. In the event that the distance exceeds the system limit, the signal drops off and an alarm sounds to indicate failure of the production of adequate signal in the secondary implanted circuit 167, as applied in this embodiment of the device. This signal is provided to the location indicator LED 280.

FIG. 14 shows the circuit used to drive the proximity sensors 648, 652 of the proximity sensor circuit 50. The two proximity sensors 648, 652 obtain a proximity signal based on their position with respect to the implanted GMR magnet 53. This circuit also provides temperature compensation. The sensors 648, 652 are ‘Giant Magneto Resistor’ (GMR) type sensors packaged as proximity sensor unit 50. There are two components of the complete proximity sensor circuit. One component is mounted supercutaneously 50, and the other component, the proximity sensor signal control unit 57 is within the external stimulator 42. The resistance effect depends on the combination of the soft magnetic layer of magnet 53, where the change of direction of magnetization from external source can be large, and the hard magnetic layer, where the direction of magnetization remains unchanged. The resistance of this sensor 50 varies along a straight motion through the curvature of the magnetic field. A bridge differential voltage is suitably amplified and used as the proximity signal.

The Siemens GMR B6 (Siemens Corp., Special Components Inc., New Jersey) is used for this function in one embodiment. The maximum value of the peak-to-peak signal is observed as the external magnetic field becomes strong enough, at which point the resistance increases, resulting in the increase of the field-angle between the soft magnetic and hard magnetic material. The bridge voltage also increases. In this application, the two sensors 648, 652 are oriented orthogonal to each other.

The distance between the magnet 53 and sensor 50 is not relevant as long as the magnetic field is between 5 and 15 KA/m, and provides a range of distances between the sensors 648, 652 and the magnetic material 53. The GMR sensor registers the direction of the external magnetic field. A typical magnet to induce permanent magnetic field is approximately 15 by 8 by 5 mm³, for this application and these components. The sensors 648, 652 are sensitive to temperature, such that the corresponding resistance drops as temperature increases. This effect is quite minimal until about 100° C. A full bridge circuit is used for temperature compensation, as shown in temperature compensation circuit 50 of FIG. 14. The sensors 648, 652 and a pair of resistors 650, 654 are shown as part of the bridge network for temperature compensation. It is also possible to use a full bridge network of two additional sensors in place of the resistors 650, 654.

The signal from either proximity sensor 648, 652 is rectangular if the surface of the magnetic material is normal to the sensor and is radial to the axis of a circular GMR device. This indicates a shearing motion between the sensor and the magnetic device. When the sensor is parallel to the vertical axis of this device, there is a fall off of the relatively constant signal at about 25 mm. separation. The GMR sensor combination varies its resistance according to the direction of the external magnetic field, thereby providing an absolute angle sensor. The position of the GMR magnet can be registered at any angle from 0 to 360 degrees.

In the external stimulator 42 shown in FIG. 13, an indicator unit 280 which is provided to indicate proximity distance or coil proximity failure (for situations where the patch containing the external coil 46, has been removed, or is twisted abnormally etc.). Indication is also provided to assist in the placement of the patch. In case of general failure, a red light with audible signal is provided when the signal is not reaching the subcutaneous circuit. The indicator unit 280 also displays low battery status. The information on the low battery, normal and out of power conditions forewarns the user of the requirements of any corrective actions.

Also shown in FIG. 13, the programmable parameters are stored in a programmable logic 264. The predetermined programs stored in the external stimulator are capable of being modified through the use of a separate programming station 77. The Programmable Array Logic Unit 264 and interface unit 270 are interfaced to the programming station 77. The programming station 77 can be used to load new programs, change the existing predetermined programs or the program parameters for various stimulation programs. The programming station is connected to the programmable array unit 75 (comprising programmable array logic 304 and interface unit 270) with an RS232-C serial connection. The main purpose of the serial line interface is to provide an RS232-C standard interface. Other suitable connectors such as a USB connector or other connectors with standard protocols may also be used.

This method enables any portable computer with a serial interface to communicate and program the parameters for storing the various programs. The serial communication interface receives the serial data, buffers this data and converts it to a 16 bit parallel data. The programmable array logic 264 component of programmable array unit receives the parallel data bus and stores or modifies the data into a random access matrix. This array of data also contains special logic and instructions along with the actual data. These special instructions also provide an algorithm for storing, updating and retrieving the parameters from long-term memory. The programmable logic array unit 264, interfaces with long term memory to store the predetermined programs. All the previously modified programs can be stored here for access at any time, as well as, additional programs can be locked out for the patient. The programs consist of specific parameters and each unique program will be stored sequentially in long-term memory. A battery unit is present to provide power to all the components. The logic for the storage and decoding is stored in a random addressable storage matrix (RASM).

Conventional microprocessor and integrated circuits are used for the logic, control and timing circuits. Conventional bipolar transistors are used in radio-frequency oscillator, pulse amplitude ramp control and power amplifier. A standard voltage regulator is used in low-voltage detector. The hardware and software to deliver the predetermined programs is well known to those skilled in the art.

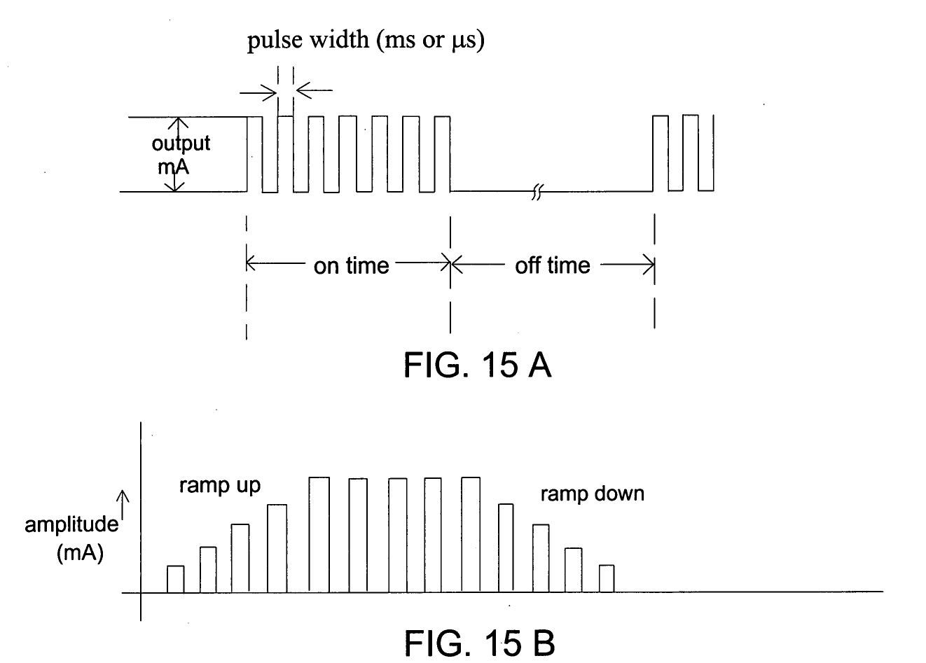

The pulses delivered to the nerve tissue for stimulation/blocking therapy are shown graphically in FIG. 15A. As shown in FIG. 15B, for patient comfort when the electrical stimulation is turned on, the electrical stimulation is ramped up and ramped down, instead of abrupt delivery of electrical pulses.

The selective stimulation to the occipital nerves can be performed in one of two ways. One method is to activate one of several “predetermined/pre-packaged” programs. A second method is to “custom” program the electrical parameters which can be selectively programmed, for specific therapy to the individual patient. The electrical parameters which can be individually programmed, include variables such as pulse amplitude, pulse width, frequency of stimulation, stimulation on-time, stimulation off-time, and electrode pair selection for stimulation. Table one below defines the approximate range of parameters:

TABLE 1
Electrical parameter range delivered to the nerve

| PARAMETER | RANGE |
|---|---|
| Pulse Amplitude | 0.1 Volt-15 Volts |
| Pulse width | 20 μS-5 mSec. |
| Stim. Frequency | 5 Hz-200 Hz |
| Freq. for blocking | DC to 750 Hz |
| On-time | 5 Secs-24 hours |
| Off-time | 5 Secs-24 hours |
| Electrode pairs | 1-2, 1-3, 1-4, |
| | 2-3, 2-4 |

The parameters in Table 3 are the electrical signals delivered to the occipital nerves via an electrode pair adjacent to the nerves. It being understood that the signals generated by the external pulse generator 42 and transmitted via the primary coil 46 are larger, because the attenuation factor between the primary coil 46 and secondary coil 48 is approximately 10-20 times, depending upon the distance, and orientation between the two coils. Accordingly, the range of transmitted signals of the external pulse generator are approximately 10-20 times larger than shown in Table 1.

Applicant's other patent disclosures also describe inductively coupled and implantable stimulation systems, which are listed below, and are incorporated herein by reference.

| Patent no. & date | Title: |
|---|---|
| 6,205,359 | Apparatus and method for adjunct (add-on) therapy |
| Mar. 20, 2001 | of partial complex epilepsy, generalized epilepsy |
| | and involuntary movement disorders utilizing an |
| | external stimulator. |
| 6,208,902 | Apparatus and method for adjunct (add-on) therapy |
| Mar. 27, 2001 | for pain syndromes utilizing an implantable lead |
| | and an external stimulator. |
| 6,662,052 | Method and system for neuromodulation therapy |
| Dec. 9, 2003 | using an external stimulator with wireless |
| | communication capabilities. |
| Jul. 16, 2002 | Method and system for modulating the vagus nerve |
| 10/196,533 | (10th cranial nerve) using modulated electrical |
| | pulses with an inductively coupled stimulation |
| | system. |
| May 11, 2003 | Method and system for providing pulsed electrical |
| 10/436,017 | stimulation to a cranial nerve of a patient to |
| | provide therapy for neurological and neuro- |
| | psychiatric disorders. |
| 6,473,652 | Method and apparatus for locating implanted |
| Oct. 29, 2002 | receiver and feedback regulation between |
| | subcutaneous and external coils. |
| 6,760,626 | Apparatus and method for treatment of neurological |
| Jul. 6, 2004 | and neuropsychiatric disorders using programmer- |
| | less implantable pulse generator system. |

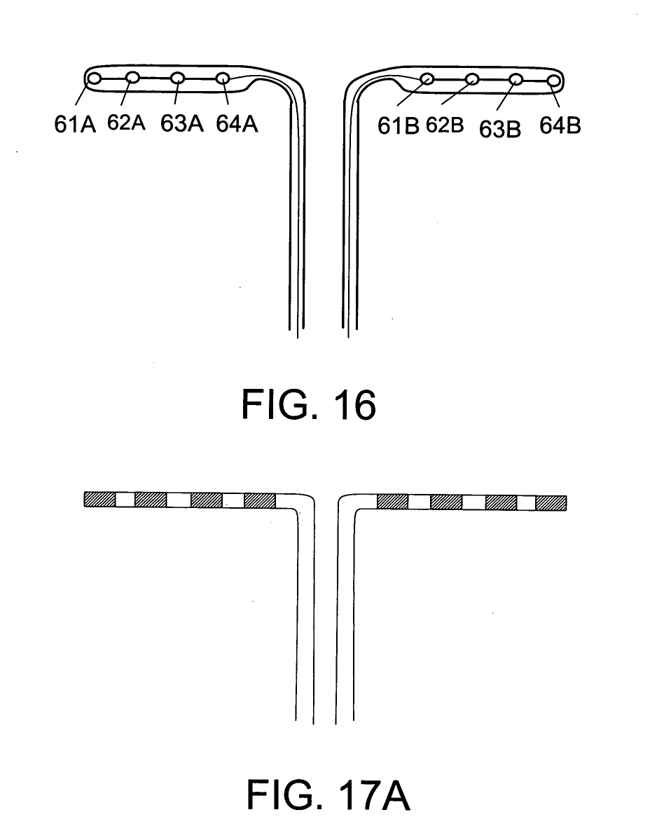

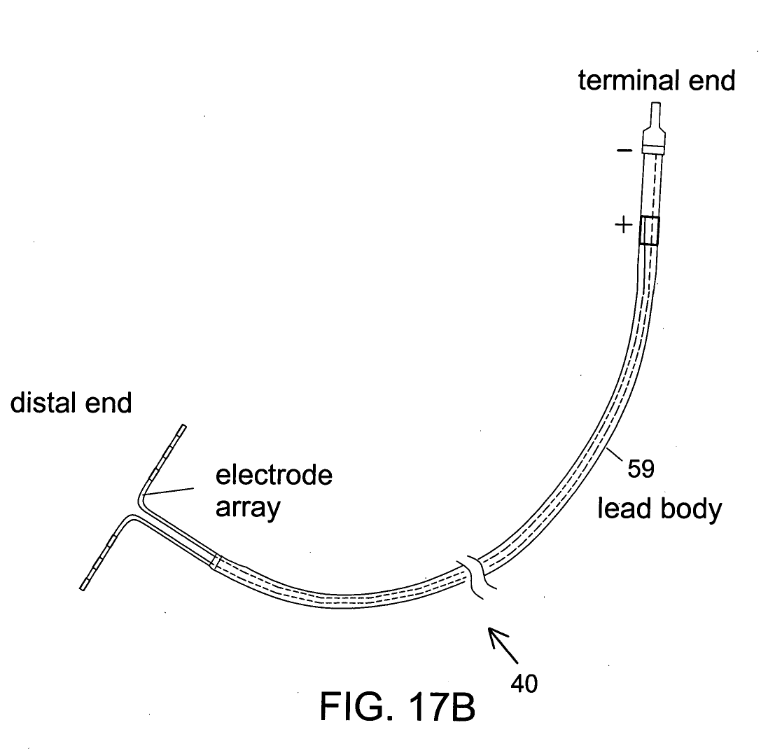

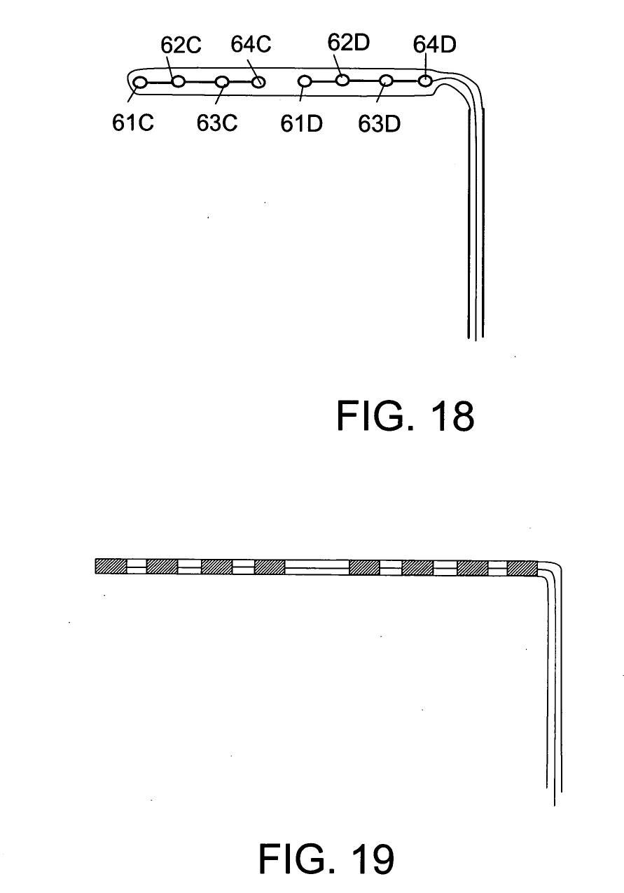

FIGS. 16, 17A, 17B, 18, and 19 show examples of leads. The multiple electrodes (electrode array) may be on a lead or a lead pair. For implanting a lead pair, a midline incision is generally used, and a lateral incision is generally used for implanting a single lead. The electrode pair used for stimulation may vary, and is a programmable parameter. For reasons of better clinical efficacy, the preferred embodiment utilizes a pair of paddle leads as shown in FIG. 16. Alternatively, a pair of cylindrical leads may also be utilized, as is shown in conjunction with FIG. 17A. Single paddle lead shown in FIG. 18, and single cylindrical lead, shown in FIG. 19, may also be utilized. In this embodiment, single lead comprises eight electrodes, and a lead pair also comprises 8 electrodes with 4 electrodes per lead. It will be clear to one skilled in the art that larger or smaller number of electrodes may also be utilized, and such is considered within the scope of the invention.

The lead terminal preferably is linear bipolar, even though it can be bifurcated, and plug(s) into the cavity of the pulse generator means. The lead body 59 insulation may be constructed of medical grade silicone, silicone reinforced with polytetrafluoro-ethylene (PTFE), or polyurethane. The electrodes for stimulating the occipital nerves may be button electrodes or may be cylindrical electrodes. These stimulating electrodes may be made of pure platinum, platinum/Iridium alloy or platinum/iridium coated with titanium nitride. The conductor connecting the terminal to the electrodes is made of an alloy of nickel-cobalt. The implanted lead design variables are also summarized in table two below.

TABLE 2
Lead design variables

| | | | Conductor | | |
|---|---|---|---|---|---|
| Proximal | | | (connecting | | Distal |
| End | Lead body- | | proximal | | End |
| Lead | Insulation | | and distal | Electrode - | Electrode - |
| Terminal | Materials | Lead-Coating | ends) | Material | Type |
| Linear | Polyurethane | Antimicrobial | Alloy of | Pure | Button |
| bipolar | | coating | Nickel- | Platinum | electrodes |
| | | | Cobalt | | |
| Bifurcated | Silicone | Anti- | | Platinum- | Cylindrical |
| | | Inflammatory | | Iridium | electrodes |
| | | coating | | (Pt/Ir) Alloy | |
| | Silicone with | | | Pt/Ir coated | Drug- |
| | Polytetrafluoro- | | | with Titanium | eluting |
| | ethylene | | | Nitride | electrode |
| | (PTFE) | | | | |
| | | | | Carbon | |

Once the lead is fabricated, coating such as anti-microbial, anti-inflammatory, or lubricious coating may be applied to the body of the lead.

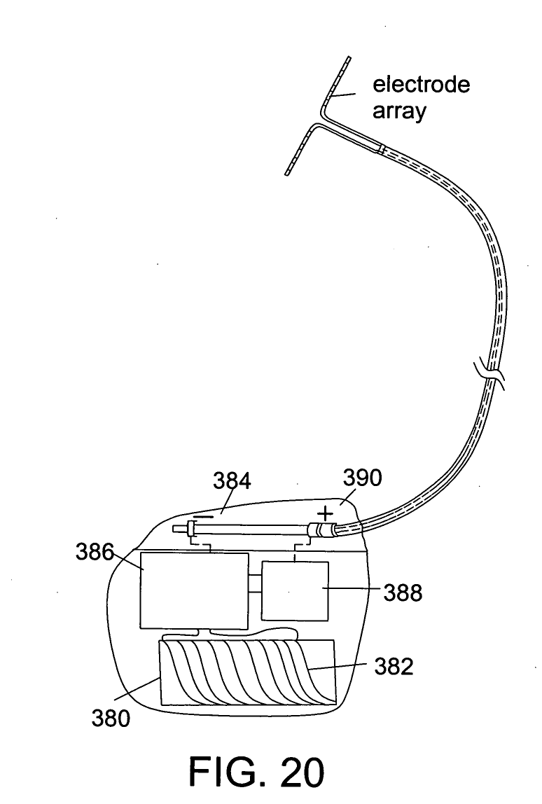

Implanted stimulus-receiver comprising a high value capacitor for storing charge, used in conjunction with an external stimulator Another embodiment using the same principles is described schematically in FIGS. 20, 21 and 22. Using mostly hybrid components and appropriate packaging, the implanted portion of the system described below is conducive to miniaturization. As shown in FIG. 20, a solenoid coil 382 wrapped around a ferrite core 380 is used as the secondary of an air gap transformer for receiving power and data to the implanted device 34. The primary coil is external to the body. Since the coupling between the external transmitter coil 367 and receiver coil 382 may be weak, a high-efficiency transmitter/amplifier is used in order to supply enough power to the receiver coil 382. Class-D or Class-E power amplifiers may be used for this purpose, and are described later.

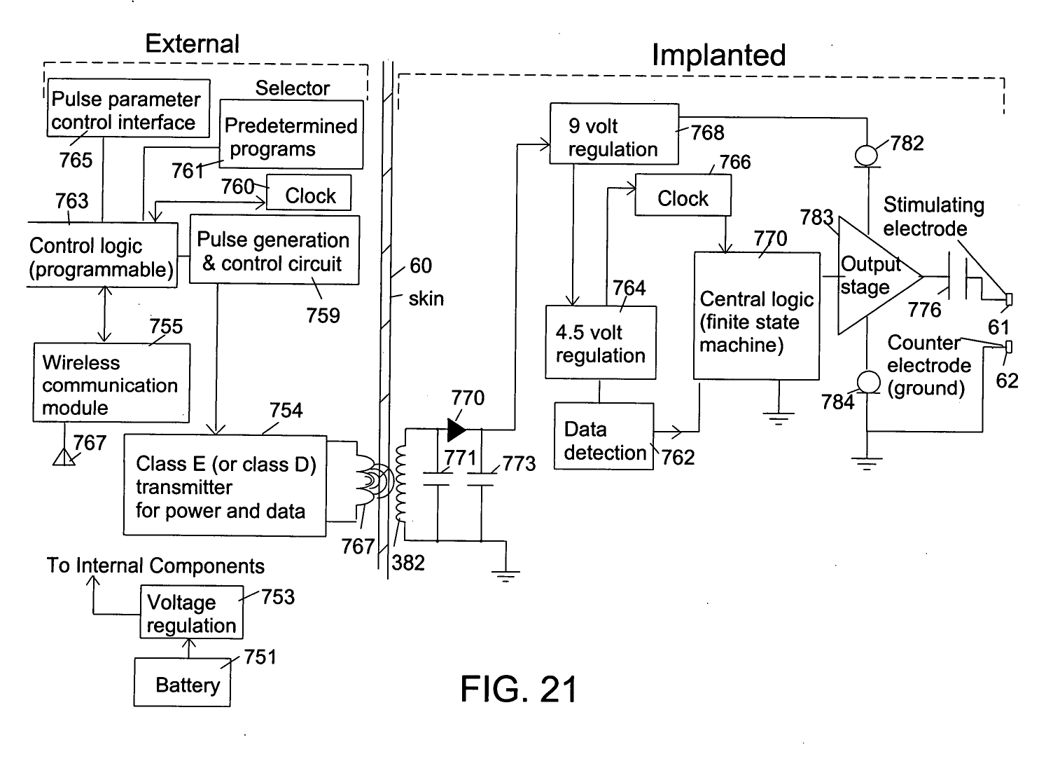

As shown in conjunction with FIG. 21, the received signal after being picked by the resonant tank circuit comprising of inductor 382 and capacitor 771, goes through a rectifier 770. Even though a single diode 770 is shown in the figure, a diode bridge can be used for full-wave rectification, and the signal then goes through two series voltage regulators in order to generate the required supply voltages. The voltage regulators consist of rectifier, storage capacitor, and 4.5-V and 9-V shunt regulators implemented using Zenor diodes and resistors (not shown in FIG. 21). Bipolar transistors and diodes with high breakdown voltages are used to provide protection from high input voltages. Clock 766 is regenerated from the radio-frequency (RF) carrier by taking the peak amplitude of sinusoidal carrier input and generating a 4.5-V square wave output. Data detection circuitry is comprised using a low-pass filter (LPF), a high-pass filter (HPF), and a Schmitt trigger for envelope detection and noise suppression. The low-pass filter is necessary in order to extract the envelope from the high frequency carrier. Finally, the output circuit contains charge-balance circuitry, stimulus current regulator circuitry, and startup circuitry. As also shown in FIG. 21, a Class-D or Class E driver can be used in the external transmitter.

In one embodiment, the implanted stimulus-receiver may be a system which is RF coupled combined with a power source. In this embodiment, the implanted stimulus-receiver comprises high value, small sized capacitor(s) for storing charge and delivering electric stimulation pulses for up to several hours by itself, once the capacitors are charged. The packaging is shown in FIG. 20. Using mostly hybrid components and appropriate packaging, the implanted portion of the system described below is conducive to miniaturization. As shown in FIG. 20, a solenoid coil 382 wrapped around a ferrite core 380 is used as the secondary of an air-gap transformer for receiving power and data to the implanted device. The primary coil is external to the body. Since the coupling between the external transmitter coil and receiver coil 382 may be weak, a high-efficiency transmitter/amplifier is used in order to supply enough power to the receiver coil 382. Class-D or Class-E power amplifiers may be used for this purpose. The coil for the external transmitter (primary coil) may be placed in the pocket of a customized garment.

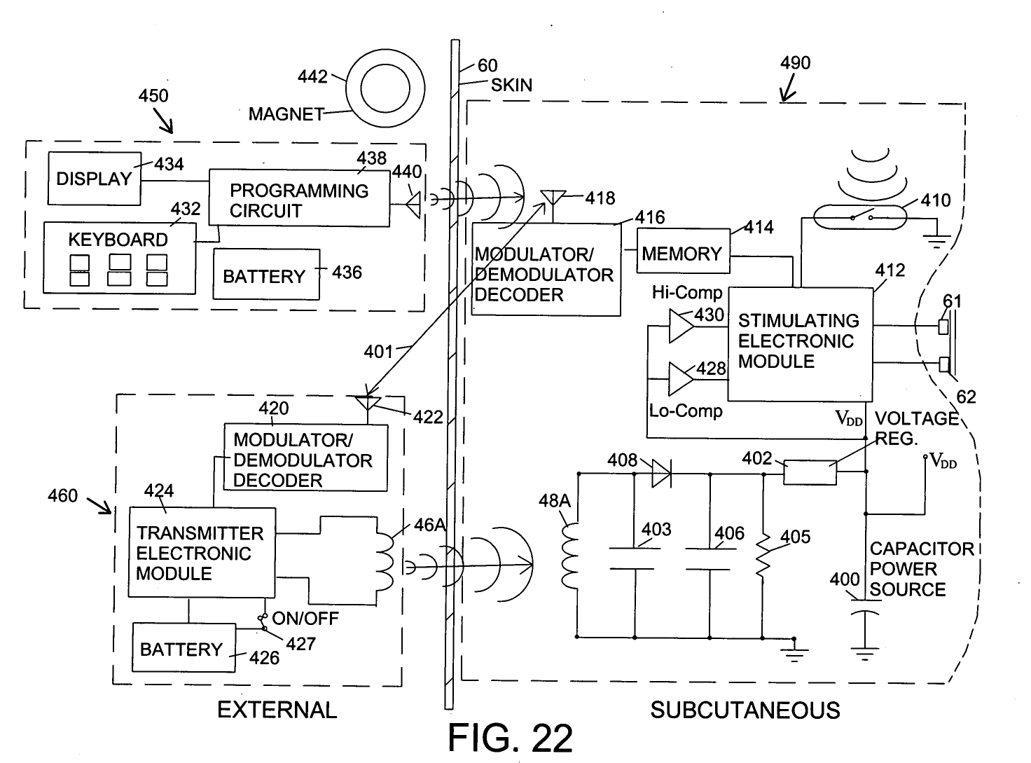

In this embodiment, as shown in conjunction with FIG. 22 of the implanted stimulus-receiver 490 and the system, the receiving inductor 48A and tuning capacitor 403 are tuned to the frequency of the transmitter. The diode 408 rectifies the AC signals, and a small sized capacitor 406 is utilized for smoothing the input voltage V_{I} fed into the voltage regulator 402. The output voltage V_{D} of regulator 402 is applied to capacitive energy power supply and source 400 which establishes source power VDD. Capacitor 400 is a big value, small sized capacative energy source which is classified as low internal impedance, low power loss and high charge rate capacitor, such as Panasonic Model No. 641.

The refresh-recharge transmitter unit 460 includes a primary battery 426, an ON/Off switch 427, a transmitter electronic module 442, an RF inductor power coil 46A, a modulator/demodulator 420 and an antenna 422.

When the ON/OFF switch is on, the primary coil 46A is placed in close proximity to skin 60 and secondary coil 48A of the implanted stimulator 490. The inductor coil 46A emits RF waves establishing EMF wave fronts which are received by secondary inductor 48A. Further, transmitter electronic module 442 sends out command signals which are converted by modulator/demodulator decoder 420 and sent via antenna 422 to antenna 418 in the implanted stimulator 490. These received command signals are demodulated by decoder 416 and replied and responded to, based on a program in memory 414 (matched against a “command table” in the memory). Memory 414 then activates the proper controls and the inductor receiver coil 48A accepts the RF coupled power from inductor 46A.

The RF coupled power, which is alternating or AC in nature, is converted by the rectifier 408 into a high DC voltage. Small value capacitor 406 operates to filter and level this high DC voltage at a certain level. Voltage regulator 402 converts the high DC voltage to a lower precise DC voltage while capacitive power source 400 refreshes and replenishes.

When the voltage in capacative source 400 reaches a predetermined level (that is VDD reaches a certain predetermined high level), the high threshold comparator 430 fires and stimulating electronic module 412 sends an appropriate command signal to modulator/decoder 416. Modulator/decoder 416 then sends an appropriate “fully charged” signal indicating that capacitive power source 400 is fully charged, is received by antenna 422 in the refresh-recharge transmitter unit 460.

In one mode of operation, the patient may start or stop stimulation by waving the magnet 442 once near the implant. The magnet emits a magnetic force Lm which pulls reed switch 410 closed. Upon closure of reed switch 410, stimulating electronic module 412 in conjunction with memory 414 begins the delivery (or cessation as the case may be) of controlled electronic stimulation pulses to the occipital nerves via electrodes 61, 62. In another mode (AUTO), the stimulation is automatically delivered to the implanted lead based upon programmed ON/OFF times.

The programmer unit 450 includes keyboard 432, programming circuit 438, rechargeable battery 436, and display 434. The physician or medical technician programs programming unit 450 via keyboard 432. This program regarding the frequency, pulse width, modulation program, ON time etc. is stored in programming circuit 438. The programming unit 450 must be placed relatively close to the implanted stimulator 490 in order to transfer the commands and programming information from antenna 440 to antenna 418. Upon receipt of this programming data, modulator/demodulator and decoder 416 decodes and conditions these signals, and the digital programming information is captured by memory 414. This digital programming information is further processed by stimulating electronic module 412. In the DEMAND operating mode, after programming the implanted stimulator, the patient turns ON and OFF the implanted stimulator via hand held magnet 442 and the reed switch 410. In the automatic mode (AUTO), the implanted stimulator turns ON and OFF automatically according to the programmed values for the ON and OFF times.

Other simplified versions of such a system may also be used. For example, a system such as this, where a separate programmer is eliminated, and simplified programming is performed with a magnet and reed switch, can also be used.

Programmer-Less Implantable Pulse Generator (IPG)

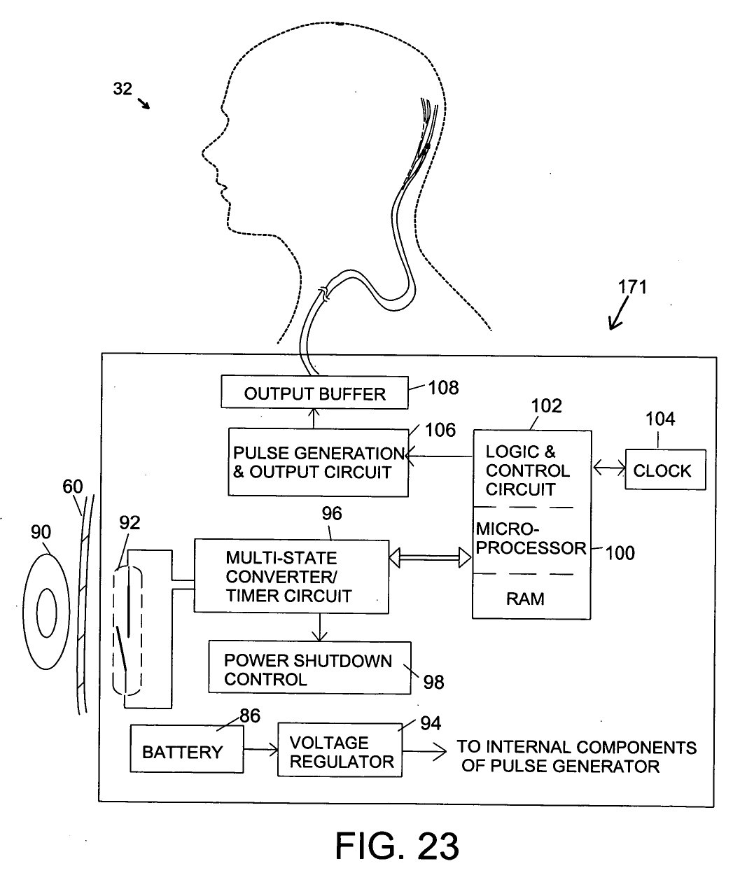

In one embodiment, a programmer-less implantable pulse generator (IPG) may be used, as disclosed in applicant's commonly assigned U.S. Pat. No. 6,760,626 B1, which is incorporated herein by reference. In this embodiment, shown in conjunction with FIG. 23, the implantable pulse generator 171 is provided with a reed switch 92 and memory circuitry 102. The reed switch 92 being remotely actuable by means of a magnet 90 brought into proximity of the pulse generator 171, in accordance with common practice in the art. In this embodiment, the reed switch 92 is coupled to a multi-state converter/timer circuit 96, such that a single short closure of the reed switch can be used as a means for non-invasive encoding and programming of the pulse generator 171 parameters.

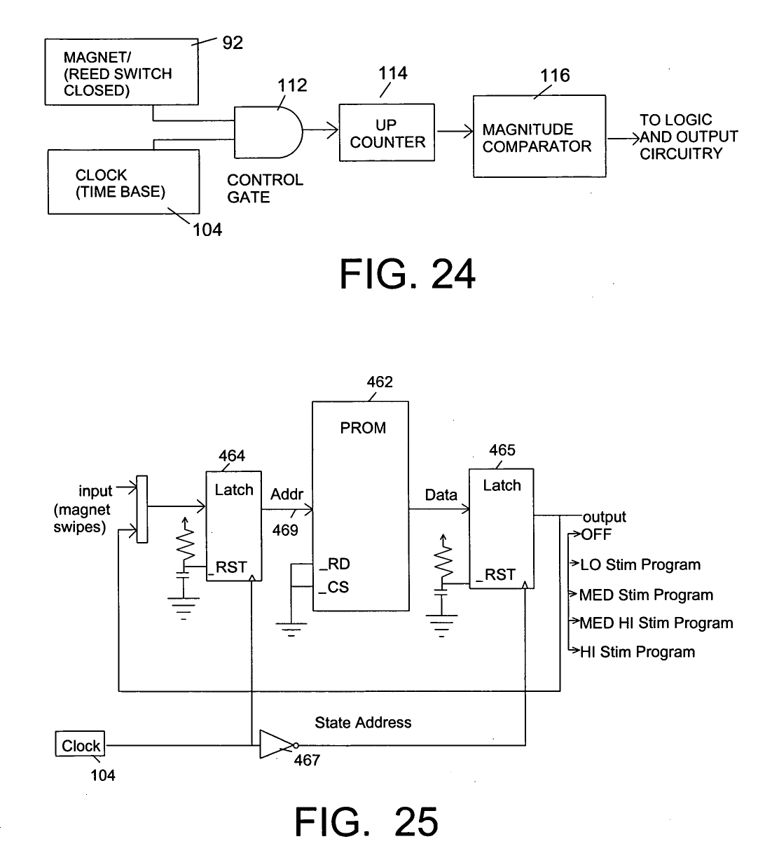

In one embodiment, shown in conjunction with FIG. 24, the closing of the reed switch 92 triggers a counter. The magnet 90 and timer are ANDed together. The system is configured such that during the time that the magnet 82 is held over the pulse generator 171, the output level goes from LOW stimulation state to the next higher stimulation state every 5 seconds. Once the magnet 82 is removed, regardless of the state of stimulation, an application of the magnet, without holding it over the pulse generator 171, triggers the OFF state, which also resets the counter.

Once the prepackaged/predetermined logic state is activated by the logic and control circuit 102, as shown in FIG. 23, the pulse generation and amplification circuit 106 deliver the appropriate electrical pulses to the occipital nerves of the patient via an output buffer 108. The delivery of output pulses is configured such that the distal electrode 61 is the cathode and the proximal electrode 62 is the anode. Timing signals for the logic and control circuit 102 of the pulse generator 171 are provided by a crystal oscillator 104. The battery 86 of the pulse generator 171 has terminals connected to the input of a voltage regulator 94. The regulator 94 smoothes the battery output and supplies power to the internal components of the pulse generator 171. A microprocessor 100 controls the program parameters of the device, such as the voltage, pulse width, frequency of pulses, on-time and off-time. The microprocessor may be a commercially available, general purpose microprocessor or microcontroller, or may be a custom integrated circuit device augmented by standard RAM/ROM components.

In one embodiment, there are four stimulation states. A larger (or lower) number of states can be achieved using the same methodology, and such is considered within the scope of the invention. These four states are (without limitation), LOW stimulation state, LOW-MED stimulation state, MED stimulation state, and HIGH stimulation state. Examples of stimulation parameters (delivered to the occipital nerves) for each state are as follows,

LOW stimulation state example is,

| | | |
|---|---|---|
| Output amplitude: | 1.5 | volts. |
| Pulse width: | 0.20 | msec. |
| Pulse frequency: | 60 | Hz |
| Cycles: | 25 | sec. on-time and 1.5 min. off-time |
| | | in repeating cycles. |

LOW-MED stimulation state example is,

| | | |
|---|---|---|
| Output amplitude: | 2.5 | volts. |
| Pulse width: | 0.25 | msec. |
| Pulse frequency: | 70 | Hz |
| Cycles: | 20 | sec. on-time and 1 min. off-time |
| | | in repeating cycles. |

MED stimulation state example is,

| | | |
|---|---|---|
| Output amplitude: | 2.5 | volts. |
| Pulse width: | 0.30 | msec. |
| Pulse frequency: | 75 | Hz |
| Cycles: | 20 | sec. on-time and 50 sec. off-time |
| | | in repeating cycles. |

HIGH stimulation state example is,

| | | |
|---|---|---|
| Output amplitude: | 5.0 | volts. |
| Pulse width: | 0.40 | msec. |
| Pulse frequency: | 90 | Hz |
| Cycles: | 15 | sec. on-time and 30 sec. off-time |
| | | in repeating cycles |

These pre-packaged/predetermined programs are mearly examples, and the actual stimulation parameters will deviate from these depending on the treatment application.

It will be readily apparent to one skilled in the art, that other schemes can be used for the same purpose. For example, instead of placing the magnet 90 on the pulse generator 171 for a prolonged period of time, different stimulation states can be encoded by the sequence of magnet applications. Accordingly, in an alternative embodiment there can be three logic states, OFF, LOW stimulation (LS) state, and HIGH stimulation (HS) state. Each logic state again corresponds to a pre-packaged/predetermined program such as presented above. In such an embodiment, the system could be configured such that one application of the magnet triggers the generator into LS State. If the generator is already in the LS state then one application triggers the device into OFF State. Two successive magnet applications triggers the generator into MED stimulation state, and three successive magnet applications triggers the pulse generator in the HIGH Stimulation State. Subsequently, one application of the magnet while the device is in any stimulation state, triggers the device OFF.

FIG. 25 shows a representative digital circuitry used for the basic state machine circuit. The circuit consists of a PROM 462 that has part of its data fed back as a state address. Other address lines 469 are used as circuit inputs, and the state machine changes its state address on the basis of these inputs. The clock 104 is used to pass the new address to the PROM 462 and then pass the output from the PROM 462 to the outputs and input state circuits. The two latches 464, 465 are operated 180° out of phase to prevent glitches from unexpectedly affecting any output circuits when the ROM changes state. Each state responds differently according to the inputs it receives.

The advantage of this embodiment is that it is cheaper to manufacture than a fully programmable implantable pulse generator (IPG).

### Programmable Implantable Pulse Generator (IPG)

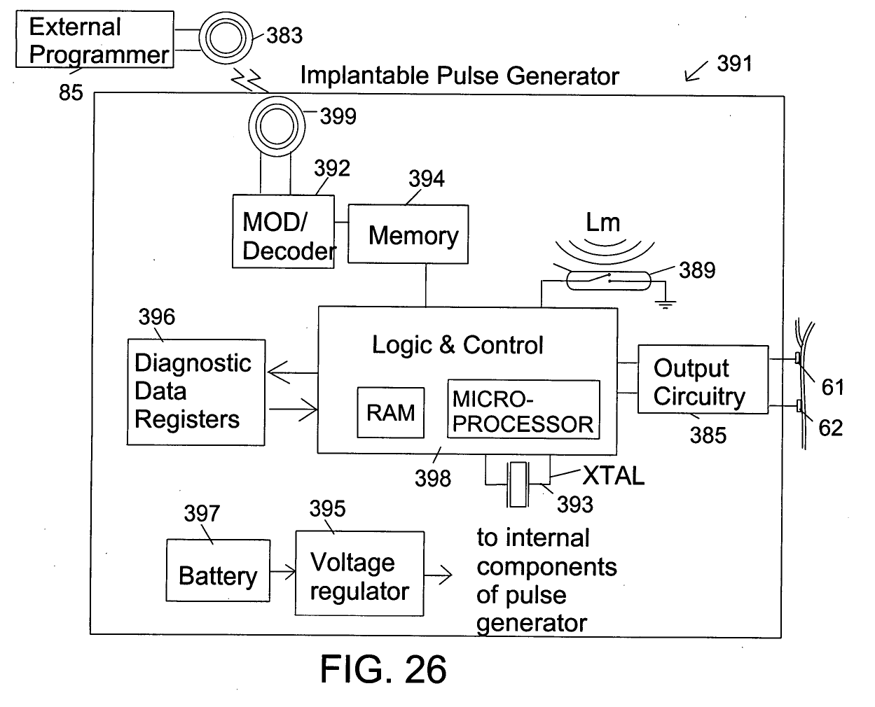

In one embodiment, a fully programmable implantable pulse generator (IPG), capable of generating stimulation and blocking pulses may be used. Shown in conjunction with FIG. 26, the implantable pulse generator unit 391 is preferably a microprocessor based device, where the entire circuitry is encased in a hermetically sealed titanium can. As shown in the overall block diagram, the logic & control unit 398 provides the proper timing for the output circuitry 385 to generate electrical pulses that are delivered to electrode pair in contact with the nerve tissue, via a lead 40. Programming of the implantable pulse generator (IPG) is done via an external programmer 85, as described later. Once activated or programmed via an external programmer 85, the implanted pulse generator 391 provides appropriate electrical stimulation pulses to the occipital nerves via an electrode pair.

This embodiment also comprises predetermined/pre-packaged programs. Examples of four stimulation states were given in the previous section, under “Programmer-less Implantable Pulse Generator (IPG)”. These predetermined/pre-packaged programs comprise unique combinations of pulse amplitude, pulse width, pulse morphology, pulse frequency, electrode pair selection, ON-time and OFF-time. Any number of predetermined/pre-packaged programs can be stored in the implantable pulse generator of this invention.

Examples of additional predetermined/pre-packaged programs are:

Program one:

| | |
|---|---|
| Output amplitude: | 1.5 volts. |
| Pulse width: | 0.20 msec. |
| Pulse frequency: | 60 Hz |
| Cycles: | 15 sec. on-time and 1.0 min. off-time in repeating |
| | cycles. |

Program two:

| | |
|---|---|
| Output amplitude: | 2.0 volts. |
| Pulse width: | 0.25 msec. |
| Pulse frequency: | 65 Hz |
| Cycles: | 15 sec. on-time and 50 sec. off-time in repeating |
| | cycles. |

Program three:

| | |
|---|---|
| Output amplitude: | 2.5 volts. |
| Pulse width: | 0.30 msec. |
| Pulse frequency: | 70 Hz |
| Cycles: | 20 sec. on-time and 1 min. off-time in repeating |
| | cycles. |

Program four:

| | |
|---|---|
| Output amplitude: | 3.0 volts. |
| Pulse width: | 0.35 msec. |
| Pulse frequency: | 75 Hz |
| Cycles: | 15 sec. on-time and 30 sec. off-time in repeating |
| | cycles. |
| Output amplitude: | 3.5 volts. |
| Pulse width: | 0.40 msec. |
| Pulse frequency: | 80 Hz |
| Cycles: | 20 sec. on-time and 40 sec. off-time in repeating |
| | cycles. |

Program six (fast cycle):

| | |
|---|---|
| Output amplitude: | 2.5 volts. |
| Pulse width: | 0.35 msec. |
| Pulse frequency: | 75 Hz |
| Cycles: | 20 sec. on-time and 30 sec. off-time in repeating |
| | cycles. |

Program seven (fast cycle):

| | |
|---|---|
| Output amplitude: | 3.5 volts. |
| Pulse width: | 0.4 msec. |
| Pulse frequency: | 85 Hz |
| Cycles: | 30 sec. on-time and 45 sec. off-time in repeating |
| | cycles. |

Program eight (complex pulses):

| | |
|---|---|
| Output amplitude: | 3.5 volts. |
| Pulse width: | 0.4 msec. |
| Pulse frequency: | 85 Hz |
| Pulse type: | step pulses |
| Cycles: | 20 sec. on-time and 3.0 min. off-time in repeating |
| | cycles. |
| Output amplitude: | 3.5 volts. |
| Pulse width: | 0.4 msec. |
| Pulse frequency: | 85 Hz |
| Pulse type: | step pulses |
| Cycles: | 20 sec. on-time and 3.0 min. off-time in repeating |
| | cycles. |

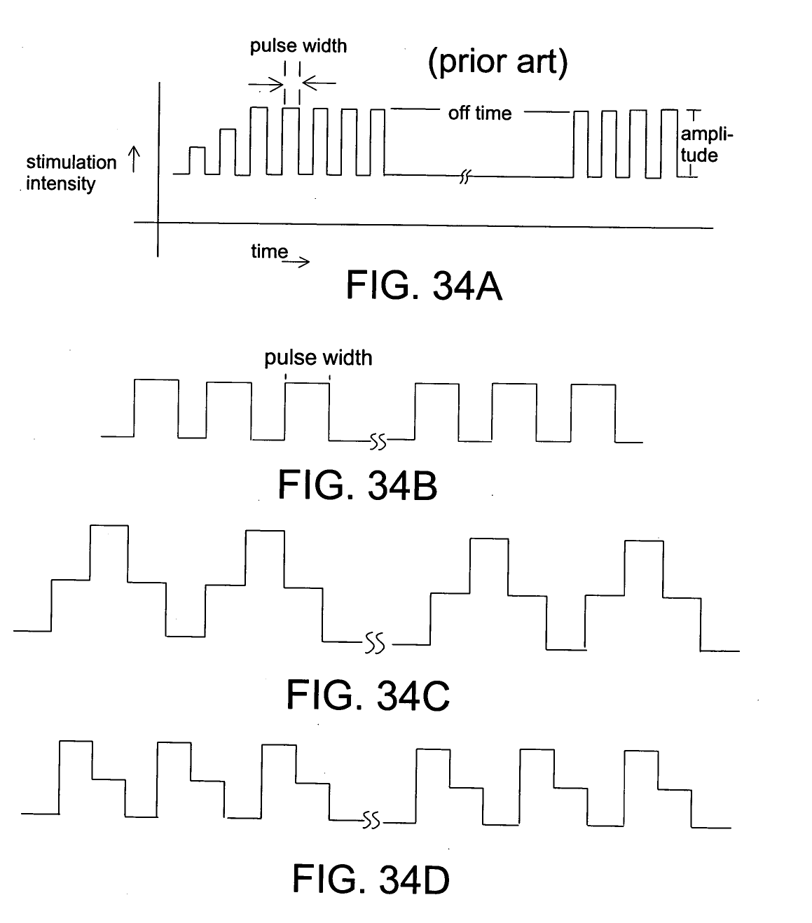

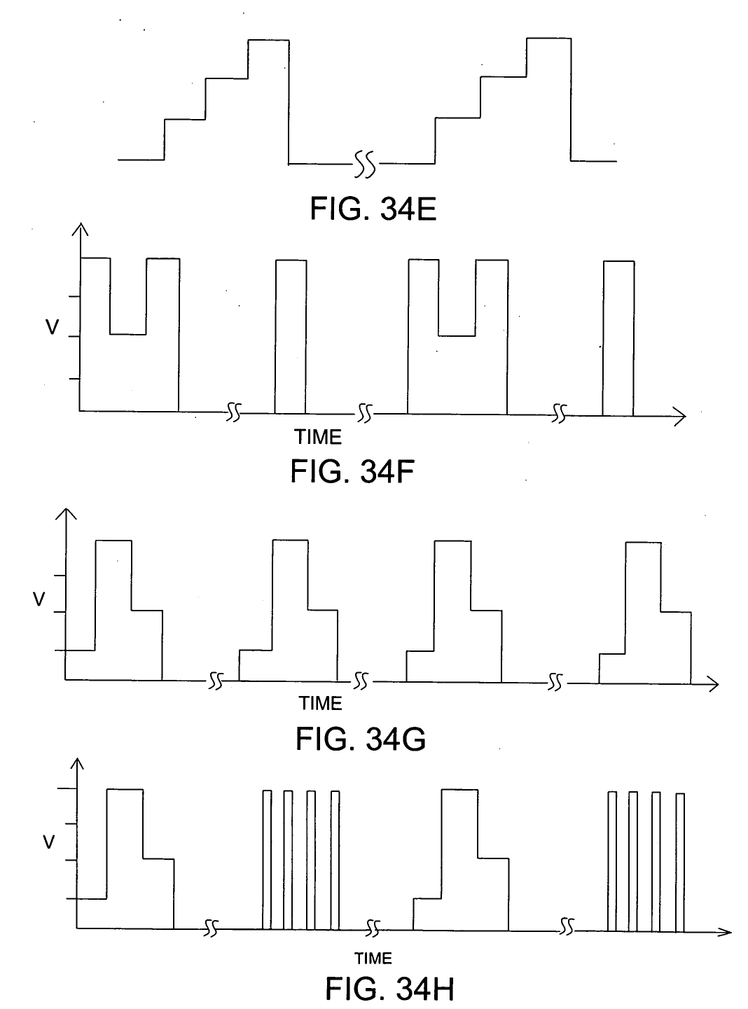

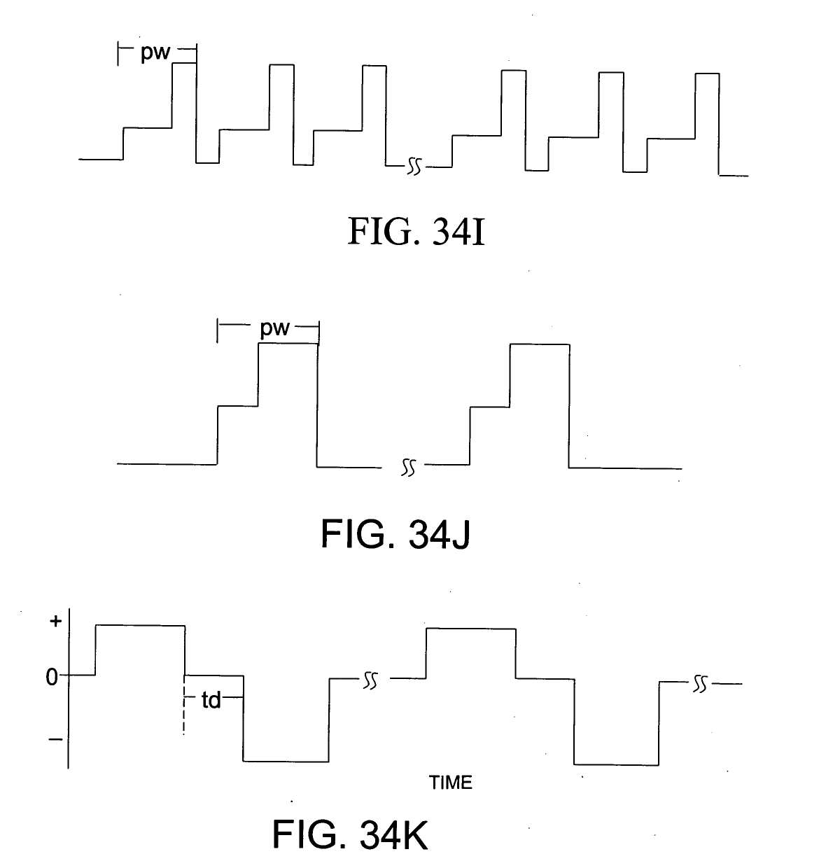

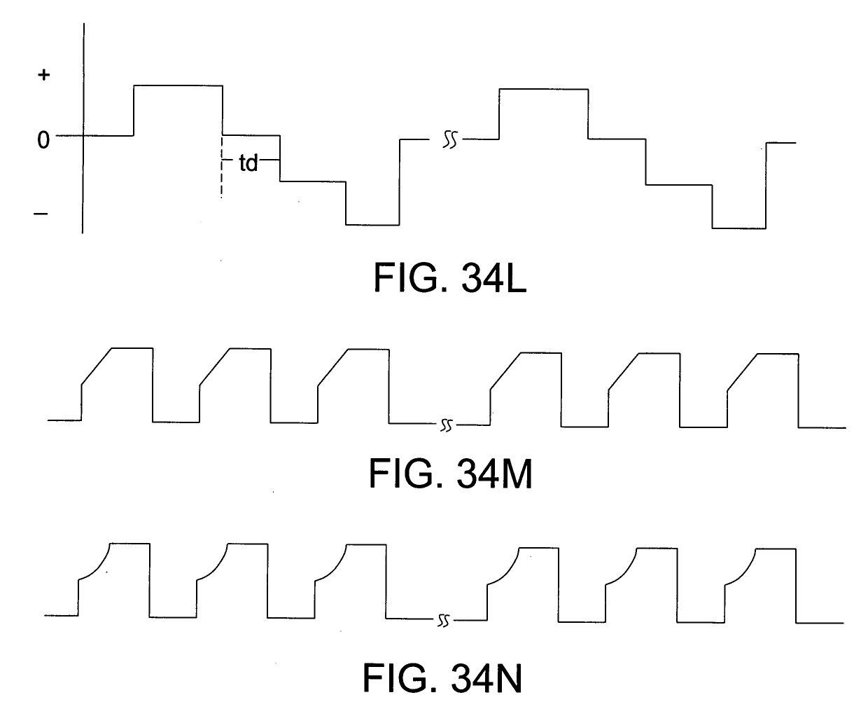

Program ten (complex pulse train):

| | |
|---|---|
| Output amplitude: | 3.5 volts. |
| Pulse width: | 0.4 msec. |
| Pulse frequency: | 85 Hz |
| Pulse type: | step pulses with alternating pulse train |
| | (as shown in FIG. 34H) |
| Cycles: | 20 sec. on-time and 3.0 min. off-time in repeating |
| | cycles. |

These pre-packaged/predetermined programs are mearly examples, and the actual stimulation parameters of the programs will deviate from these depending on the treatment application and physician preference. One advantage of predetermined/pre-packaged program is that it can be readily activated by a program number. A simple version of a programmer, adapted to activate only a limited number of predetermined/pre-packaged programs may also be supplied to the patient.

In addition, each parameter may be individually adjusted and stored in the memory 394. The range of programmable electrical stimulation parameters include both stimulating and blocking frequencies, and are shown in table three below.

TABLE 3
Programmable electrical parameter range

| PARAMER | RANGE |
|---|---|
| Pulse Amplitude | 0.1 Volt-15 Volts |
| Pulse width | 20 μS-5 mSec. |
| Stim. Frequency | 5 Hz-200 Hz |
| Freq. for blocking | DC to 750 Hz |
| On-time | 5 Secs-24 hours |
| Off-time | 5 Secs-24 hours |
| Ramp | ON/OFF |
| Electrode Pairs | 1-2, 1-3, 1-4, |
| | 2-3, 2-4 |

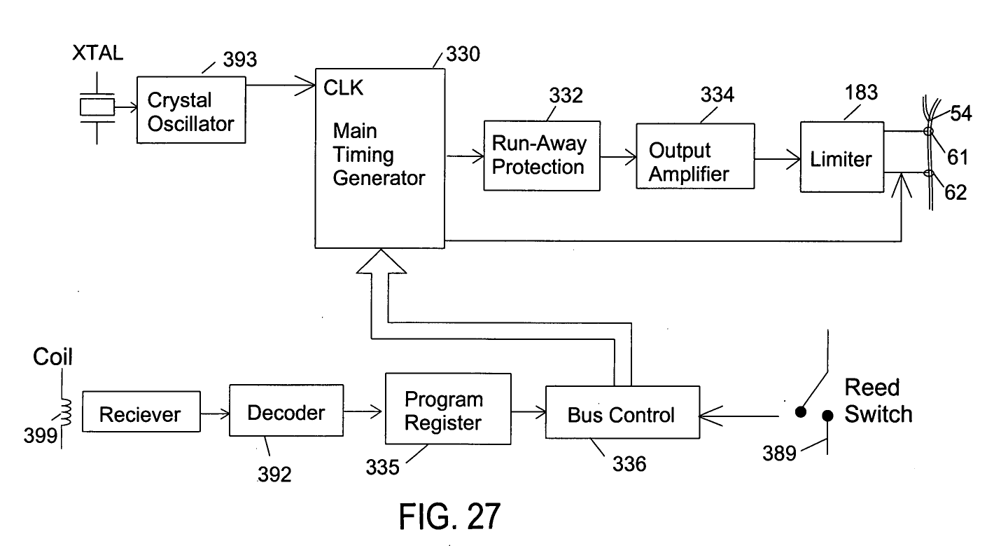

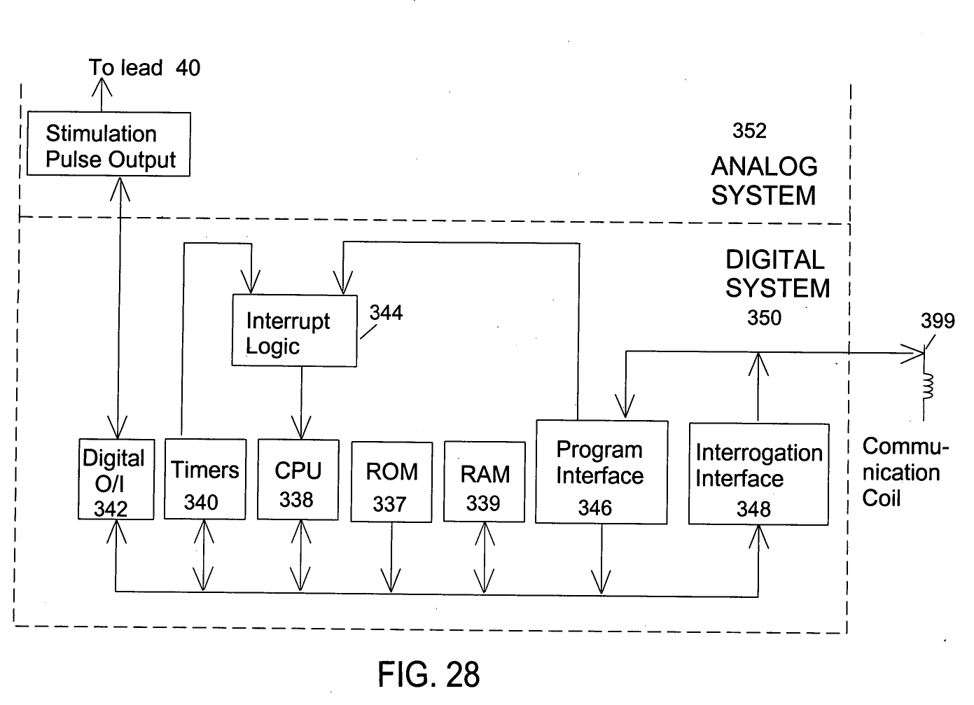

Shown in conjunction with FIGS. 27 and 28, the electronic stimulation module comprises both digital 350 and analog 352 circuits. A main timing generator 330 (shown in FIG. 27), controls the timing of the analog output circuitry for delivering neuromodulating pulses to the occipital nerves, via output amplifier 334. Limiter 183 prevents excessive stimulation energy from getting to the occipital nerves. The main timing generator 330 receiving clock pulses from crystal oscillator 393. Main timing generator 330 also receiving input from programmer 85 via coil 399. FIG. 28 highlights other portions of the digital system such as CPU 338, ROM 337, RAM 339, program interface 346, interrogation interface 348, timers 340, and digital O/I 342.

Most of the digital functional circuitry 350 is on a single chip (IC). This monolithic chip along with other IC's and components such as capacitors and the input protection diodes are assembled together on a hybrid circuit. As well known in the art, hybrid technology is used to establish the connections between the circuit and the other passive components. The integrated circuit is hermetically encapsulated in a chip carrier. A coil 399 situated under the hybrid substrate is used for bidirectional telemetry. The hybrid and battery 397 are encased in a titanium can 65. This housing is a two-part titanium capsule that is hermetically sealed by laser welding. Alternatively, electron-beam welding can also be used. The header 79 is a cast epoxy-resin with hermetically sealed feed-through, and form the lead 40 connection block.

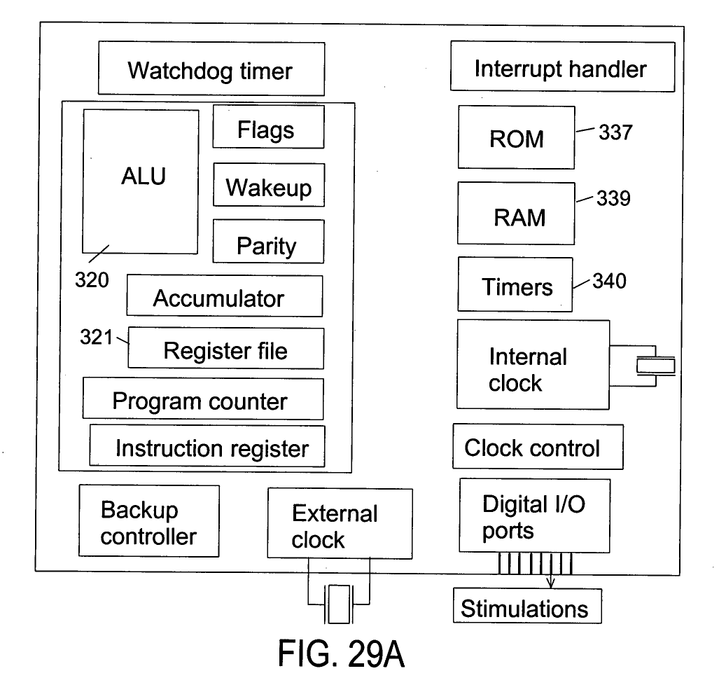

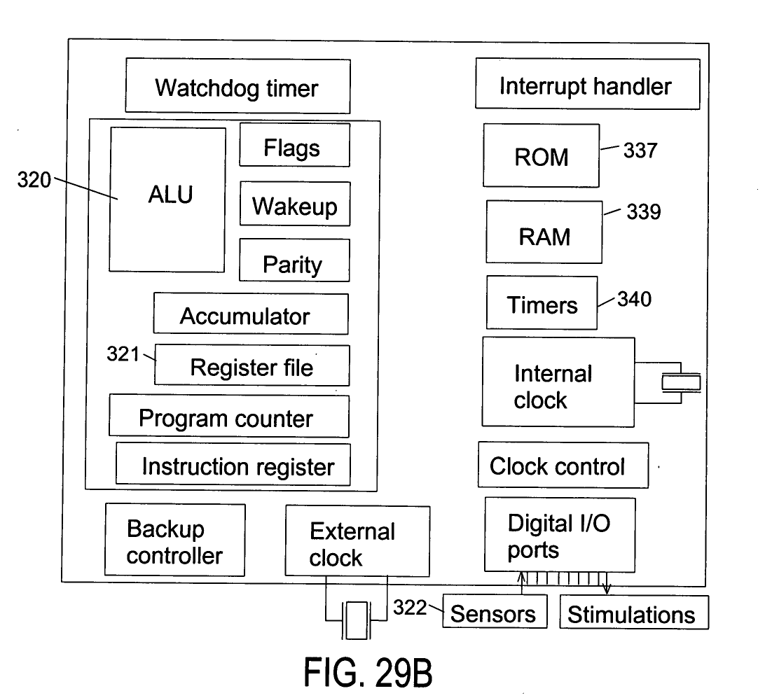

For further details, FIG. 29A highlights the general components of an 8-bit microprocessor as an example. It will be obvious to one skilled in the art that higher level microprocessor, such as a 16-bit or 32-bit may be utilized, and is considered within the scope of this invention. It comprises a ROM 337 to store the instructions of the program to be executed and various programmable parameters, a RAM 339 to store the various intermediate parameters, timers 340 to track the elapsed intervals, a register file 321 to hold intermediate values, an ALU 320 to perform the arithmetic calculation, and other auxiliary units that enhance the performance of a microprocessor-based IPG system.

The size of ROM 337 and RAM 339 units are selected based on the requirements of the algorithms and the parameters to be stored. The number of registers in the register file 321 are decided based upon the complexity of computation and the required number of intermediate values. Timers 340 of different precision are used to measure the elapsed intervals. Even though this embodiment does not have external sensors to control timing, future embodiments may have sensors 322 to effect the timing as shown in conjunction with FIG. 29B.

In this embodiment, the two main components of microprocessor are the datapath and control. The datapath performs the arithmetic operation and the control directs the datapath, memory, and I/O devices to execute the instruction of the program. The hardware components of the microprocessor are designed to execute a set of simple instructions. In general the complexity of the instruction set determines the complexity of datapth elements and controls of the microprocessor.

In this embodiment, the microprocessor is provided with a fixed operating routine. Future embodiments may be provided with the capability of actually introducing program changes in the implanted pulse generator. The instruction set of the microprocessor, the size of the register files, RAM and ROM are selected based on the performance needed and the type of the algorithms used. In this application of pulse generator, in which several algorithms can be loaded and modified, Reduced Instruction Set Computer (RISC) architecture is useful. RISC architecture offers advantages because it can be optimized to reduce the instruction cycle which in turn reduces the run time of the program and hence the current drain. The simple instruction set architecture of RISC and its simple hardware can be used to implement any algorithm without much difficulty. Since size is also a major consideration, an 8-bit microprocessor is used for the purpose, even though other microprocessors may also be used. As most of the arithmetic calculation are based on a few parameters and are rather simple, an accumulator architecture is used to save bits from specifying registers. Each instruction is executed in multiple clock cycles, and the clock cycles are broadly classified into five stages: an instruction fetch, instruction decode, execution, memory reference, and write back stages. Depending on the type of the instruction, all or some of these stages are executed for proper completion.

Initially, an optimal instruction set architecture is selected based on the algorithm to be implemented and also taking into consideration the special needs of a microprocessor based implanted pulse generator (IPG). The instructions are broadly classified into Load/store instructions, Arithmetic and logic instructions (ALU), control instructions and special purpose instructions.

The instruction format is decided based upon the total number of instructions in the instruction set. The instructions fetched from memory are 8 bits long in this example. Each instruction has an opcode field (2 bits), a register specifier field (3-bits), and a 3-bit immediate field. The opcode field indicates the type of the instruction that was fetched. The register specifier indicates the address of the register in the register file on which the operations are performed. The immediate field is shifted and sign extended to obtain the address of the memory location in load/store instruction. Similarly, in branch and jump instruction, the offset field is used to calculate the address of the memory location the control needs to be transferred to.

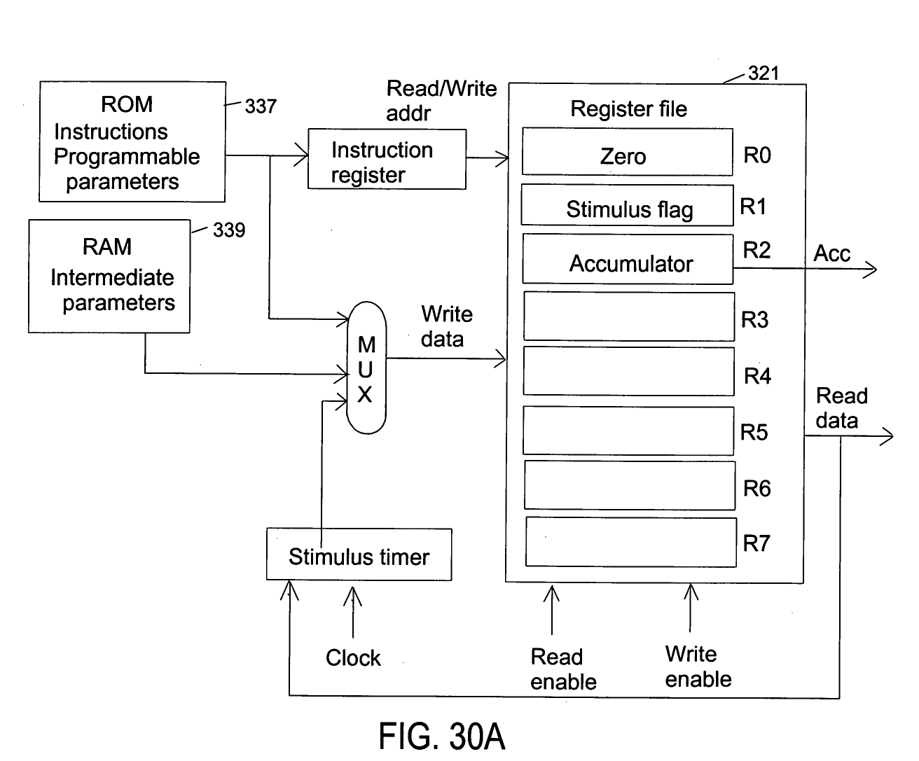

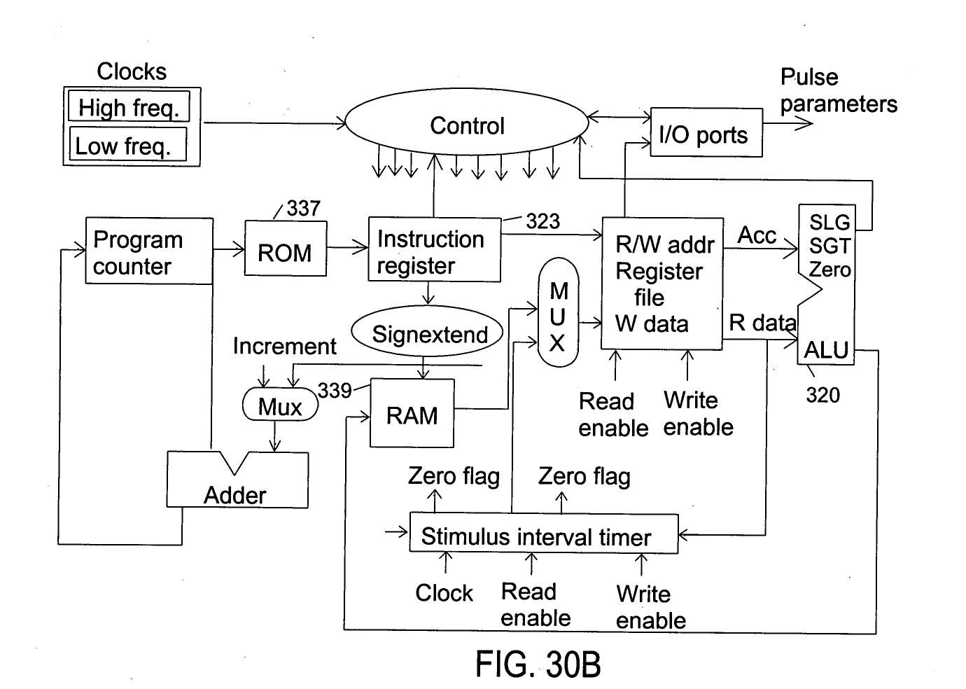

Shown in conjunction with FIG. 30A, the register file 321, which is a collection of registers in which any register can be read from or written to specifying the number of the register in the file. Based on the requirements of the design, the size of the register file is decided. For the purposes of implementation of stimulation pulses algorithms, a register file of eight registers is sufficient, with three special purpose register (0-2) and five general purpose registers (3-7), as shown in FIG. 30A. Register “0” always holds the value “zero”. Register “1” is dedicated to the pulse flags. Register “2” is an accumulator in which all the arithmetic calculations are performed. The read/write address port provides a 3-bit address to identify the register being read or written into. The write data port provides 8-bit data to be written into the registers either from ROM/RAM or timers. Read enable control, when asserted enables the register file to provide data at the read data port. Write enable control enables writing of data being provided at the write data port into a register specified by the read/write address.

Generally, two or more timers are required to implement the algorithm for the IPG. The timers are read and written into just as any other memory location. The timers are provided with read and write enable controls.

The arithmetic logic unit is an important component of the microprocessor. It performs the arithmetic operation such as addition, subtraction and logical operations such as AND and OR. The instruction format of ALU instructions consists of an opcode field (2 bits), a function field (2 bits) to indicate the function that needs to be performed, and a register specifier (3 bits) or an immediate field (4 bits) to provide an operand.

The hardware components discussed above constitute the important components of a datapath. Shown in conjunction with FIG. 30B, there are some special purpose registers such a program counter (PC) to hold the address of the instruction being fetched from ROM 337 and instruction register (IR) 323, to hold the instruction that is fetched for further decoding and execution. The program counter is incremented in each instruction fetch stage to fetch sequential instruction from memory. In the case of a branch or jump instruction, the PC multiplexer allows to choose from the incremented PC value or the branch or jump address calculated. The opcode of the instruction fetched (IR) is provided to the control unit to generate the appropriate sequence of control signals, enabling data flow through the datapath. The register specification field of the instruction is given as read/write address to the register file, which provides data from the specified field on the read data port. One port of the ALU is always provided with the contents of the accumulator and the other with the read data port. This design is therefore referred to as accumulator-based architecture. The sign-extended offset is used for address calculation in branch and jump instructions. The timers are used to measure the elapsed interval and are enabled to count down on a low-frequency clock. The timers are read and written into, just as any other memory location (FIG. 30B).

In a multicycle implementation, each stage of instruction execution takes one clock cycle. Since the datapath takes multiple clock cycles per instruction, the control must specify the signals to be asserted in each stage and also the next step in the sequence. This can be easily implemented as a finite state machine.

A finite state machine consists of a set of states and directions on how to change states. The directions are defined by a next-state function, which maps the current state and the inputs to a new state. Each stage also indicates the control signals that need to be asserted. Every state in the finite state machine takes one clock cycle. Since the instruction fetch and decode stages are common to all the instruction, the initial two states are common to all the instruction. After the execution of the last step, the finite state machine returns to the fetch state.

A finite state machine can be implemented with a register that holds the current stage and a block of combinational logic such as a PLA. It determines the datapath signals that need to be asserted as well as the next state. A PLA is described as an array of AND gates followed by an array of OR gates. Since any function can be computed in two levels of logic, the two-level logic of PLA is used for generating control signals.

The occurrence of a wakeup event initiates a stored operating routine corresponding to the event. In the time interval between a completed operating routine and a next wake up event, the internal logic components of the processor are deactivated and no energy is being expended in performing an operating routine.

A further reduction in the average operating current is obtained by providing a plurality of counting rates to minimize the number of state changes during counting cycles. Thus intervals which do not require great precision, may be timed using relatively low counting rates, and intervals requiring relatively high precision, such as stimulating pulse width, may be timed using relatively high counting rates.

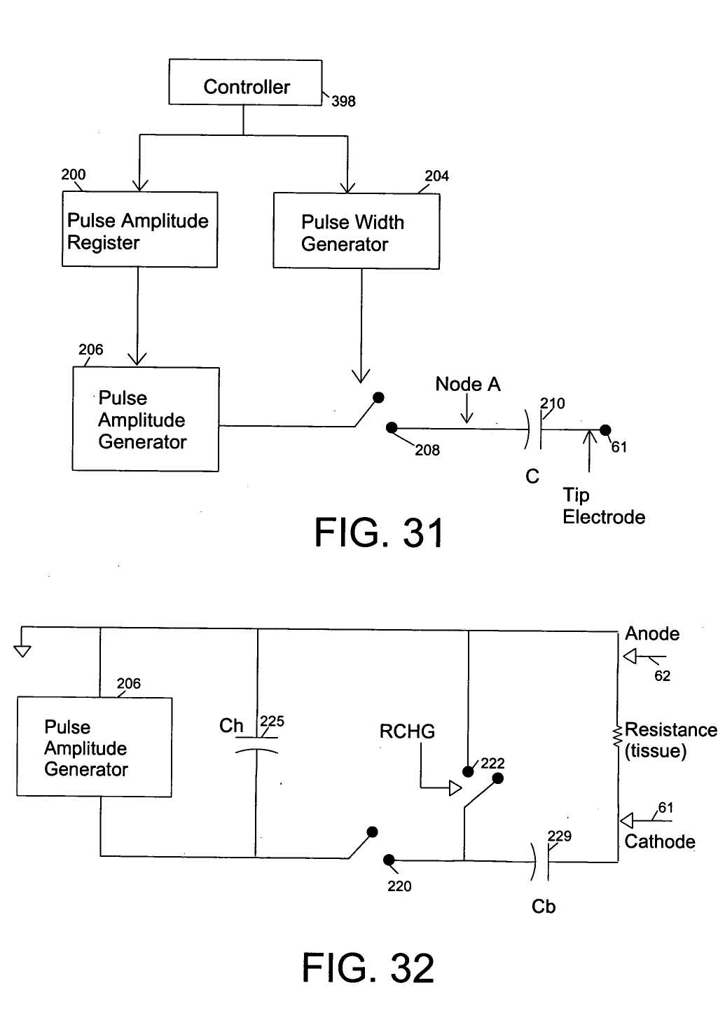

The logic and control unit 398 of the IPG controls the output amplifiers. The pulses have predetermined energy (pulse amplitude and pulse width) and are delivered at a time determined by the therapy stimulus controller. The circuitry in the output amplifier, shown in conjunction with (FIG. 31) generates an analog voltage or current that represents the pulse amplitude. The stimulation controller module initiates a stimulus pulse by closing a switch 208 that transmits the analog voltage or current pulse to the nerve tissue through the tip electrode 61 of the lead 40. The output circuit receiving instructions from the stimulus therapy controller 398 that regulates the timing of stimulus pulses and the amplitude and duration (pulse width) of the stimulus. The pulse amplitude generator 206 determines the configuration of charging and output capacitors necessary to generate the programmed stimulus amplitude. The output switch 208 is closed for a period of time that is controlled by the pulse width generator 204. When the output switch 208 is closed, a stimulus is delivered to the tip electrode 61 of the lead 40.

The constant-voltage output amplifier applies a voltage pulse to the distal electrode (cathode) 61 of the lead 40. A typical circuit diagram of a voltage output circuit is shown in FIG. 32. This configuration contains a stimulus amplitude generator 206 for generating an analog voltage. The analog voltage represents the stimulus amplitude and is stored on a holding capacitor Cₕ 225. Two switches are used to deliver the stimulus pulses to the lead 40, a stimulating delivery. switch 220, and a recharge switch 222, that reestablishes the charge equilibrium after the stimulating pulse has been delivered to the nerve tissue. Since these switches have leakage currents that can cause direct current (DC) to flow into the lead system 40, a DC blocking capacitor C_{b} 229, is included. This is to prevent any possible corrosion that may result from the leakage of current in the lead 40. When the stimulus delivery switch 220 is closed, the pulse amplitude analog voltage stored in the (Cₕ 225) holding capacitor is transferred to the cathode electrode 61 of the lead 40 through the coupling capacitor, C_{b} 229. At the end of the stimulus pulse, the stimulus delivery switch 220 opens. The pulse duration being the interval from the closing of the switch 220 to its reopening. During the stimulus delivery, some of the charge stored on Cₕ 225 has been transferred to C_{b} 229, and some has been delivered to the lead system 40 to stimulate the nerve tissue.

To re-establish equilibrium, the recharge switch 222 is closed, and a rapid recharge pulse is delivered. This is intended to remove any residual charge remaining on the coupling capacitor C_{b} 229, and the stimulus electrodes on the lead (polarization). Thus, the stimulus is delivered as the result of closing and opening of the stimulus delivery 220 switch and the closing and opening of the RCHG switch 222. At this point, the charge on the holding Cₕ 225 must be replenished by the stimulus amplitude generator 206 before another stimulus pulse can be delivered.

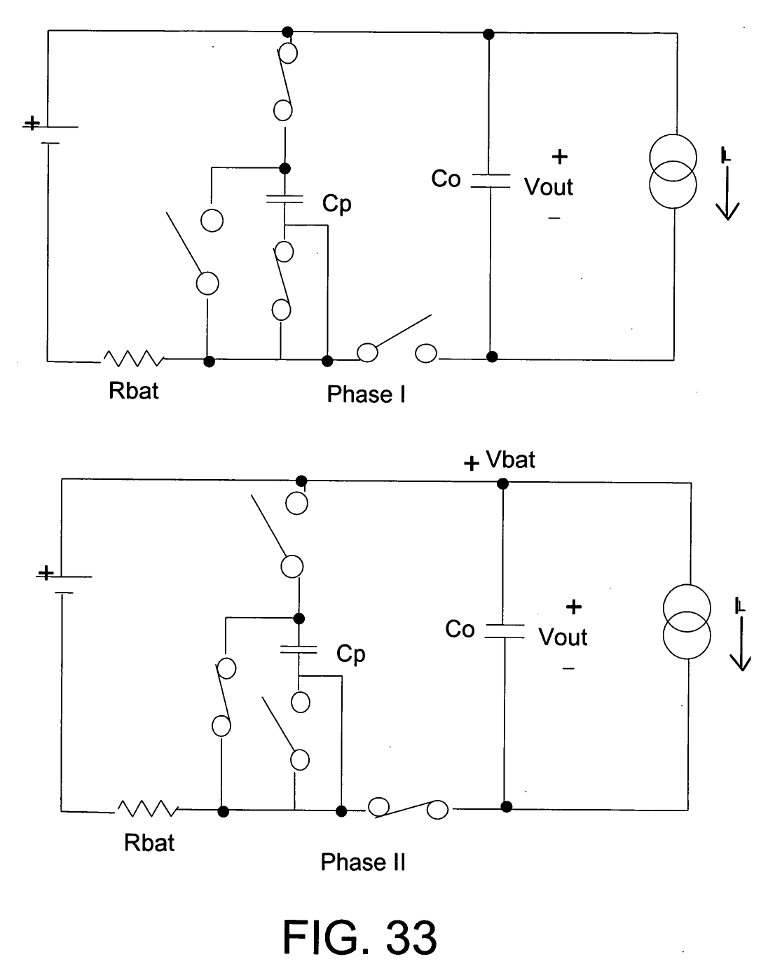

The pulse generating unit charges up a capacitor and the capacitor is discharged when the control (timing) circuitry requires the delivery of a pulse. This embodiment utilizes a constant voltage pulse generator, even though a constant current pulse generator can also be utilized. Pump-up capacitors are used to deliver pulses of larger magnitude than the potential of the batteries. The pump-up capacitors are charged in parallel and discharged into the output capacitor in series. Shown in conjunction with FIG. 33 is a circuit diagram of a voltage doubler which is shown here as an example. For higher multiples of battery voltage, this doubling circuit can be cascaded with other doubling circuits. As shown in FIG. 33, during phase I (top of FIG. 33), the pump capacitor Cₚ is charged to V_{bat} and the output capacitor Cₒ supplies charge to the load. During phase II, the pump capacitor charges the output capacitor, which is still supplying the load current. In this case, the voltage drop across the output capacitor is twice the battery voltage.

FIG. 34A shows one example of the pulse trains that may be delivered with this embodiment or in prior art occipital nerves stimulators. The microcontroller is configured to deliver the pulse train as shown in the figure, i.e. there is “ramping up” of the pulse train. The purpose of the ramping-up is to avoid sudden changes in stimulation, when the pulse train begins. The ramping-up or ramping-down is optional, and may be programmed into the microcontroller.

The prior art systems delivering fixed rectangular pulses provide limited capability for selective stimulation or neuromodulation of occipital nerves. A fixed rectangular pulse, whether constant voltage or constant current, will recruit either i) A-fibers, or ii) A and B fibers, or iii) A and B and C fibers. Only one of these three discrete states can be achieved. This form of modulation is severely limited for providing therapy for neurological disorders.

In the method and system of the current invention, the microcontroller is configured to deliver rectangular, non-rectangular, biphasic, multi-step, and other complex pulses where the amplitude is varying during the pulse. Advantageously, these complex pulses provide a new dimention to selective stimulation or neuromodulation of occipital nerves to provide therapy for chronic headache, transformed migraine, and occipital neuralgia.

Examples of these pulses and pulse trains are shown in FIGS. 34B to 34N. Selective stimulation with these complex pulses takes into account the threshold properties of different types of nerve fibers, as well as, the different refractory properties of different types of nerve fibers that are contained in the occipital nerves For example in the multi-step pulse shown in FIG. 34C, the first part of the pulse will tend to recruit large diameter (and myelinated) fibers, such as A and B fibers. The middle portion of the pulse where the amplitude is highest, will tend to recruit C-fibers which are the smallest fibers, and the last portion of the pulse will again tend to recruit the large diameter fibers provided they are not refractory. The multi-step (and multi-amplitude) pulses shown in FIG. 34E will tend to recruit large diameter fibers initially, and the later part of the pulse will tend to recruit the smaller diameter C-fibers.

Further, as shown in the examples of FIGS. 34F and 34H, complex and simple pulses, or pulse trains may be alternated. The pulses and pulse trains of this disclosure give physicians a lot of flexibility for trying various different neuromodulation algorithms, for providing and optimizing therapy for chronic headache, transformed migraine, and occipital neuralgia.

In one embodiment, tripolar electrodes (not shown) may also be used. The different pulses used in conjunction with tripolar electrodes are shown in conjunction with FIGS. 34-I, 34J, 34K, 34L, 34M, and 34-N. This combination is advantageous, because it can be used to provide selective fiber block as well. The combination of tripolar electrodes and the pulse shapes of FIGS. 34-I to 34-N also reduce the electrical charge of the pulse.

With tripolar electrodes, the electrode consists of a cathode, flanked by two anodes. When stimulation is applied, the nerve membrane is depolarized near the cathode and hyperpolarized near the anodes. If the membrane is sufficiently hyperpolarized, an action potential (AP) that travels into the depolarized zone cannot pass the hyperpolarized zone and is arrested. As with excitation, a lower external stimulus is needed for blocking large diameter fibers than for blocking smaller ones (C-fibers).

As shown in FIGS. 34-I and 34J, the microcontroller 398 in the pulse generator 391 is configured to provide stepped pulses. The current of the first step is too low to induce an action potential (AP), but only depolarizes the membrane. The AP is generated during the second step. The pulses in FIG. 34-I and 34J are similar, except that the pulses in FIG. 34-I have a longer first step. In addition to anodel blocking, another advantage of these stepped pulses is that the total charge per pulse can be reduced by almost a third.

Other examples of complex pulses, that may be used with tripolar electrodes are shown in FIGS. 34-I to 34-N. FIG. 34K shows biphasic pulses with a time delay t_{d} between the positive and negative pulse. FIG. 34L shows biphasic pulses with a time delay t_{d}, where the second part of the pulse is a step pulse. FIG. 34M shows ramp pulses, and FIG. 34-N show pulses with exponential components. Theoretical work, computer modeling, and animal studies have all shown that lower charge is obtained with these modified pulses when compared to square pulses. The charge reduction of these pulses can be approximately 30% less when compared to square pulses, which is fairly significant. The microcontroller 398 of the pulse generator 391 can be configured to deliver these pulses, as is well known to one skilled in the art.

Since the number of types of pulses and pulse trains to provide therapy can be complex for many physician's, in one aspect pre-determined/pre-packaged program comprise a complete program for the pulse trains that deliver therapy. The advantage of the pre-packaged programs is that the physician may program a complicated program simply by selecting a program number.

### Programming

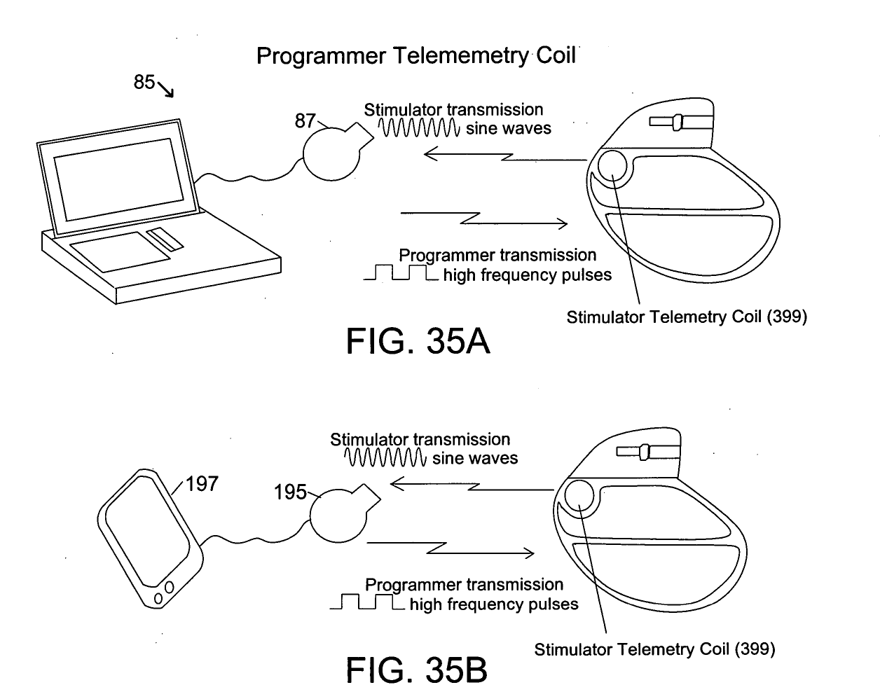

The programming of the implanted pulse generator (IPG) 391 is shown in conjunction with FIGS. 35A and 35B. With the magnetic Reed Switch 389 (FIG. 26) in the closed position, a coil in the head of the programmer 85, communicates with a telemetry coil 399 of the implanted pulse generator 391. Bi-directional inductive telemetry is used to exchange data with the implanted unit 391 by means of the external programming unit 85.

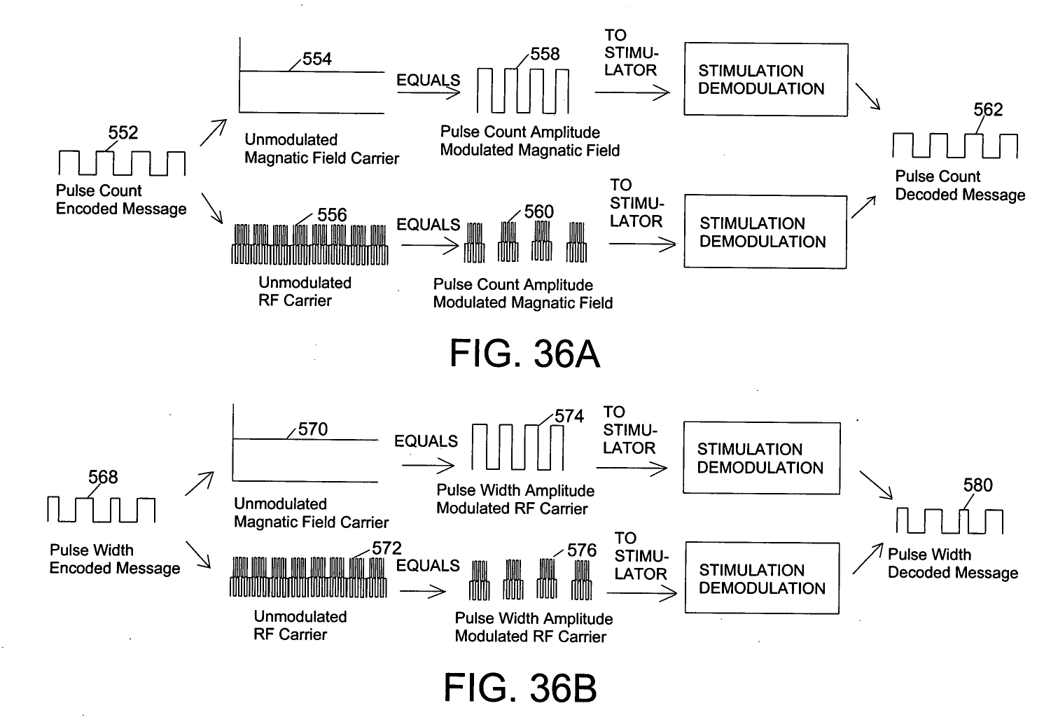

The transmission of programming information involves manipulation of the carrier signal in a manner that is recognizable by the pulse generator 391 as a valid set of instructions. The process of modulation serves as a means of encoding the programming instruction in a language that is interpretable by the implanted pulse generator 391. Modulation of signal amplitude, pulse width, and time between pulses are all used in the programming system, as will be appreciated by those skilled in the art. FIG. 36A shows an example of pulse count modulation, and FIG. 36B shows an example of pulse width modulation, that can be used for encoding.

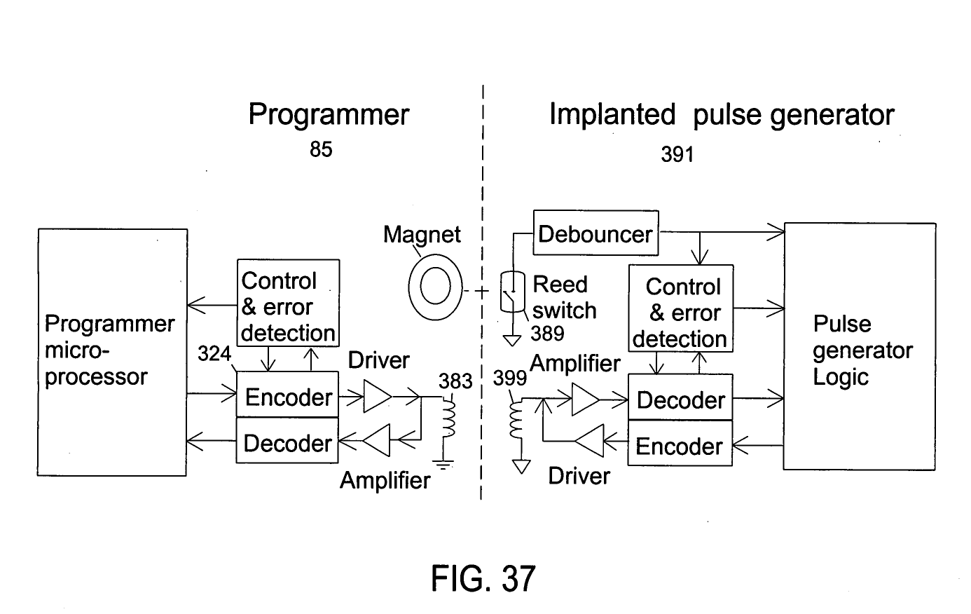

FIG. 37 shows a simplified overall block diagram of the implanted pulse generator (IPG) 391 programming and telemetry interface. The left half of FIG. 37 is programmer 85 which communicates programming and telemetry information with the IPG 391. The sections of the IPG 391 associated with programming and telemetry are shown on the right half of FIG. 37. In this case, the programming sequence is initiated by bringing a permanent magnet in the proximity of the IPG 391 which closes a reed switch 389 in the IPG 391. Information is then encoded into a special error-correcting pulse sequence and transmitted electromagnetically through a set of coils. The received message is decoded, checked for errors, and passed on to the unit's logic circuitry. The IPG 391 of this embodiment includes the capability of bi-directional communication.

The reed switch 389 is a magnetically-sensitive mechanical switch, which consists of two thin strips of metal (the “reed”) which are ferromagnetic. The reeds normally spring apart when no magnetic field is present. When a field is applied, the reeds come together to form a closed.circuit because doing so creates a path of least reluctance. The programming head of the programmer contains a high-field-strength ceramic magnet.

When the switch closes, it activates the programming hardware, and initiates an interrupt of the IPG central processor. Closing the reed switch 389 also presents the logic used to encode and decode programming and telemetry signals. A nonmaskable interrupt (NMI) is sent to the IPG processor, which then executes special programming software. Since the NMI is an edge-triggered signal and the reed switch is vulnerable to mechanical bounce, a debouncing circuit is used to avoid multiple interrupts. The overall current consumption of the IPG increases during programming because of the debouncing circuit and other communication circuits.

A coil 399 is used as an antenna for both reception and transmission . Another set of coils 383 is placed in the programming head, a relatively small sized unit connected to the programmer 85. All coils are tuned to the same resonant frequency. The interface is half-duplex with one unit transmitting at a time.

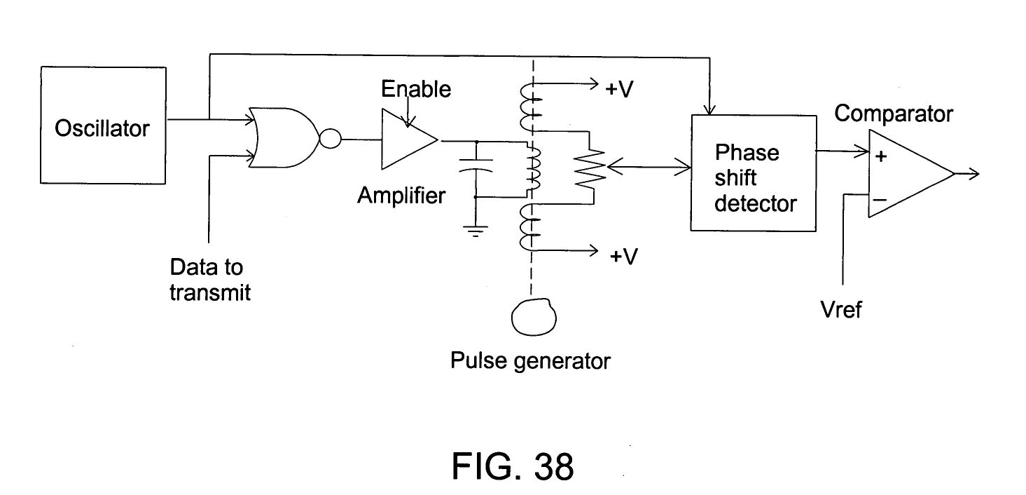

Since the relative positions of the programming head 87and IPG 391 determine the coupling of the coils, this embodiment utilizes a special circuit which has been devised to aid the positioning of the programming head, and is shown in FIG. 38. It operates on similar principles to the linear variable differential transformer. An oscillator tuned to the resonant frequency of the pacemaker coil 399 drives the center coil of a three-coil set in the programmer head. The phase difference between the original oscillator signal and the resulting signal from the two outer coils is measured using a phase shift detector. It is proportional to the distance between the implanted pulse generator and the programmer head. The phase shift, as a voltage, is compared to a reference voltage and is then used to control an indicator such as an LED. An enable signal allows switching the circuit on and off.

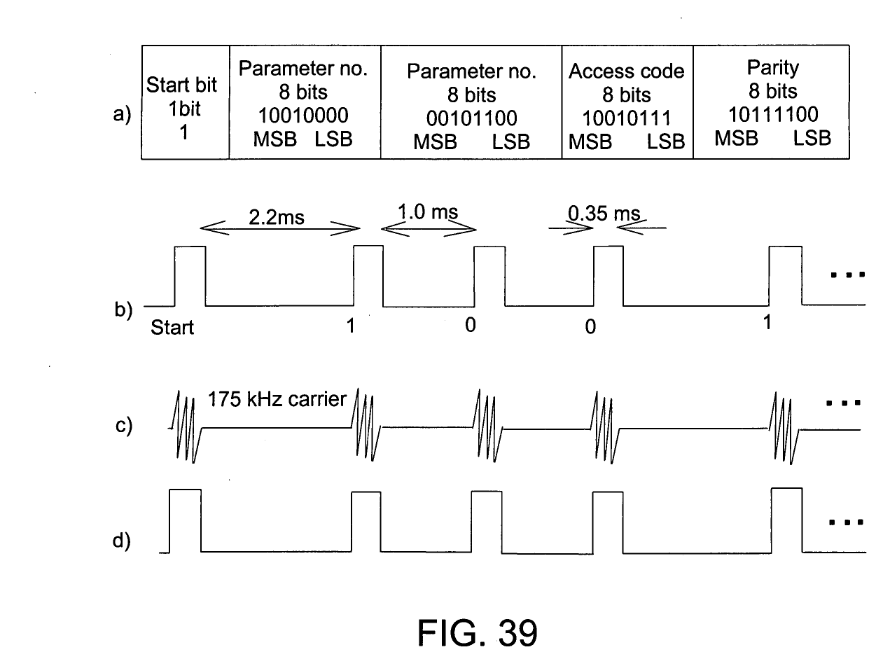

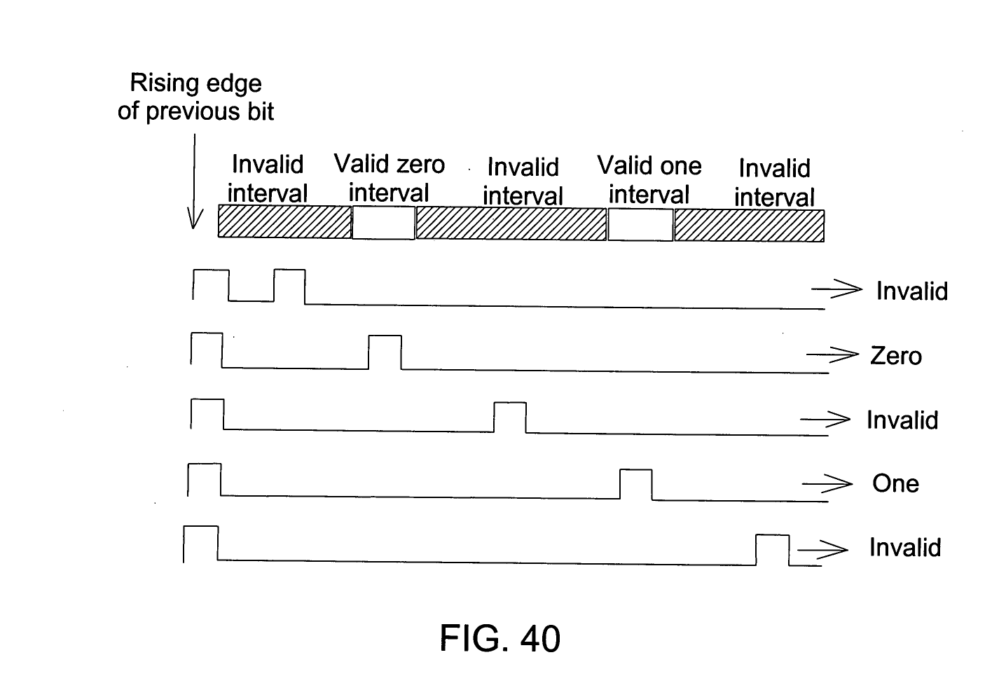

Actual programming is shown in conjunction with FIGS. 39 and 40. Programming and telemetry messages comprise many bits; however, the coil interface can only transmit one bit at a time. In addition, the signal is modulated to the resonant frequency of the coils, and must be transmitted in a relatively short period of time, and must provide detection of erroneous data.

A programming message is comprised of five parts FIG. 39(a). The start bit indicates the beginning of the message and is used to synchronize the timing of the rest of the message. The parameter number specifies which parameter (e.g., mode, pulse width, delay) is to be programmed. In the example, in FIG. 33(a) the number 10010000 specifies the pulse rate to be specified. The parameter value represents the value that the parameter should be set to. This value may be an index into a table of possible values; for example, the value 00101100 represents a pulse stimulus rate of 80 pulses/min. The access code is a fixed number based on the stimulus generator model which must be matched exactly for the message to succeed. It acts as a security mechanism against use of the wrong programmer, errors in the message, or spurious programming from environmental noise. It can also potentially allow more than one programmable implant in the patient. Finally, the parity field is the bitwise exclusive-OR of the parameter number and value fields. It is one of several error-detection mechanisms.

All of the bits are then encoded as a sequence of pulses of 0.35-ms duration FIG. 39(b). The start bit is a single pulse. The remaining bits are delayed from their previous bit according to their bit value. If the bit is a zero, the delay is short (1.0); if it is a one, the delay is long (2.2 ms). This technique of pulse position coding, makes detection of errors easier.

The serial pulse sequence is then amplitude modulated for transmission FIG. 39(c). The carrier frequency is the resonant frequency of the coils. This signal is transmitted from one set of coils to the other and then demodulated back into a pulse sequence FIG. 39(d).

FIG. 40 shows how each bit of the pulse sequence is decoded from the demodulated signal. As soon as each bit is received, a timer begins timing the delay to the next pulse. If the pulse occurs within a specific early interval, it is counted as a zero bit (FIG. 40(b)). If it otherwise occurs with a later interval, it is considered to be a one bit (FIG. 40(d)). Pulses that come too early, too late, or between the two intervals are considered to be errors and the entire message is discarded (FIG.40 (a, c, e)). Each bit begins the timing of the bit that follows it. The start bit is used only to time the first bit.

Telemetry data may be either analog or digital. Digital signals are first converted into a serial bit stream using an encoding such as shown in FIG. 40 (b). The serial stream or the analog data is then frequency modulated for transmission.

An advantage of this and other encodings is that they provide multiple forms of error detection. The coils and receiver circuitry are tuned to the modulation frequency, eliminating noise at other frequencies. Pulse-position coding can detect errors by accepting pulses only within narrowly-intervals. The access code acts as a security key to prevent programming by spurious noise or other equipment. Finally, the parity field and other checksums provides a final verification that the message is valid. At any time, if an error is detected, the entire message is discarded.

Another more sophisticated type of pulse position modulation may be used to increase the bit transmission rate. In this, the position of a pulse within a frame is encoded into one of a finite number of values, e.g. 16. A special synchronizing bit is transmitted to signal the start of the frame. Typically, the frame contains a code which specifies the type or data contained in the remainder of the frame.

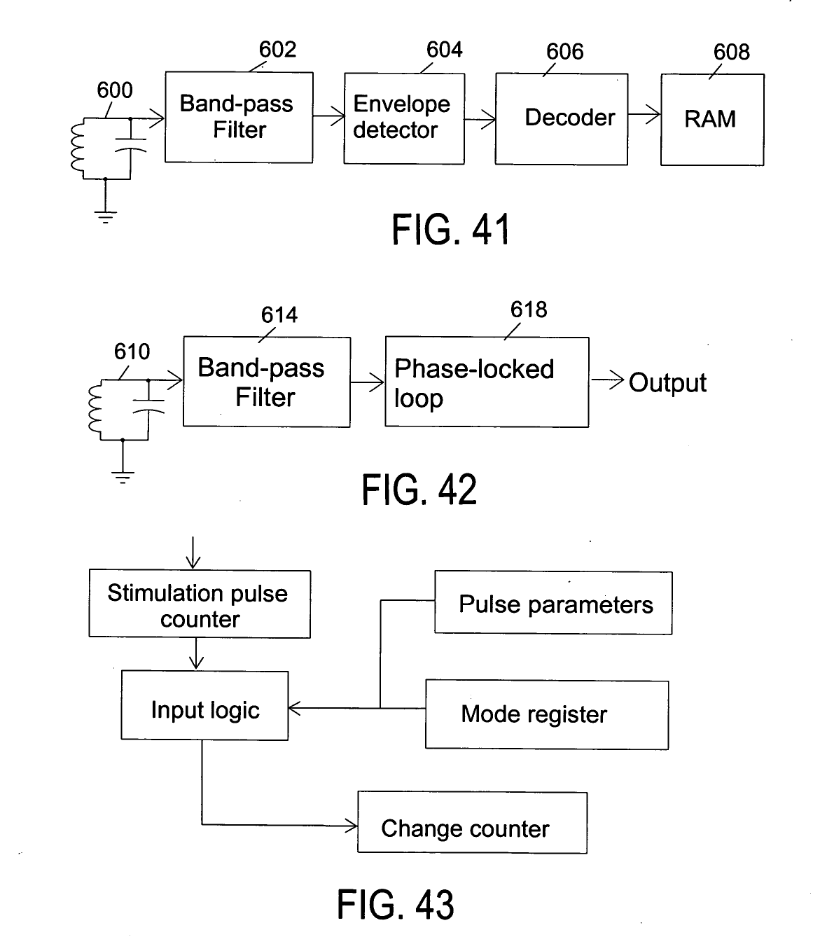

FIG. 41 shows a diagram of receiving and decoding circuitry for programming data. The IPG coil, in parallel with capacitor creates a tuned circuit for receiving data. The signal is band-pass filtered 602 and envelope detected 604 to create the pulsed signal in FIG. 39 (d). After decoding, the parameter value is placed in a RAM at the location specified by the parameter number. The IPG can have two copies of the RAM—a permanent set and a temporary set—which makes it easy for the physician to set the IPG to a temporary configuration and later reprogram it back to the usual settings.

FIG. 42 shows the basic circuit used to receive telemetry data. Again, a coil and capacitor create a resonant circuit tuned to the carrier frequency. The signal is further band-pass filtered 614 and then frequency-demodulated using a phase-locked loop 618.

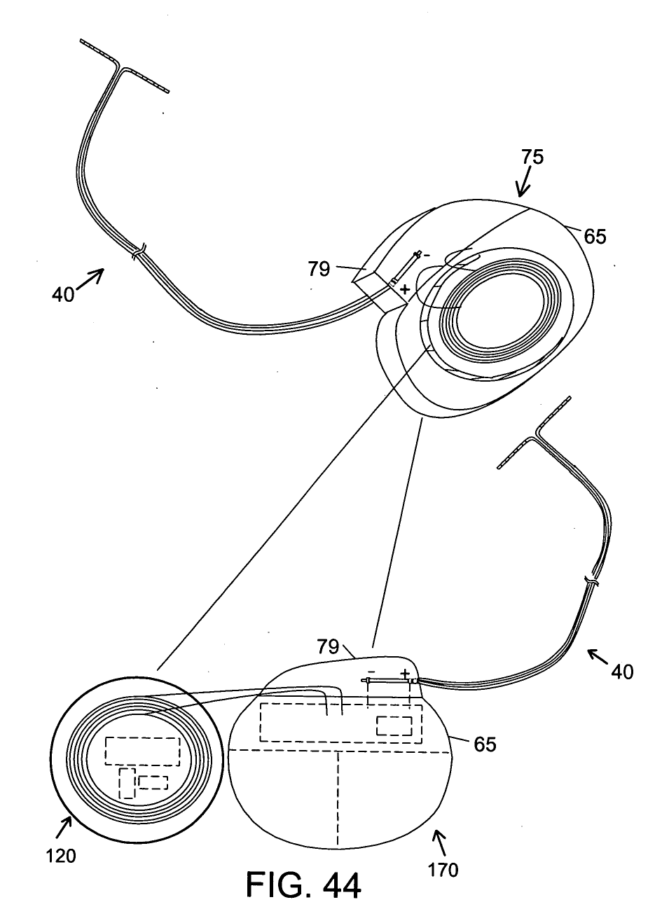

This embodiment also comprises an optional battery status test circuit. Shown in conjunction with FIG. 43, the charge delivered by the battery is estimated by keeping track of the number of pulses delivered by the IPG 391. An internal charge counter is updated during each test mode to read the total charge delivered. This information about battery status is read from the IPG 391 via telemetry.

### Combination Implantable Device Comprising Both A Stimulus-Receiver And A Programmable Implantable Pulse Generator (IPG)

In one embodiment, the implantable device may comprise both a stimulus-receiver and a programmable implantable pulse generator (IPG) in one device. Another embodiment of a similar device is disclosed in applicant's co-pending application Ser. No. 10/436,017. This embodiment also comprises predetermined/pre-packaged programs. Examples of several stimulation states were given in the previous sections, under “Programmer-less Implantable Pulse Generator (IPG)” and “Programmable Implantable Pulse Generator”. These predetermined/pre-packaged programs comprise unique combinations of pulse amplitude, pulse width, pulse frequency, ON-time and OFF-time.

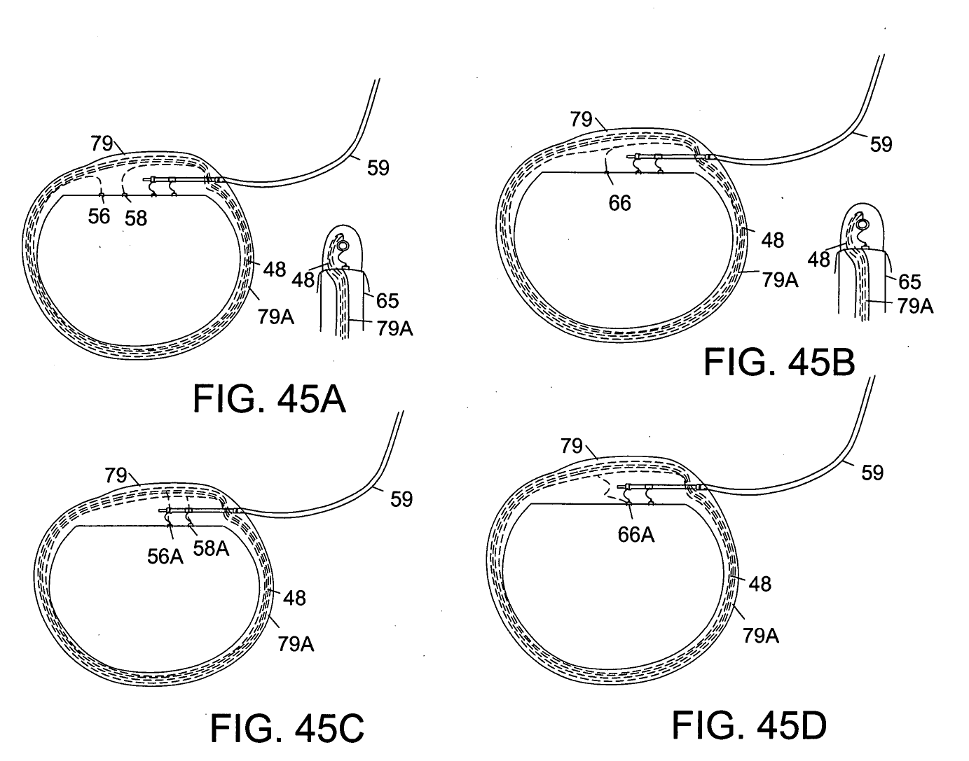

FIG. 44 shows a close up view of the packaging of the implanted stimulator 75 of this embodiment, showing the two subassemblies 120, 170. The two subassemblies are the stimulus-receiver module 120 and the battery operated pulse generator module 170. The electrical components of the stimulus-receiver module 120 may be substantially in the titanium case along with other circuitry, except for a coil. The coil may be outside the titanium case as shown in FIG. 44, or the coil 48C may be externalized at the header portion 79 of the implanted device, and may be wrapped around the titanium can. In this case, the coil is encased in the same material as the header 79, as shown in FIGS. 45A-45D. FIG. 45A depicts a bipolar configuration with two separate feed-throughs, 56, 58. FIG. 45B depicts a unipolar configuration with one separate feed-through 66. FIG. 45C, and 45D depict the same configuration except the feed-throughs are common with the feed-throughs 66A for the lead.

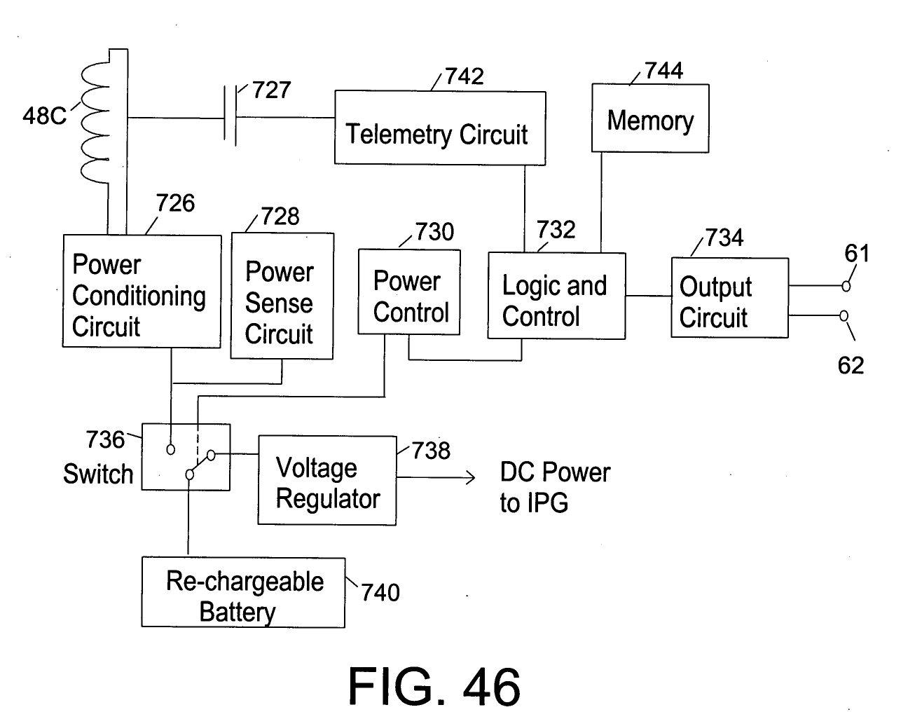

FIG. 46 is a simplified overall block diagram of the embodiment where the implanted stimulator 75 is a combination device, which may be used as a stimulus-receiver (SR) in conjunction with an external stimulator, or the same implanted device may be used as a traditional programmable implanted pulse generator (IPG). The coil 48C which is external to the titanium case may be used both as a secondary of a stimulus-receiver, or may also be used as the forward and back telemetry coil.

In this embodiment, as disclosed in FIG. 46, the IPG circuitry within the titanium case is used for all stimulation pulses whether the energy source is the internal battery 740 or an external power source. The external device serves as a source of energy, and as a programmer that sends telemetry to the IPG. For programming, the energy is sent as high frequency sine waves with superimposed telemetry wave driving the external coil 46C. Once received by the implanted coil 48C, the telemetry is passed through coupling capacitor 727 to the IPG's telemetry circuit 742. For pulse delivery using external power source, the stimulus-receiver portion will receive the energy coupled to the implanted coil 48C and, using the power conditioning circuit 726, rectify it to produce DC, filter and regulate the DC, and couple it to the IPG's voltage regulator 738 section so that the IPG can run from the externally supplied energy rather than the implanted battery 740.

The system provides a power sense circuit 728 that senses the presence of external power communicated with the power control 730 when adequate and stable power is available from an external source. The power control circuit controls a switch 736 that selects either battery power 740 or conditioned external power from 726. The logic and control section 732 and memory 744 includes the IPG's microcontroller, pre-programmed instructions, and stored changeable parameters. Using input for the telemetry circuit 742 and power control 730, this section controls the output circuit 734 that generates the output pulses.

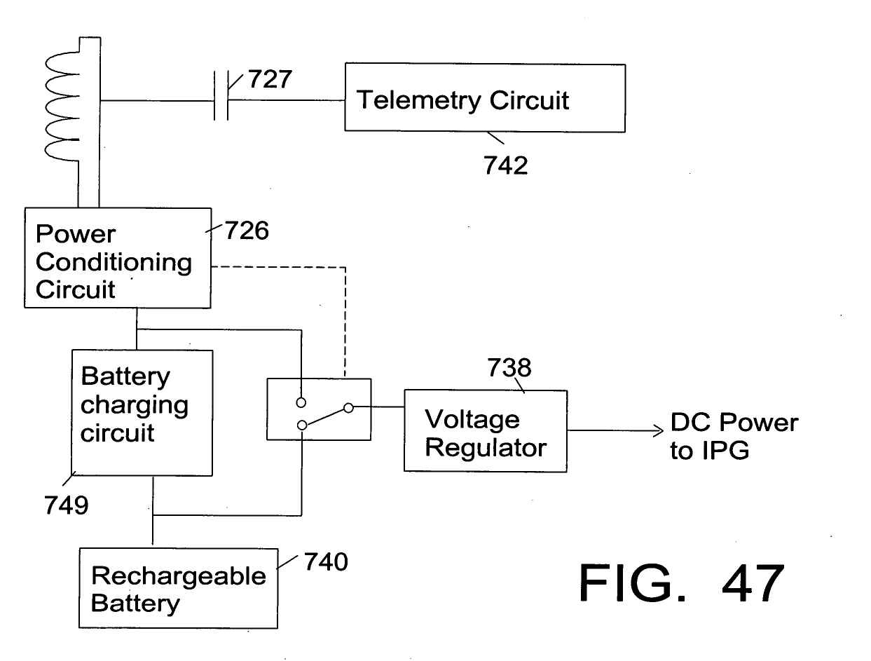

It will be clear to one skilled in the art that this embodiment of the invention can also be practiced with a rechargeable battery. This version is shown in conjunction with FIG. 47. The circuitry in the two versions are similar except for the battery charging circuitry 749. This circuit is energized when external power is available. It senses the charge state of the battery and provides appropriate charge current to safely recharge the battery without overcharging.

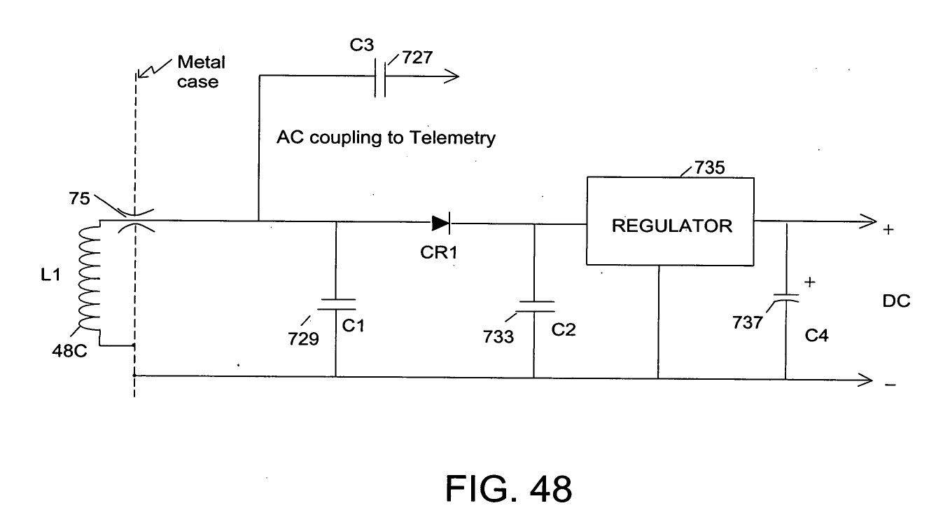

The stimulus-receiver portion of the circuitry is shown in conjunction with FIG. 48. Capacitor C1 (729) makes the combination of C1 and L1 sensitive to the resonant frequency and less sensitive to other frequencies, and energy from an external (primary) coil 46C is inductively transferred to the implanted unit via the secondary coil 48C. The AC signal is rectified to DC via diode 731, and filtered via capacitor 733. A regulator 735 sets the output voltage and limits it to a value just above the maximum IPG cell voltage. The output capacitor C4 (737), typically a tantalum capacitor with a value of 100 micro-Farads or greater, stores charge so that the circuit can supply the IPG with high values of current for a short time duration with minimal voltage change during a pulse while the current draw from the external source remains relatively constant. Also shown in conjunction with FIG. 48, a capacitor C3 (727) couples signals for forward and back telemetry.

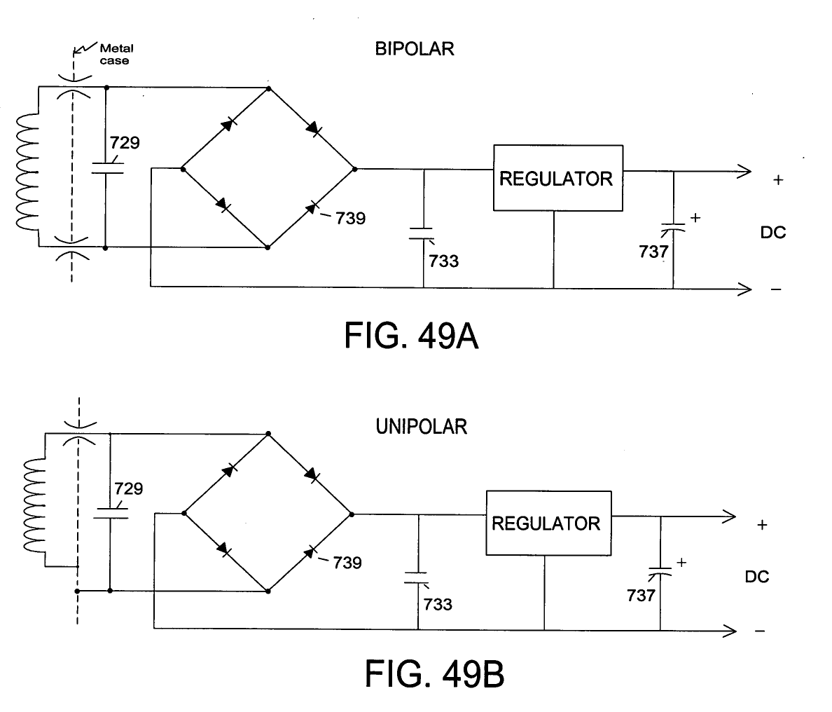

FIGS. 49A and 49B show alternate connection of the receiving coil. In FIG. 49A, each end of the coil is connected to the circuit through a hermetic feedthrough filter. In this instance, the DC output is floating with respect to the IPG's case. In FIG. 49B, one end of the coil is connected to the exterior of the IPG's case. The circuit is completed by connecting the capacitor 729 and bridge rectifier 739 to the interior of the IPG's case The advantage of this arrangement is that it requires one less hermetic feedthrough filter, thus reducing the cost and improving the reliability of the IPG. Hermetic feedthrough filters are expensive and a possible failure point. However, the case connection may complicit the output circuitry or limit its versatility. When using a bipolar electrode, care must be taken to prevent an unwanted return path for the pulse to the IPG's case. This is not a concern for unipolar pulses using a single conductor electrode because it relies on the IPG's case a return for the pulse current.

In the unipolar configuration, advantageously a bigger tissue area is stimulated since the difference between the tip (cathode) and case (anode) is larger. Stimulation using both configuration is considered within the scope of this invention.

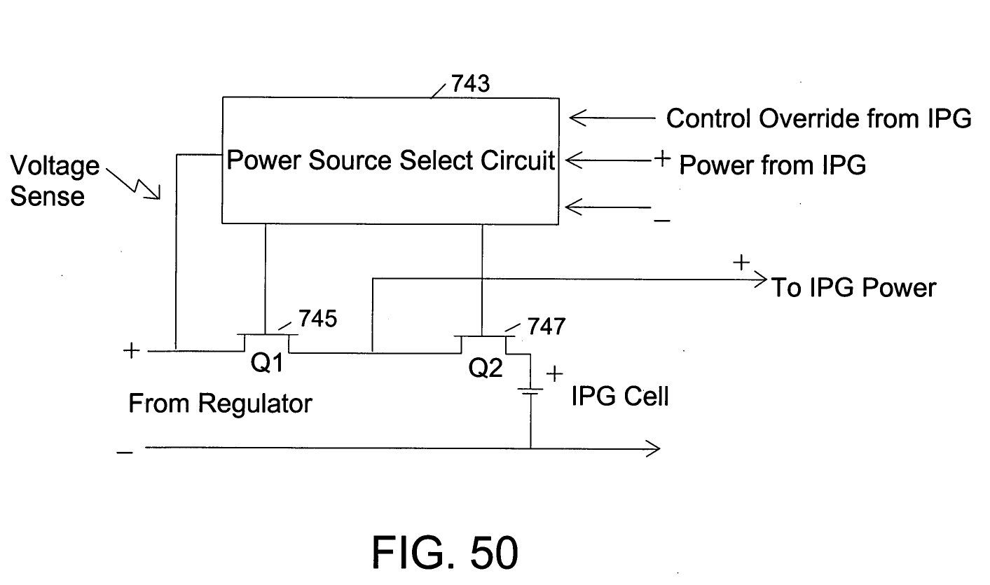

The power source select circuit is highlighted in conjunction with FIG. 50. In this embodiment, the IPG provides stimulation pulses according to the stimulation programs stored in the memory 744 of the implanted stimulator, with power being supplied by the implanted battery 740. When stimulation energy from an external stimulator is inductively received via secondary coil 48C, the power source select circuit (shown in block 743) switches power via transistor Q1 745 and transistor Q2 743. Transistor Q1 and Q2 are preferably low loss MOS transistor used as switches, even though other types of transistors may be used.

### Implantable Pulse Generator (IPG) Comprising A Rechargeable Battery

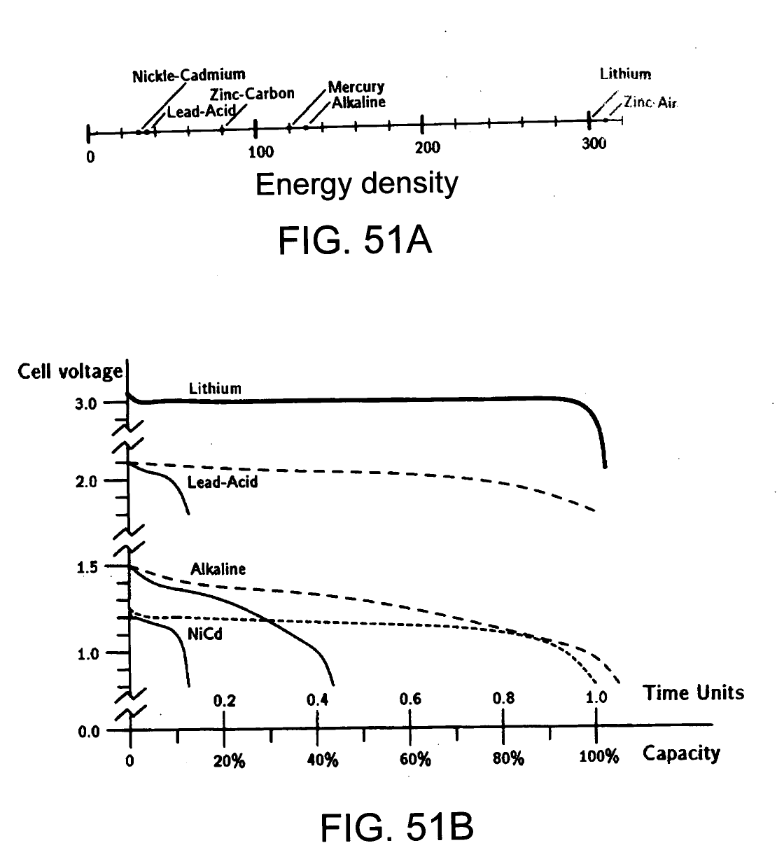

In one embodiment, an implantable pulse generator with rechargeable power source can be used. Because of the rapidity of the pulses required for modulating the occipital nerves with stimulating and/or blocking pulses, there is a real need for power sources that will provide an acceptable service life under conditions of continuous delivery of high frequency pulses. FIG. 51A shows a graph of the energy density of several commonly used battery technologies. Lithium batteries have by far the highest energy density of commonly available batteries. Also, a lithium battery maintains a nearly constant voltage during discharge. This is shown in conjunction with FIG. 51B, which is normalized to the performance of the lithium battery. Lithium-ion batteries also have a long cycle life, and no memory effect. However, Lithium-ion batteries are not as tolerant to overcharging and overdischarging. One of the most recent development in rechargeable battery technology is the Lithium-ion polymer battery. Recently the major battery manufacturers (Sony, Panasonic, Sanyo) have announced plans for Lithium-ion polymer battery production.

This embodiment also comprises predetermined/pre-packaged programs. Examples of several stimulation states were given in the previous sections, under “Programmer-less Implantable Pulse Generator (IPG)” and “Programmable Implantable Pulse Generator”. These pre-packaged/pre-determined programs comprise unique combinations of pulse amplitude, pulse width, pulse frequency, ON-time and OFF-time. Additionally, predetermined programs comprising blocking pulses may also be stored in the memory of the pulse generator.

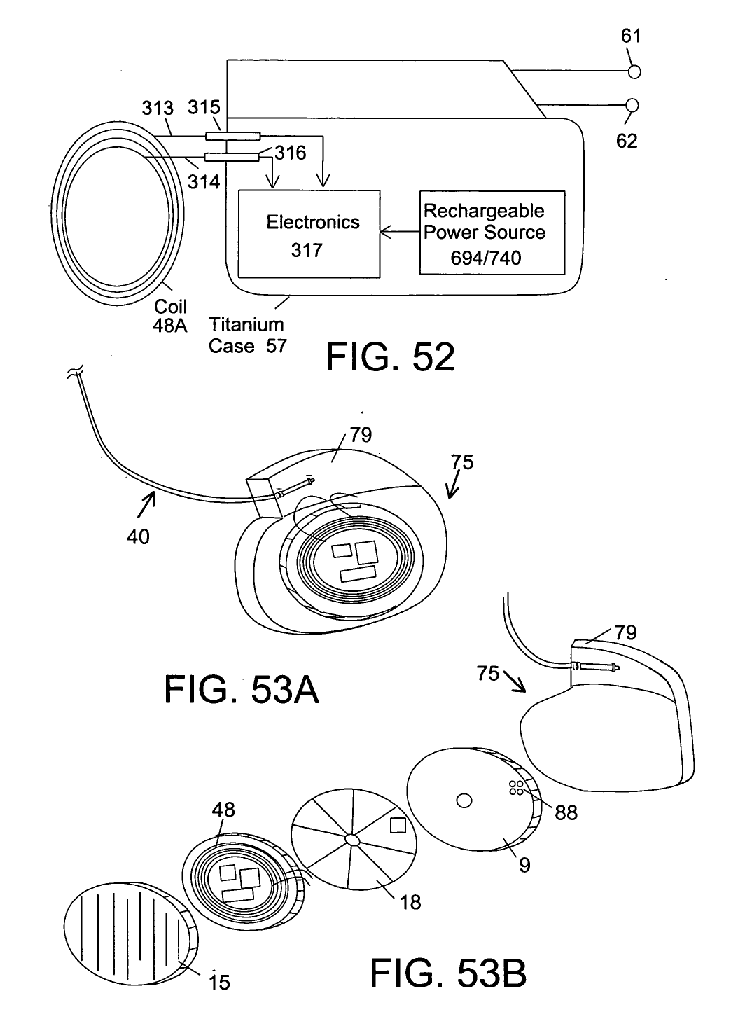

As shown in conjunction with FIG. 52, the coil is externalized from the titanium case 57. The RF pulses transmitted via coil 46 and received via subcutaneous coil 48A are rectified via a diode bridge. These DC pulses are processed and the resulting current applied to recharge the battery 694/740 in the implanted pulse generator. In one embodiment the coil 48C may be externalized at the header portion 79 of the implanted device, and may be wrapped around the titanium can, as was previously shown in FIGS. 45A-D.

In one embodiment, the coil may also be positioned on the titanium case as shown in conjunction with FIGS. 53A and 53B. FIG. 53A shows a diagram of the finished implantable stimulator 391R of one embodiment. FIG. 53B shows the pulse generator with some of the components used in assembly in an exploded view. These components include a coil cover 15, the secondary coil 48 and associated components, a magnetic shield 18, and a coil assembly carrier 19. The coil assembly carrier 9 has at least one positioning detail 88 located between the coil assembly and the feed through for positioning the electrical connection. The positioning detail 13 secures the electrical connection.

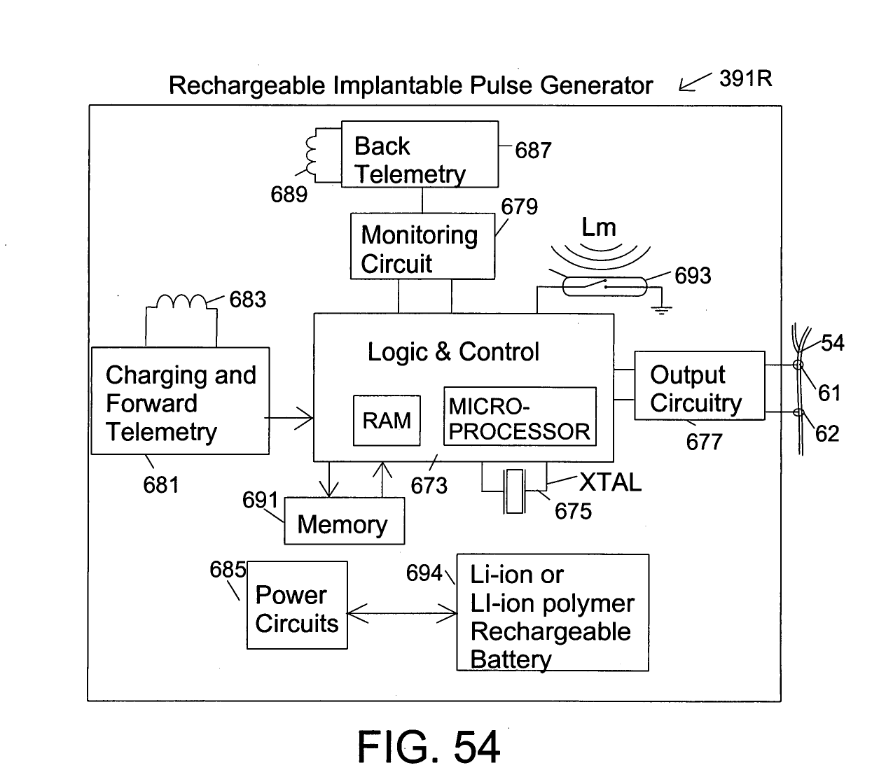

A schematic diagram of the implanted pulse generator (IPG 391R), with re-chargeable battery 694, is shown in conjunction with FIG. 54. The IPG 391R includes logic and control circuitry 673 connected to memory circuitry 691. The operating program and stimulation parameters are typically stored within the memory 691 via forward telemetry. Stimulation pulses are provided to the occipital nerves via output circuitry 677 controlled by the microcontroller.

The operating power for the IPG 391R is derived from a rechargeable power source 694. The rechargeable power source 694 comprises a rechargeable lithium-ion or lithium-ion polymer battery. Recharging occurs inductively from an external charger to an implanted coil 48B underneath the skin 60. The rechargeable battery 694 may be recharged repeatedly as needed. Additionally, the IPG 391R is able to monitor and telemeter the status of its rechargeable battery 691 each time a communication link is established with the external programmer 85.

Much of the circuitry included within the IPG 391R may be realized on a single application specific integrated circuit (ASIC). This allows the overall size of the IPG 391R to be quite small, and readily housed within a suitable hermetically-sealed case. The IPG case is preferably made from a titanium and is shaped in a rounded case.

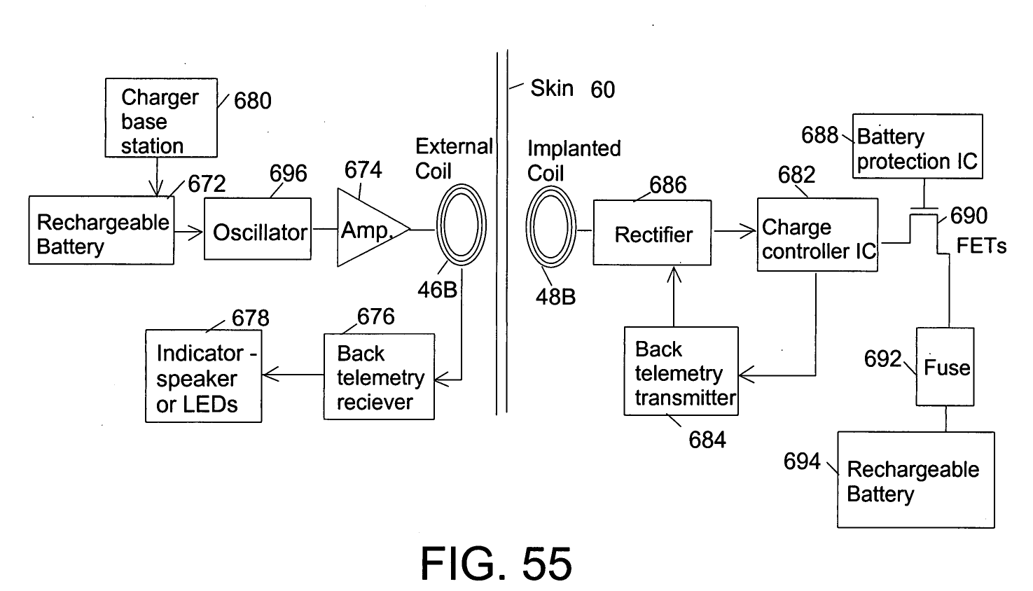

Shown in conjunction with FIG. 55 are the recharging elements of this embodiment. The re-charging system uses a portable external charger to couple energy into the power source of the IPG 391R. The DC-to-AC conversion circuitry 696 of the re-charger receives energy from a battery 672 in the re-charger. A charger base station 680 and conventional AC power line may also be used. The AC signals amplified via power amplifier 674 are inductively coupled between an external coil 46B and an implanted coil 48B located subcutaneously with the implanted pulse generator (IPG) 391R. The AC signal received via implanted coil 48B is rectified 686 to a DC signal which is used for recharging the rechargeable battery 694 of the IPG, through a charge controller IC 682. Additional circuitry within the IPG 391R includes, battery protection IC 688 which controls a FET switch 690 to make sure that the rechargeable battery 694 is charged at the proper rate, and is not overcharged. The battery protection IC 688 can be an off-the-shelf IC available from Motorola (part no. MC 33349N-3R1). This IC monitors the voltage and current of the implanted rechargeable battery 694 to ensure safe operation. If the battery voltage rises above a safe maximum voltage, the battery protection IC 688 opens charge enabling FET switches 690, and prevents further charging. A fuse 692 acts as an additional safeguard, and disconnects the battery 694 if the battery charging current exceeds a safe level. As also shown in FIG. 55, charge completion detection is achieved by a back-telemetry transmitter 684, which modulates the secondary load by changing the full-wave rectifier into a half-wave rectifier/voltage clamp. This modulation is in turn, sensed by the charger as a change in the coil voltage due to the change in the reflected impedance. When detected through a back telemetry receiver 676, either an audible alarm is generated or a LED is turned on.

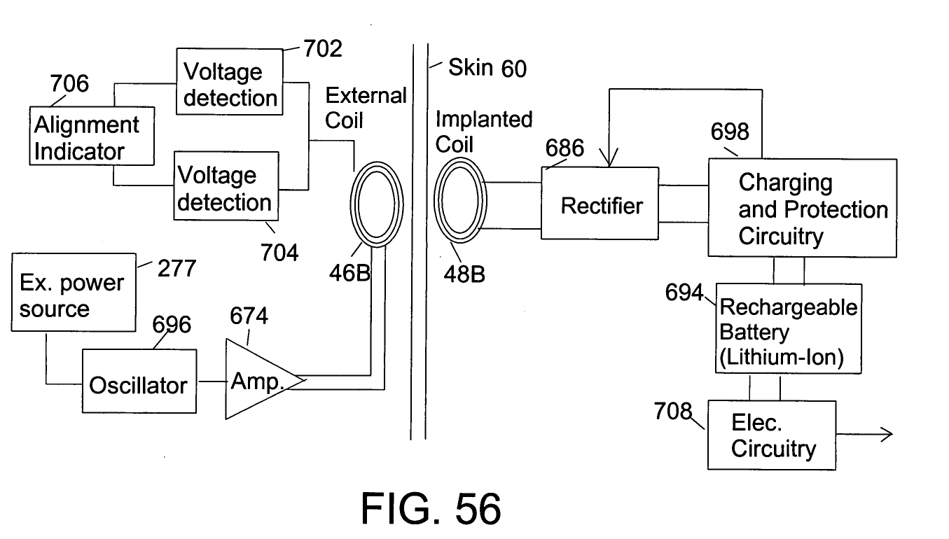

A simplified block diagram of charge completion and misalignment detection circuitry is shown in conjunction with FIG. 56. As shown, a switch regulator 686 operates as either a full-wave rectifier circuit or a half-wave rectifier circuit as controlled by a control signal (CS) generated by charging and protection circuitry 698. The energy induced in implanted coil 48B (from external coil 46B) passes through the switch rectifier 686 and charging and protection circuitry 698 to the implanted rechargeable battery 694. As the implanted battery 694 continues to be charged, the charging and protection circuitry 698 continuously monitors the charge current and battery voltage. When the charge current and battery voltage reach a predetermined level, the charging and protection circuitry 698 triggers a control signal. This control signal causes the switch rectifier 686 to switch to half-wave rectifier operation. When this change happens, the voltage sensed by voltage detector 702 causes the alignment indicator 706 to be activated. This indicator 706 may be an audible sound or a flashing LED type of indicator.

The indicator 706 may similarly be used as a misalignment indicator. In normal operation, when coils 46B (external) and 48B (implanted) are properly aligned, the voltage V_{S} sensed by voltage detector 704 is at a minimum level because maximum energy transfer is taking place. If and when the coils 46B and 48B become misaligned, then less than a maximum energy transfer occurs, and the voltage V_{S} sensed by detection circuit 704 increases significantly. If the voltage V_{S} reaches a predetermined level, alignment indicator 706 is activated via an audible speaker and/or LEDs for visual feedback. After adjustment, when an optimum energy transfer condition is established, causing Vs to decrease below the predetermined threshold level, the alignment indicator 706 is turned off.

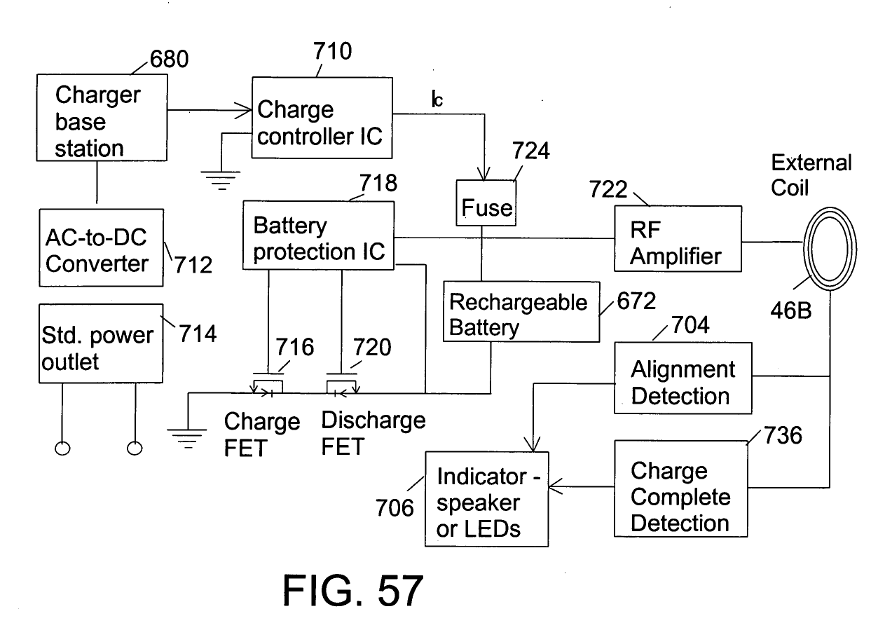

The elements of the external recharger are shown as a block diagram in conjunction with FIG. 57. In this disclosure, the words charger and recharger are used interchangeably. The charger base station 680 receives its energy from a standard power outlet 714, which is then converted to 5 volts DC by a AC-to-DC transformer 712. When the re-charger is placed in a charger base station 680, the re-chargeable battery 672 of the re-charger is fully recharged in a few hours and is able to recharge the battery 694 of the IPG 391R. If the battery 672 of the external re-charger falls below a prescribed limit of 2.5 volt DC, the battery 672 is trickle charged until the voltage is above the prescribed limit, and then at that point resumes a normal charging process.

As also shown in FIG. 57, a battery protection circuit 718 monitors the voltage condition, and disconnects the battery 672 through one of the FET switches 716, 720 if a fault occurs until a normal condition returns. A fuse 724 will disconnect the battery 672 should the charging or discharging current exceed a prescribed amount.

In summary, in the method of the current invention for neuromodulation of cranial nerve such as the occipital nerves to provide adjunct therapy for involuntary movement disorders (including Parkinson's disease and epilepsy) be practiced with any of the several pulse generator systems disclosed including,

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a programmable implantable pulse generator;

e) a combination implantable device comprising both a stimulus-receiver and a programmable IPG; and

f) an IPG comprising a rechargeable battery.

Neuromodulation of occipital nerves with any of these systems is considered within the scope of this invention.

In one embodiment, the external stimulator and/or the programmer has a telecommunications module, as described in a co-pending application, and summarized here for reader convenience. The telecommunications module has two-way communications capabilities.

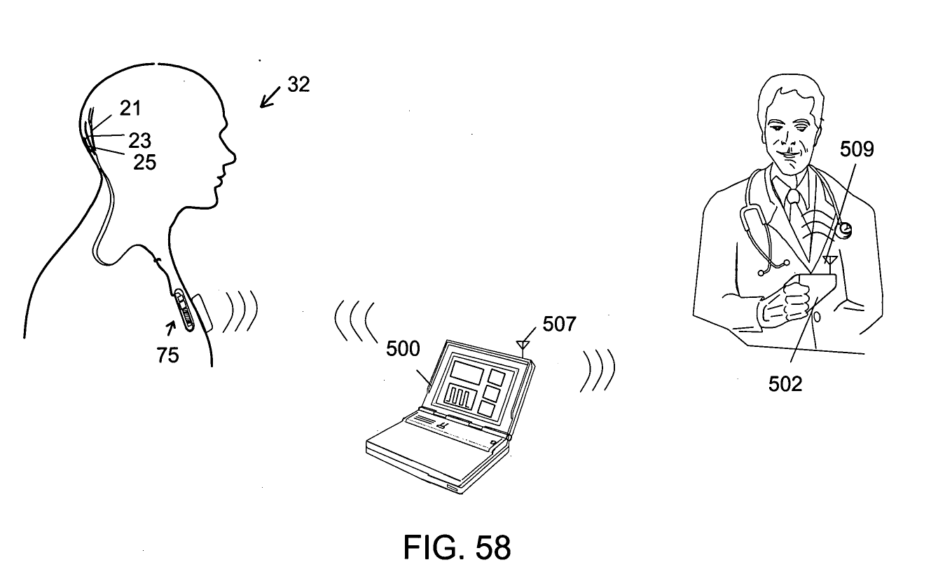

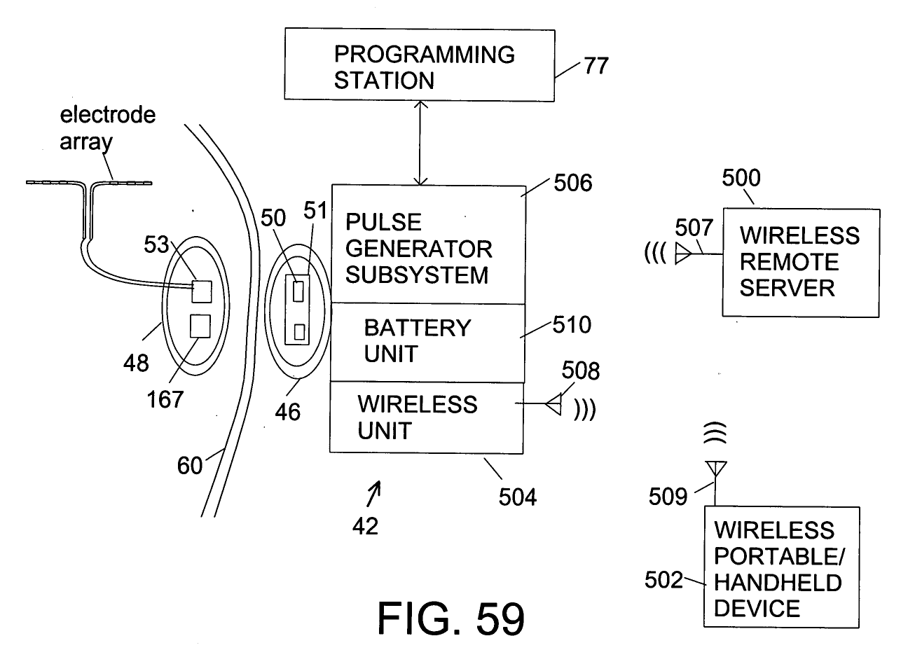

FIGS. 58 and 59 depict communication between an external stimulator 42 and a remote hand-held computer 502. A desktop or laptop computer can be a server 500 which is situated remotely, perhaps at a physician's office or a hospital. The stimulation parameter data can be viewed at this facility or reviewed remotely by medical personnel on a hand-held personal data assistant (PDA) 502, such as a “palm-pilot” from PALM corp. (Santa Clara, Calif.), a “Visor” from Handspring Corp. (Mountain view, Calif.) or on a personal computer (PC). The physician or appropriate medical personnel, is able to interrogate the external stimulator 42 device and know what the device is currently programmed to, as well as, get a graphical display of the pulse train. The wireless communication with the remote server 500 and hand-held PDA 502 would be supported in all geographical locations within and outside the United States (US) that provides cell phone voice and data communication service.

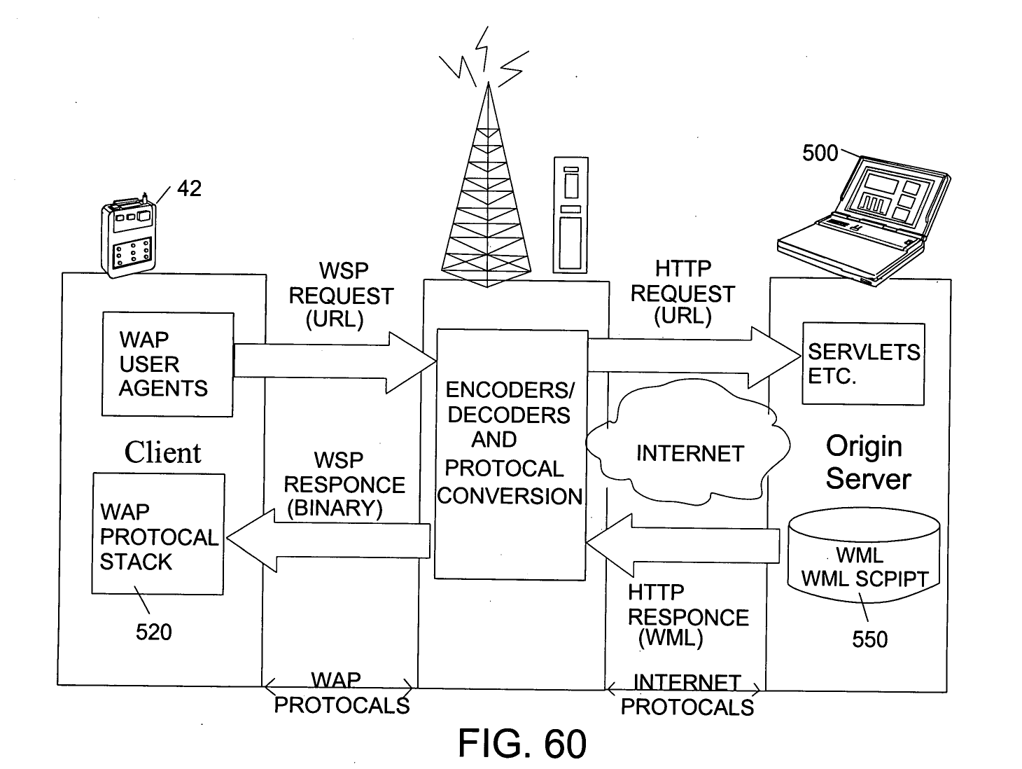

In one aspect of the invention, the telecommunications component can use Wireless Application Protocol (WAP). The Wireless Application Protocol (WAP), which is a set of communication protocols standardizing Internet access for wireless devices. While previously, manufacturers used different technologies to get Internet on hand-held devices, with WAP devices and services interoperate. WAP also promotes convergence of wireless data and the Internet. The WAP programming model is heavily based on the existing Internet programming model, and is shown schematically in FIG. 60. Introducing a gateway function provides a mechanism for optimizing and extending this model to match the characteristics of the wireless environment. Over-the-air traffic is minimized by binary encoding/decoding of Web pages and readapting the Internet Protocol stack to accommodate the unique characteristics of a wireless medium such as call drops.

The key components of the WAP technology, as shown in FIG. 60, includes 1) Wireless Mark-up Language (WML) 550 which incorporates the concept of cards and decks, where a card is a single unit of interaction with the user. A service constitutes a number of cards collected in a deck. A card can be displayed on a small screen. WML supported Web pages reside on traditional Web servers. 2) WML Script which is a scripting language, enables application modules or applets to be dynamically transmitted to the client device and allows the user interaction with these applets. 3) Microbrowser, which is a lightweight application resident on the wireless terminal that controls the user interface and interprets the WMLNVMLScript content. 4) A lightweight protocol stack 520 which minimizes bandwidth requirements, guaranteeing that a broad range of wireless networks can run WAP applications. The protocol stack of WAP can comprise a set of protocols for the transport (WTP), session (WSP), and security (WTLS) layers. WSP is binary encoded and able to support header caching, thereby economizing on bandwidth requirements. WSP also compensates for high latency by allowing requests and responses to be handled asynchronously, sending before receiving the response to an earlier request. For lost data segments, perhaps due to fading or lack of coverage, WTP only retransmits lost segments using selective retransmission, thereby compensating for a less stable connection in wireless. The above mentioned features are industry standards adopted for wireless applications and greater details have been publicized, and well known to those skilled in the art.

In this embodiment, two modes of communication are possible. In the first, the server initiates an upload of the actual parameters being applied to the patient, receives these from the stimulator, and stores these in its memory, accessible to the authorized user as a dedicated content driven web page. The physician or authorized user can make alterations to the actual parameters, as available on the server, and then initiate a communication session with the stimulator device to download these parameters.

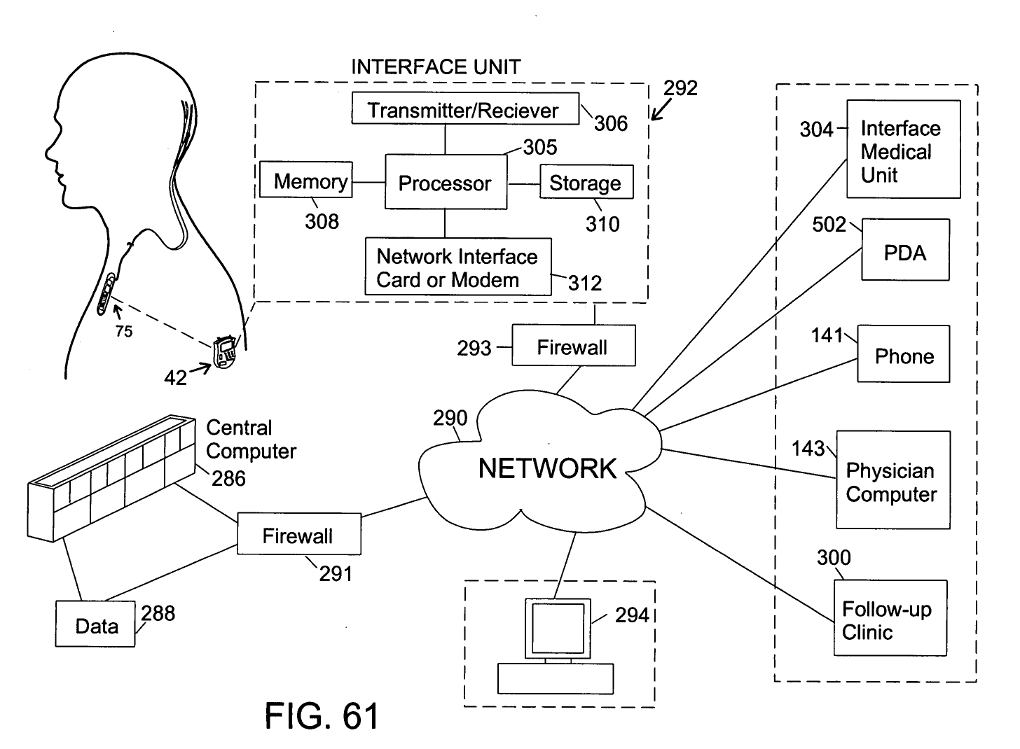

Shown in conjunction with FIG. 61, in one embodiment, the external stimulator 42 and/or the programmer 85 may also be networked to a central collaboration computer 286 as well as other devices such as a remote computer 294, PDA 502, phone 141, physician computer 143. The interface unit 292 in this embodiment communicates with the central collaborative network 290 via land-lines such as cable modem or wirelessly via the internet. A central computer 286 which has sufficient computing power and storage capability to collect and process large amounts of data, contains information regarding device history and serial number, and is in communication with the network 290. Communication over collaboration network 290 may be effected by way of a TCP/IP connection, particularly one using the internet, as well as a PSTN, DSL, cable modem, LAN, WAN or a direct dial-up connection.

The standard components of interface unit shown in block 292 are processor 305, storage 310, memory 308, transmitter/receiver 306, and a communication device such as network interface card or modem 312. In the preferred embodiment these components are embedded in the external stimulator 42 and can also be embedded in the programmer 85. These can be connected to the network 290 through appropriate security measures (Firewall) 293.

Another type of remote unit that may be accessed via central collaborative network 290 is remote computer 294. This remote computer 294 may be used by an appropriate attending physician to instruct or interact with interface unit 292, for example, instructing interface unit 292 to send instruction downloaded from central computer 286 to remote implanted unit.

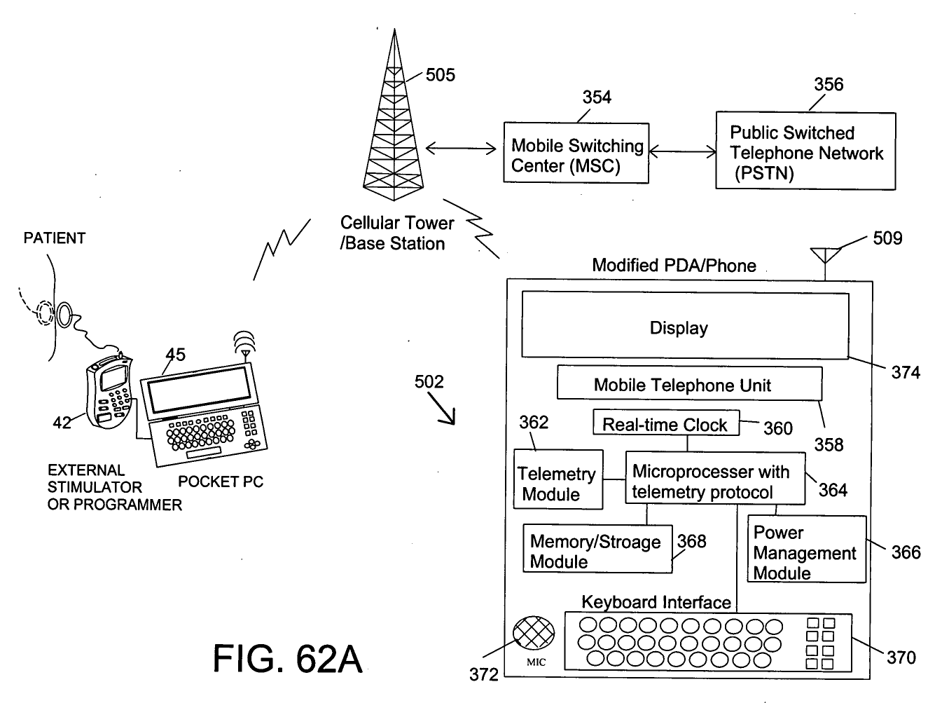

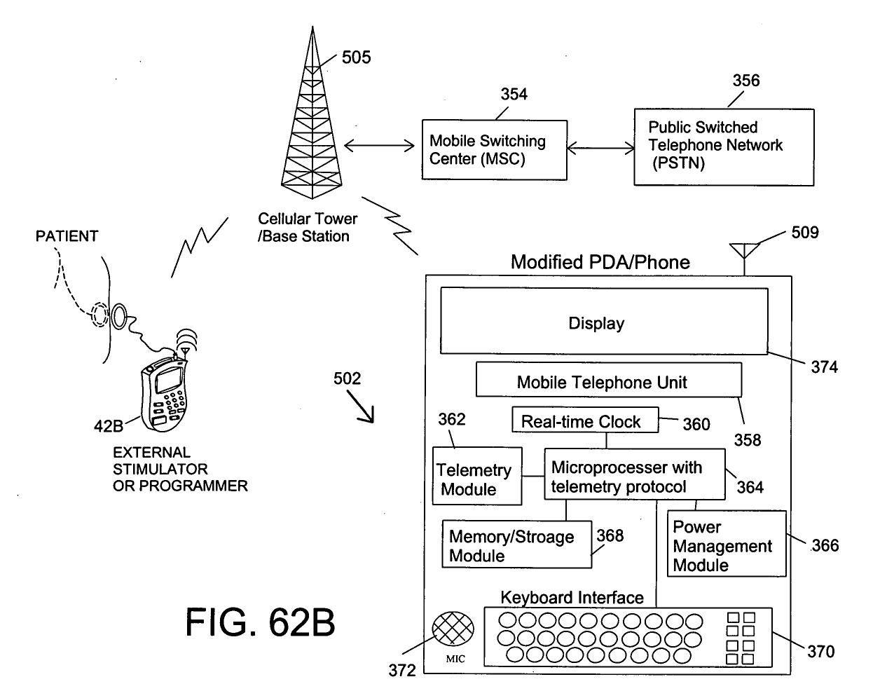

Shown in conjunction with FIGS. 62A and 62B the physician's remote communication's module is a Modified PDA/Phone 502 in this embodiment. The Modified PDA/Phone 502 is a microprocessor based device as shown in a simplified block diagram in FIGS. 62A and 62B. The PDA/Phone 502 is configured to accept PCM/CIA cards specially configured to fulfill the role of communication module 292 of the present invention. The Modified PDA/Phone 502 may operate under any of the useful software including Microsoft Window's based, Linux, Palm OS, Java OS, SYMBIAN, or the like.

The telemetry module 362 comprises an RF telemetry antenna 142 coupled to a telemetry transceiver and antenna driver circuit board which includes a telemetry transmitter and telemetry receiver. The telemetry transmitter and receiver are coupled to control circuitry and registers, operated under the control of microprocessor 364. Similarly, within stimulator a telemetry antenna 142 is coupled to a telemetry transceiver comprising RF telemetry transmitter and receiver circuit. This circuit is coupled to control circuitry and registers operated under the control of microcomputer circuit.

With reference to the telecommunications aspects of the invention, the communication and data exchange between Modified PDA/Phone 502 and external stimulator 42 operates on commercially available frequency bands. The 2.4-to-2.4853 GHz bands or 5.15 and 5.825 GHz, are the two unlicensed areas of the spectrum, and set aside for industrial, scientific, and medical (ISM) uses. Most of the technology today including this invention, use either the 2.4 or 5 GHz radio bands and spread-spectrum technology.

The telecommunications technology, especially the wireless internet technology, which this invention utilizes in one embodiment, is constantly improving and evolving at a rapid pace, due to advances in RF and chip technology as well as software development. Therefore, one of the intents of this invention is to utilize “state of the art” technology available for data communication between Modified PDA/Phone 502 and external stimulator 42. The intent of this invention is to use 3G technology for wireless communication and data exchange, even though in some cases 2.5G is being used currently.

For the system of the current invention, the use of any of the “3G” technologies for communication for the Modified PDA/Phone 502, is considered within the scope of the invention. Further, it will be evident to one of ordinary skill in the art that as future 4 G systems, which will include new technologies such as improved modulation and smart antennas, can be easily incorporated into the system and method of current invention, and are also considered within the scope of the invention.

The present invention may be embodied in other specific forms without departing from the spirit or essential attributes thereof. It is therefore desired that the present embodiment be considered in all aspects as illustrative and not restrictive, reference being made to the appended claims rather than to the foregoing description to indicate the scope of the invention.

We claim:

1. A method of providing rectangular and/or complex electrical pulses to at least one of greater occipital nerve, lesser occipital nerve, third occipital nerve, and tissues surrounding said nerves, to provide therapy or alleviate the symptoms for at least one of chronic headaches, transformed migraines, and occipital neuralgias, comprising the steps of:
providing pulse generation means for generating and emitting rectangular and complex electrical pulses, wherein said complex electrical pulses comprise at least one of multi-level pulses, biphasic pulses, non-rectangular pulses, or pulses with varying amplitude during the pulse;
providing at least one lead in electrical contact with said pulse generation means; and
providing at least one electrode at the distal end of said lead, wherein said at least one electrode is adapted to be in electrical contact with at least one of said greater occipital nerve, lesser occipital nerve, third occipital, and tissues surrounding said nerves.

2. The method of claim 1, wherein said pulse generation means for providing said rectangular and complex electric pulses is one from a group comprising: i) an external pulse generator coupled to an implanted passive stimulus-receiver; ii) an external stimulator used in conjunction with an implanted stimulus-receiver comprising a high value capacitor for storing electric charge; iii) a programmer-less implantable pulse generator (IPG) which is operable with a magnet; iv) a programmable implantable pulse generator (IPG); v) a combination implantable device comprising both a programmable implantable pulse generator (IPG) and a stimulus-receiver; vi) a programmable implantable pulse generator (IPG) comprising a rechargeable battery.

3. The method of claim 1, wherein said pulse generation means further comprises at least two predetermined/pre-packaged programs stored in memory, wherein said predetermined/pre-packaged programs define the variable parameters comprising, pulse amplitude, pulse width, pulse frequency, electrode pair selection, on-time and off-time sequence.

4. The method of claim 3, wherein said at least two predetermined/pre-packaged programs stored in said memory can be modified.

5. The method of claim 1, wherein said pulse generation means further comprises telemetry means for remote interrogation and/or remote programming over a wide area network.

6. The method of claim 1, wherein rectangular and/or complex electrical pulses may be provided to block said at least one of greater occipital nerve, lesser occipital nerve, third occipital nerve and tissue around said nerves with at least one of DC anodal block, Wedenski block, and Collision block.

7. The method of claim 1, wherein said at least one electrode is from a group comprising of button electrodes, cylindrical electrodes, and drug-eluting electrodes.

8. The method of claim 1, wherein said pulses further comprise pulse amplitude between 0.1 volt-15 volts; pulse width between 20 micro-seconds-5 milli-seconds; stimulation frequency between 5 Hz and 200 Hz, and blocking frequency between 0 and 750 Hz.

9. A method of providing therapy for at least one of chronic headaches, transformed migraines, and occipital neuralgias by stimulating and/or blocking at least one of greater occipital nerve, lesser occipital nerve, third occipital nerve, and tissues surrounding said nerves with predetermined/pre-packaged programs, comprising the steps of:
providing a pulse generation means capable of emitting electrical pulses;
providing at least two said predetermined/pre-packaged programs of therapy, wherein said predetermined/pre-packaged programs define the variable parameters comprising, pulse amplitude, pulse width, pulse frequency, electrode pair selection, on-time and off-time sequence;
providing at least one lead in electrical contact with said pulse generation means;
providing at least one electrode at the distal end of said lead wherein said at least one electrode is adapted to be in electrical contact with at least one of greater occipital nerve, lesser occipital nerve, third occipital nerve, and tissues surrounding said nerves; and
activating one of said at least two predetermined/pre-packaged programs, whereby said stimulation and/or blocking is provided according to said at least two predetermined/pre-packaged programs.

10. The method of claim 9, wherein said wherein said pulse generation means for providing said rectangular and complex electric pulses is one from a group comprising: i) an external pulse generator coupled to an implanted passive stimulus-receiver; ii) an external stimulator used in conjunction with an implanted stimulus-receiver comprising a high value capacitor for storing electric charge; iii) a programmer-less implantable pulse generator (IPG) which is operable with a magnet; iv) a programmable implantable pulse generator (IPG); v) a combination implantable device comprising both a programmable implantable pulse generator (IPG) and a stimulus-receiver; vi) a programmable implantable pulse generator (IPG) comprising a rechargeable battery.

11. The method of claim 9, wherein said pulses further comprise pulse amplitude between 0.1 volt-15 volts; pulse width between 20 micro-seconds-5 milli-seconds; stimulation frequency between 5 Hz and 200 Hz, and blocking frequency between 0 and 750 Hz.

12. The method of claim 9, wherein said at least two predetermined/pre-packaged programs stored in said memory can be modified.

13. The method of claim 9, wherein said pulse generation means further comprises telemetry means for remote interrogation and/or remote programming over a wide area network.

14. A method of providing therapy for at least one of chronic headaches, transformed migraines, and occipital neuralgias, comprising the steps of:
providing a pulse generation means capable of emitting rectangular and complex electrical pulses, wherein complex electrical pulses comprises one of non-rectangular pulses, multi-level pulses, biphasic pulses, or pulses with varying amplitude during the pulse;
selecting said pulse generation means from a group comprising: i) an external pulse generator coupled to an implanted passive stimulus-receiver; ii) an external stimulator used in conjunction with an implanted stimulus-receiver comprising a high value capacitor for storing electric charge; iii) a programmer-less implantable pulse generator (IPG) which is operable with a magnet; iv) a programmable implantable pulse generator (IPG); v) a combination device comprising both a programmable implantable pulse generator (IPG) and a stimulus-receiver; vi) a programmable implantable pulse generator (IPG) comprising a rechargeable battery.
providing an implanted lead adapted to be in electrical connection with said pulse generation; and
providing at least one electrode at the distal end of said lead, wherein said at least one electrode is adapted to be in contact with at least one of greater occipital nerve, lesser occipital nerve, third occipital nerve, and tissues surrounding said nerves to deliver said electrical pulses.

15. The method of claim 14, wherein said pulse generation means further comprises at least two predetermined/pre-packaged programs stored in memory, wherein said predetermined/pre-packaged programs define the variable parameters comprising, pulse amplitude, pulse width, pulse frequency, electrode pair selection, on-time and off-time sequence.

16. The method of claim 15, wherein said at least two predetermined/pre-packaged programs stored in said memory can be modified.

17. The method of claim 14, wherein said pulse generation means further comprises telemetry means for remote interrogation and/or remote programming over a wide area network.

18. The method of claim 14, wherein said at least one electrode is from a group comprising button electrodes, cylindrical electrodes, and drug-eluting electrodes.

19. The method of claim 14, wherein said pulses further comprise pulse amplitude between 0.1 volt-15 volts; pulse width between 20 micro-seconds-5 milli-seconds; stimulation frequency between 5 Hz and 200 Hz, and blocking frequency between 0 and 750 Hz.
